(19) United States
(12) Patent Application Publication
(10) Pub. No.: US 2014/0349396 A1
(43) Pub. Date: Nov. 27, 2014

(54) Compositions and Methods Relating to Clonal Progenitor Cells

(71) Applicants: Michael West, Mill Valley, CA (US); James T Murai, San Bruno, CA (US); Jay Yang, Palo Alto, CA (US)

(72) Inventors: Michael West, Mill Valley, CA (US); James T Murai, San Bruno, CA (US); Jay Yang, Palo Alto, CA (US)

(21) Appl. No.: 14/238,160

(22) PCT Filed: Sep. 10, 2012
(86) PCT No.: PCT/US12/54525

§ 371 Date: Apr. 29, 2014

Related U.S. Application Data
(60) Provisional application No. 61/533,127, filed on Sep. 9, 2011.
(60) Provisional application No. 61/609,162, filed on Mar. 9, 2012.

(51) Int. Cl. C12N 5/0735; C12N 5/071

(52) U.S. Cl. CPC C12N 5/0606; C12N 5/069
USPC 435/366; 435/325; 435/377

(57) ABSTRACT

Aspects of the present invention include methods and compositions related to the production and use of clonal lineages of embryonic progenitor cell lines derived from differentiating cultures of primordial stem cells. In particular, said methods and compositions relate to methods of differentiating cells in the presence of agents that inhibit the signaling of the TGF beta family members of growth factors and the applications of said cell lines in the treatment of diseases such as degenerative muscle disorders, cancer, and vascular disease.

This application claims priority to U.S. Provisional Application No. 61/533,127 filed on Sep. 9, 2011 and U.S. Provisional Application No. 61/609,162 filed on Mar. 9, 2012 both of which are incorporated by reference in their entirety.

### BACKGROUND

Advances in stem cell technology, such as the isolation and propagation in vitro of primordial stein cells, including embryonic stem cells (“ES” cells including human ES cells (“hES” cells)) and related primordial stem cells including but not limited to, IPS, EG, EC, ICM, epiblast, or ED cells (including human iPS, EG, EC, ICM, epiblast, or ED cells), constitute an important new area of medical research. hES cells have a demonstrated potential to be propagated in the undifferentiated state and then to be induced subsequently to differentiate into likely any and all of the cell types in the human body, including complex tissues. In addition, many of these primordial stem cells are naturally telomerase positive in the undifferentiated state, thereby allowing the cells to be expanded indefinitely. This expansion potential allows these primordial cells to be genetically modified followed by clonal expansion of the successfully modified cells, thus permitting the large-scale expansion of homogeneous populations of genetically modified primordial stem cells from which genetically-modified cells of all human somatic cell lineages can be made. Since the telomere length of many of these cells is comparable to that observed in sperm DNA (approximately 10-18 kb TRF length), differentiated cells derived from these immortal lines once they begin differentiation (generally associated with the repression of the expression of the catalytic component of telomerase (TERI)) display a long initial telomere length providing the cells with a long replicative capacity compared to cells isolated from fetal or adult-derived tissue. This has led to the suggestion that many diseases resulting from the dysfunction of cells may be amenable to treatment by the administration of hES-derived cells of various differentiated types (Thomson et al., Science 282:1145-1147 (1998)) including those with or without genetic modifications.

Nuclear transfer studies have demonstrated that it is possible to transform a somatic differentiated cell back to a primordial stem cell state such as that of embryonic stem (“ES”) cells (Cibelli et al., Nature Biotech 16:642-646 (1998)) or embryo-derived (“ED”) cells. The development of technologies to reprogram somatic cells back to a totipotent ES cell state herein collectively designated “iPS cell technologies”, such as by the transfer of the genome of the somatic cell to an enucleated oocyte and the subsequent culture of the reconstructed embryo to yield ES cells, often referred to as somatic cell nuclear transfer (“SCNT”) or through analytical reprogramming technology, offers methods to transplant ES-derived somatic cells with a nuclear genotype of the patient (Lanza et al., Nature Medicine 5:975-977 (1999)).

In addition to SCNT, other techniques exist to address the problem of transplant rejection, including the use of gynogenesis and androgenesis (see U.S. application No. 60/161,987, filed Oct. 28, 1999; Ser. No. 09/697,297, filed Oct. 27, 2000; Ser. No. 09/995,659, filed Nov. 29, 2001; Ser. No. 10/374,512, filed Feb. 27, 2003; PCT application no. PCT/US00/29551, filed Oct. 27, 2000; the disclosures of which are incorporated by reference in their entirety). In the case of a type of gynogenesis designated parthenogenesis, pluripotent stem cells may be manufactured without antigens foreign to the gamete donor and therefore useful in manufacturing cells that can be transplanted without rejection. In addition, parthenogenic stem cell lines can be assembled into a bank of cell lines homozygous in the HLA region (or corresponding MHC region of nonhuman animals) to reduce the complexity of a stem cell bank in regard to HLA haplotypes.

In addition, pluripotent stem cell lines or a bank of said cell lines can be produced that are hemizygous in the HLA region (or corresponding WIC region of nonhuman animals; see PCT application Ser. No. PCT/US2006/040985 filed Oct. 20, 2006 entitled “Totipotent, Nearly Totipotent or Pluripotent Mammalian Cells Homozygous or Hemizygous for One or More Histocompatibility Antigen Genes”, incorporated herein by reference). A bank of hemizygous cell lines provides the advantage of not only reducing the complexity inherent in the normal mammalian MHC gene pool, but it also reduces the gene dosage of the antigens to reduce the expression of said antigens without eliminating their expression entirely, thereby not stimulating a natural killer response.

In addition to SCNT, parthenogenesis, and the construction of banks of cells with homozygous or hemizygous HLA alleles, other techniques exist to address the problem of transplant rejection, including the use of technologies to reprogram somatic cells using transcriptional regulators (see PCT application Ser. No. PCT/US2006/030632 filed on Aug. 3, 2006 and titled “Improved Methods of Reprogramming Animal Somatic Cells”, incorporated herein by reference) including cells reprogrammed while downregulating SP100 expression (see U.S. Provisional Patent Application 61/492,329 filed on Jun. 6, 2011 and titled “Embryonic Stem Cell and Embryonic Progenitor-Associated Molecules Useful in the Management of Cancer and Cellular Reprogramming”, incorporated herein by reference in its entirety). In addition, methods of genetically modifying cells to induce immune tolerance are known in the art including the introduction of nonclassical class I molecules such as human leucocyte antigen (HLA) E or G can be expressed that block natural killer cell action and induce tolerance (Carosella et al., 2001 HLA-G: a shield against inflammatory, aggression. Trends Immunol. 22, 553-555; Wiendl et al., 2003 The nonclassical MHC molecule HLA-G protects human muscle cells from immune-mediated lysis: implications for myoblast transplantation and gene therapy. Brain 126, 176-185; PCT application Ser. No. PCT/US2008/058779 filed on Mar. 28, 2008 and titled “ENDOGENOUS EXPRESSION OF HLA-G AND/OR HLA-E BY MESENCHYMAL CELLS”; PCT Application Ser. No. PCT/US2009/047647 filed Jun. 17, 2009 and titled “DIFFERENTIATION OF MESENCHYMAL STEM CELLS INTO FIBROBLASTS, COMPOSITIONS COMPRISING MESENCHYMAL STEM CELL-DERIVED FIBROBLASTS, AND METHODS OF USING THE SAME”

The potential to clonally isolate lines of human embryonic progenitor (hEP) cell lines provides a means to propagate diverse and novel highly purified cell lineages useful in the production of diverse secreted factors, for research, and cells for use in the manufacture of cell-based therapies (see PCT application Ser. No. PCT/US2006/013519 filed on Apr. 11, 2006 and titled “Novel Uses of Cells With Prenatal Patterns of Gene Expression”; U.S. patent application Ser. No. 11/604,047 filed on Nov. 21, 2006 and titled “Methods to Accelerate the Isolation of Novel Cell Strains from Pluripotent Stem Cells and Cells Obtained Thereby”; U.S. patent application Ser. No. 12/504,630 filed on Jul. 16, 2009 and titled “Methods to Accelerate the Isolation of Novel Cell Strains from Pluripotent Stein Cells and Cells Obtained Thereby”; and U.S. patent application Ser. No. 13/115,688 filed on May 25, 2011 and titled “Improved Methods of Screening Embryonic Progenitor Cell Lines”, each of which is incorporated herein by reference).

Nevertheless, there remains a need for novel methods to isolate new monoclonal, oligoclonal, or pooled oligoclonal hEP cell lines, including vascular endothelial cells and skeletal muscle myoblasts, and means to differentiate monoclonal, oligoclonal, or pooled oligoclonal hEP cell lines under conditions which are compatible in either a general laboratory setting or in a good manufacturing processes (“GMP”) cell manufacturing facility where there is adequate documentation as to the purity and genetic normality of the cells at advanced passages (>18-21 doublings of clonal expansion). In particular, there remains a need for improved methods of isolating, propagating, or differentiating said hEP cell lines that are difficult to expand in the presence of TGF beta growth factors.

### SUMMARY OF THE INVENTION

We have previously demonstrated that the long initial telomere length of hES cells, together with the unexpected robust proliferative capacity of primitive hES-derived progenitor cell types, facilitates the industrial expansion and characterization of >140 diverse and scalable clonal lineages with diverse defined homeobox gene expression as well as diverse transcriptional regulators (West et al., 2008, Reg Med 3(3) pp. 287-308), incorporated herein by reference, including supplemental information; and U.S. patent application Ser. No. 12/504,630 filed on Jul. 16, 2009 and titled “Methods to Accelerate the Isolation of Novel Cell Strains from Pluripotent Stein Cells and Cells Obtained Thereby”, incorporated herein by reference in its entirety). The robustness of these clonally-purified lines, their ability to expand for >40 passages while maintaining their pattern of gene expression, lack of tumorigenicity, and their embryonic pattern of gene expression offers novel compositions and methods for modeling numerous differentiation pathways for the first time in vitro, and for the manufacture of purified product not existing in such a purified state in nature or using other manufacturing modalities. We disclose herein novel compositions and methods produced using these methods while blocking TGF beta signaling, to produce novel monoclonal embryonic progenitor cell lines including diverse types of skeletal muscle myoblasts and vascular endothelial cells.

In one embodiment, pluripotent stem cells such as hES cells or iPS cells are cultured in conditions previously disclosed to increase the frequency of obtaining VE-cadherin positive vascular endothelial cells (see U.S. Provisional Application No. 61/290,667, filed on Dec. 29, 2009 and titled “Methods For Developing Endothelial Cells From Pluripotent Cells And Endothelial Cells Derived”, incorporated herein by reference) with the additional step that said cells are cloned as monoclonal cell lineages. More specifically, hES cells are cultured as EBs in the presence of an activin, and a BMP and FGF-2 for a time sufficient for mesoderm induction. In a specific embodiment, the activin is activin A, the BMP is BMP4, and EBs are cultured for 4-6 days with the growth factors added at appropriate time to the culture media. In another embodiment, following mesoderm induction, the cells are plated on an adherent substrate and cultured in media such as MCDB 131 supplemented with 5.0 ng/mL VEGF-A, 5.0 ng/mL, FGF-2, 0.75 IU/mL, heparin, 2% FBS (such as Promocell endothelial MV2 media with supplements at concentrations normally recommended by the manufacturer and sold as a complete kit (Cat# C-22022) or as cell basal medium (Cat# C-22221) and growth supplement (Cat# C-39221)) and a TGFβ signaling inhibitor to induce and propagate vascular endothelium, or basal media such as MCDB 120 supplemented with 10 ng/mL EGF, 10 ug/mL insulin, 1 ng/mL FGF-2, 0.4 ug/mL, dexamethasone, 50 ug/mL fetuin, 5% FBS (such as Promocell skeletal muscle basal medium (Cat# C-22060B) and growth supplement (Cat# C-39365)) and a TGFβ signaling inhibitor to induce and propagate skeletal muscle myoblasts. In a specific embodiment, the cells are differentiated as outlined in FIG. 1 for 3-7 days. In some embodiments, following induction of vascular differentiation, the cells are cultured hi media supplemented with VEGF-A, FGF-2 and a TGFβ signaling inhibitor for at least 4-5 days, preferably at least 5-7 days, to sufficiently expand the vascular endothelial or skeletal muscle myoblasts in the cell population.

In another embodiment, pluripotent stem cells, such as hES or iPS cells, are differentiated in vitro in the presence of Activin-A and WNT-3A followed by FGF-4 and BMP-2 and then cloned as monoclonal cell lineages on Matrigel, gelatin, or similar supportive culture support in the presence of media capable of supporting the growth of vascular endothelial cells. In some instances, hES or iPS cells are cultured as colonies on fibroblast feeder cells that are allowed to overgrow and differentiate in situ for 13 days in ES cell culture medium such as Invitrogen KO-DMEM with KO-serum replacement. Then, on differentiation day 0 (FIG. 6), media is changed to a basal differentiation media comprising KO-DMEM/RPMI-1640 (5/1 v/v) and said basal differentiation media is supplemented with 100 ng/mL Activin A and 25 ng/mL Wnt3A. On the beginning of day 2 (designated Day 1 in FIG. 6), and for the following two days the media is replaced with the said basal differentiation medium supplemented only with 100 ng/mL Activin A. Then on the beginning of day 4 (designated Day 3 on FIG. 6), the media is replaced with the said basal differentiation media supplemented with 30 ng/mL FGF4 and 20 ng/mL BMP2. At the beginning of Day 8 (designated Day 7 in FIG. 6), cells are rinsed twice in PBS and disaggregated with Accutase, and plated on Matrigel-coated plates in medium capable of supporting the proliferation of vascular endothelial cells supplemented with a TGFβ signaling inhibitor such as SB431542. A non-limiting example of said endothelial media MCDB 131 supplemented with 5.0 ng/mL VEGF-A, 5.0 ng/mL FGF-2, 0.75 IU/mL heparin, 2% FBS (such as Promocell endothelial MV2 media with supplements at concentrations normally recommended by the manufacturer and sold as a complete kit (Cat# C-22022) or as cell basal medium (Cat# C-22221) and growth supplement (Cat# C-39221)) and a TGF signaling inhibitor such as SB431542. Cells are expanded as working stocks of candidate cultures that can be expanded and cryopreserved for the purposes of deriving continuous clonal cell lines. The candidate cultures are plated at approximately 500 and 2,000 cells in 15 cm tissue culture dishes coated with Matrigel or suitable substrate for the culture of endothelial cells, and allowed to grow to visible cell colonies which are subsequently isolated by various means known in the art such as the use of cloning cylinders, and serially propagated as cell lines which are then expanded in the same media and matrix, and cryopreserved for future use. Uses of said cells, in particular, those that have been produced in a manner such that the cells may be permanently engrafted in the host without rejection, including but not limited to those produced from iPS cells, that express vascular endothelial markers such as PECAM1, CDH5 (VE-Cadherin), and vWF include transplantation to increase blood flow in ischemic or aged tissues such as ischemic myocardium or ischemic limbs. Particularly useful are clonal, pooled clonal, oligoclonal, or pooled oligoclonal endothelial cell lines that express relatively high levels of ITLN1 (Omentin) or ITLN2 and are useful in imparting increased sensitivity to insulin in Type II diabetes, aged, or Syndrome X patients. Said ITLN1-expressing endothelial cell lines may be injected in ischemic muscle such as cardiac or skeletal muscle or other sites in the body to both supply young replication-competent cells capable of regenerating neoangiogenesis, but also to secrete the protein products of the ITLN1 or ITLN2 gene or both genes to further promote vascularization, reduce inflammatory pathways, increase insulin sensitivity in said patients. The dosage of said cells will vary from patient to patient but can easily be determined by measuring the serum or plasma levels of Omentin in the patient. As has been reported (Zhong et al, Acta Pharmacol Sin 32: 873-878) serum omentin levels approximate 254 ng/ml+/−72.9 ng/ml in normal patients and are observed to be 113 ng/ml in patients with acute coronary syndrome, and 155 ng/ml in patients with stable angina pectoris. Plasma levels in normal patients have also been reported to be 370 ng/mL (de Souza Batista et al, Diabetes 56: 1655-1661), differences that may be attributable to differences in assay technique. Dosages will vary based on the site of injection and disease status of the patient, and in some instances may range from 1×10⁶ to 1×10⁹ cells/patient, formulated in a suitable buffer or matrix such as hydrogels composed of crosslinked hyaluronic acid and gelatin such as HyStem-Rx (BioTime, Alameda, Calif.). In another embodiment, clonal, pooled clonal, oligoclonal, or pooled oligoclonal endothelial cell lines that express relatively high levels of ITLN1 (Omentin) or ITLN2 and are useful in treating vascular calcification and/or osteoporosis (see e.g., Xie et al., “Omentin-1 attenuates arterial calcification and bone loss in osteoprotegerin-deficient mice by inhibition of RANKL expression,” Cardiovasc. Res. (2011) 92 (2): 296-306. Said ITLN1-expressing endothelial cell lines may be injected in ischemic muscle such as cardiac or skeletal muscle or other sites in the body to both supply young replication-competent cells capable of regenerating neoangiogenesis, but also to secrete the protein products of the ITLN1 or ITLN2 gene or both genes to achieve the desired therapeutic effect in said patients. The dosage of said cells will vary from patient to patient but can easily be determined by measuring the serum or plasma levels of Omentin in the patient, Dosages will vary based on the site of injection and disease status of the patient, and in some instances may range from 1×10⁶ to 1×10⁹ cells/patient, formulated in a suitable buffer or matrix such as hydrogels composed of crosslinked hyaluronic acid and gelatin such as HyStem-Rx (BioTime, Alameda, Calif.).

In specific embodiments, the TGFβ signaling inhibitor is an inhibitor specific for the type I TGFβ receptors. In some embodiments, the inhibitor is an inhibitor of ALK4, ALK5, and ALK7. In other embodiments, the inhibitor is an inhibitor of at least ALK5.

In one embodiment, the TGFβ signaling inhibitor is a soluble form of a type I receptor, an antibody directed to a type I receptor, or a small molecule compound. In specific embodiments, the inhibitor is a small molecule selected from SB-431542, A 83-01, D 4476, LY 364947, SB 525334, SD 208, and SJN 2511.

In another aspect, this disclosure is directed to a substantially pure population of vascular endothelial cells. The endothelial cells are characterized by expression of surface markers, VE-cadherin, CD31, and Integrin AvB3, and can proliferate and pass for extended culture periods without losing the characteristics of vascular endothelial cells.

In yet other embodiments the invention provides 2 cell populations comprising a first population of clonal vascular endothelial progenitor cells and a second population comprising pluripotent stem cells such as hES cells or iPS cells, wherein the first population is the in vitro progeny of at least a portion of the second population. The clonal progenitors can replicate and be passaged in vitro through multiple passages

In a further aspect, the instant disclosure provides a composition containing ES or iPS derived, e.g., hESC-derived endothelial cells, for example, a pharmaceutical composition that also includes one or more pharmaceutically acceptable carriers and diluents. In some embodiments the invention provides a clonal progenitor cell line wherein the clonal progenitor cell line expresses one or more genes expressed by endothelial cells wherein the clonal progenitor cell line has substantially the same genome as a line of pluripotent stem cells such as a line of hES cells or a line of iPS cells.

In still another aspect, this disclosure provides a method for repairing injured tissue hi a human subject based on administering to the subject a composition containing the vascular endothelial cells disclosed herein to promote vascularization.

In another aspect, this disclosure is directed to a substantially pure population of skeletal muscle myoblast cells. The myoblast cells are characterized by expression of the gene expression markers MYH3, ACTA1, MYOG, and MYH7, and can proliferate and pass for extended culture periods without losing the characteristics of skeletal muscle progenitor cells. In another embodiment the invention provides a clonal myoblast progenitor cell line wherein the myoblast clonal progenitor cell line expresses one or more genes expressed by a skeletal muscle cell and wherein the myoblast clonal progenitor cell line has substantially the same genome as a line of pluripotent stem cells, such a line of hES cells or a line of iPS cells.

In yet other embodiments the invention provides 2 cell populations comprising a first population of clonal myoblast progenitor cells and a second population comprising pluripotent stem cells such as hES cells or iPS cells, wherein the first population is the in vitro progeny of at least a portion of the second population. The clonal progenitors can replicate and be passaged in vitro through multiple passages.

In a further aspect, the instant disclosure provides a composition containing hESC-derived skeletal muscle myoblast cells, for example, a pharmaceutical composition that also includes one or more pharmaceutically acceptable carriers and diluents.

In still another aspect, this disclosure provides a method for repairing injured tissue in a human subject based on administering to the subject a composition containing the skeletal muscle myoblast cells disclosed herein to promote skeletal or cardiac muscle repair and regeneration.

### BRIEF DESCRIPTION OF THE DRAWINGS

FIG. 1: Outline of a protocol for the generation of candidate cultures for the generation of clonal embryonic progenitors in the presence of SB431542.

FIG. 2: FACS analysis of CD31 (Pecam-1), CD34, and VE-cadherin, and E-cadherin antigens on endothelial cell lines of the present invention compared to cells derived in heterogeneous differentiation conditions.

FIG. 3: FACS analysis of VEGFR3 and Integrin AvB3 antigens on endothelial cell lines of the present invention compared to cells derived in heterogeneous differentiation conditions.

FIG. 4: A). The MYH3 positive clonal embryonic progenitor cell line RP1-SKEL-8 propagated in the undifferentiated state in the presence of SB431542. B). The MYH3 positive clonal embryonic progenitor cell line RP1-SKEL-8 (passage 9) cultured 6 days in muscle differentiation conditions. Scale bar is 100 microns. White arrow shows multinucleated cell.

FIG. 5: A heat map comparing the gene expression of heterogeneous cultures of hES-derived endothelial cells (ONC110413 ESI 035 day 11, ONC-ESI-017 day 15, ESI-051-day 21 OncoCyte) with the clonal endothelial cell line of the present invention and diverse types of cultures adult-derived normal endothelial cells. Genes with no or low expression are shown in yellow, genes with relatively high expression are shown in red. Genes listed on right are as follows (from top to bottom): PROX1, CD86, CD86, SPN, SPN, IL13, ITGB1, CD36, KLF1, CD160, SLAMF1, PODXL, PDPN, FLT1, BSG, FLT4, ACD, SELE, GATA1, ITGB1, ACE, COLEC12, CLEC4M, FLT4, CLEC4C, COLEC11, SPN, PLXDC1, CD34, HBG1, STAB2, CLEC4C, S1PR2, CD300LG, ACE, ZNF71, NFE2, AGGF, CD300LG, CD34, FLT4, CD34, PVRL2, FABP6, FABP6, THSD1, THSD1, ITGB7, FABP5, AGGF1, VCAM1, VCAM1, ITGA4, CCBP2, KLF4, ITGB2, ITGB2, S1PR5, BSG, NRP1, PVRL2, PVRL2, TNFRSF10B, TNFRSF10B, ICAM1, ITGB1, EPOR, TAL1, EGFL7, TNFRSF10B, TNFRSF10A, THSD1, SELP, ITGB1, ITGB1, CD151, CD151, ACTN1, TNFRSF1A, CD68, COLEC11, COLEC11, ANTXR1, ANTXR1, KLF4, F3, F3, THBD, CD36, CD36, IL1B, NRP1, COLEC12, MCAM, S1PR1, STAB1, EGFL7, PROCR, FABP5, FABP5, KDR, SELE, ENG, TEK, TEK, LMO2, EMCN, TNFRSF1B, PODXL, LYVE1, VCAM1, ANTXR1, PROM1, PDPN, PODXL, PECAM1, CD34, CD34, ESAM, CDH5, CD93, ICAM2, and VWF.

FIG. 6: Outline of a protocol for generation of monoclonal lineages of purified PECAM1+, CDH5+, vWF+ endothelial cell lines from GMP-capable hES cells including lines expressing ITLN1.

FIG. 7A: Graphical representation of the expression of CDH5 in various clonal cell lines, as described in the experimental section below.

FIG. 7B: Graphical representation of the expression of ITLN1 in various endothelial cell lines, as described in the experimental section below.

FIG. 7C: Graphical representation of the expression of ITLN2 in various endothelial cell lines, as described in the experimental section below.

FIG. 8: Graphical representation of the expression of APLNR in various endothelial cell lines, as described in the experimental section below.

FIG. 9: Graphical representation of the expression of NPTX2 in various endothelial cell lines, as described in the experimental section below.

FIG. 10: Graphical representation of the expression of DLK1 in various endothelial cell lines, as described in the experimental section below.

FIG. 11 is a photomicrograph showing myoblasts grown on Matrigel® in the presence of a TGFβ inhibitor (SB431542)(top) and myoblasts switched to gelatin without the TGFβ inhibitor.

### DETAILED DESCRIPTION OF THE INVENTION

### Abbreviations

AFP—Alpha fetoprotein
BMP—Bone Morphogenic Protein
BRL—Buffalo rat liver
BSA—Bovine serum albumin
CD—Cluster Designation
cGMP—Current Good Manufacturing Processes
CNS—Central Nervous System
DMEM—Dulbecco's modified Eagle's medium
DMSO—Dimethyl sulphoxide
DPBS—Dulbecco's Phosphate Buffered Saline
EBs—Embryoid bodies
EC—Embryonal carcinoma
EC Cells—Embryonal carcinoma cells; hEC cells are human embryonal carcinoma cells
ECAPCs—Embryonic cutaneous adipocyte progenitor cells
ECM—Extracellular Matrix
ED Cells—Embryo-derived cells; hED cells are human ED cells
EDTA—Ethylenediamine tetraacetic acid
EG Cells—Embryonic germ cells; hEG cells are human EG cells
EP Cells—Embryonic progenitor cells are cells derived from primordial stem cells that are more differentiated than primordial stem cells, in that they no longer display markers such as SSEA4, TRA1-60 or TRA-1-81 seropositivity in the case of the human species, but have not fully differentiated. Embryonic progenitor cells correspond to the embryonic stages as opposed to the postnatal stage of development.
ES Cells—Embryonic stem cells; hES cells are human ES cells
FACS—Fluorescence activated cell sorting
FBS—Fetal bovine serum
FCS—Fetal calf serum
FGF—Fibroblast Growth Factor
GFP—Green Fluorescent Protein
GMP—Good Manufacturing Practices
hED Cells—Human embryo-derived cells
hEG Cells—Human embryonic germ cells are stem cells derived from the primordial germ cells of fetal tissue.
hEP Cells—Human embryonic progenitor cells are embryonic progenitor cells from the human species.
hiPS Cells—Human induced pluripotent stem cells are cells with properties similar to hES cells obtained from somatic cells after exposure to hES-specific transcription factors such as SOX2, KLF4, OCT4, MYC or NANOG, LIN28, OCT4, and SOX2,
HSE—Human skin equivalents are mixtures of cells and biological or synthetic matrices manufactured for testing purposes or for therapeutic application in promoting wound repair.
HUVEC—human umbilical vein endothelial cell
ICM—Inner cell mass of the mammalian blastocyst-stage embryo.
iPS Cells—For the purposes of this specification, Induced pluripotent stem cells are cells with properties similar to hES cells obtained from somatic cells after exposure to ES-specific transcription factors such as SOX2, KLF4, OCT4, MYC, or NANOG, LIN28, OCT4, and SOX2 or somatic cells or genomes reprogrammed to a pluripotential state by other means including somatic cell nuclear transfer or analytical reprogramming.
LOH—Loss of Heterozygosity
MEM—Minimal essential medium
miRNA—Micro RNA
MSC—Mesenchymal Stem Cell
NT—Nuclear Transfer
PBS—Phosphate buffered saline
PEGDA—Polyethylene glycol diacrylate
PS fibroblasts—Pre-scarring fibroblasts are fibroblasts derived from the skin of early gestational skin or derived from ED cells that display a prenatal pattern of gene expression in that they promote the rapid healing of dermal wounds without scar formation.
RA—Retinoic acid
RFU—Relative Fluorescence Units
SCNT—Somatic Cell Nuclear Transfer
SFM—Serum-Free Medium
SPF—Specific Pathogen-Free
SV40 Simian Virus 40
Tag—Large T′-antigen
T-EDTA—Trypsin EDTA

### DEFINITIONS

The term “analytical reprogramming technology” refers to a variety of methods to reprogram the pattern of gene expression of a somatic cell to that of a more pluripotent state, such as that of an iPS, ES, ED, EC or EG cell, wherein the reprogramming occurs in multiple and discrete steps and does not rely simply on the transfer of a somatic cell into an oocyte and the activation of that oocyte. Such techniques include the use of cytoplasm such as EC cell-derived cytoplasm that is enriched in factors such as OCT4, LIN28, SOX2, NANOG, KLF4, and modifications that decrease the expression of SP100 (see U.S. application no. 60/332,510, filed Nov. 26, 2001; Ser. No. 10/304,020, filed Nov. 26, 2002; PCT application no. PCT/US02/37899, filed Nov. 26, 2003; U.S. application No. 60/705,625, filed Aug. 3, 2005; U.S. application No. 60/729,173, filed Aug. 20, 2005; U.S. application No. 60/818,813, filed Jul. 5, 2006, PCT/US06/30632, filed Aug. 3, 2006; and U.S. Provisional Patent Application 61/492,329 filed on Jun. 6, 2011 and titled “Embryonic Stem Cell and Embryonic Progenitor-Associated Molecules Useful in the Management of Cancer and Cellular Reprogramming”, the disclosure of each of which is incorporated by reference herein).

The term “blastomere/morula cells” refers to blastomere or morula cells in a mammalian embryo or blastomere or morula cells cultured in vitro with or without additional cells including differentiated derivatives of those cells.

The term “cell expressing gene X”, “gene X is expressed in a cell” (or cell population), or equivalents thereof, means that analysis of the cell using a specific assay platform provided a positive result. The converse is also true (i.e., by a cell not expressing gene X, or equivalents, is meant that analysis of the cell using a specific assay platform provided a negative result). Thus, any gene expression result described herein is tied to the specific probe or probes employed in the assay platform (or platforms) for the gene indicated.

The term “cell line” refers to a mortal or immortal population of cells that is capable of propagation and expansion in vitro.

The term “clonal” or alternatively “monoclonal” refers to a population of cells obtained the expansion of a single cell into a population of cells all derived from that original single cells and not containing other cells.

The term “colony in situ differentiation” refers to the differentiation of colonies of cells (e.g., hES, hEG, hiPS, hEC or hED) in situ without removing or disaggregating the colonies from the culture vessel in which the colonies were propagated as undifferentiated stem cell lines. Colony in situ differentiation does not utilize the intermediate step of forming embryoid bodies, though embryoid body formation or other aggregation techniques such as the use of spinner culture may nevertheless follow a period of colony in situ differentiation.

The term “differentiated cells” when used in reference to cells made by methods of this invention from pluripotent stem cells refer to cells having reduced potential to differentiate when compared to the parent pluripotent stem cells. The differentiated cells of this invention comprise cells that could differentiate further (i.e., they may not be terminally differentiated).

The term “direct differentiation” refers to process of differentiating: blastomere cells, morula cells, ICM cells, ED cells, or somatic cells reprogrammed to an undifferentiated state (such as in the process of making iPS cells but before such cells have been purified in an undifferentiated state) directly without the intermediate state of propagating isolated undifferentiated stein cells such as hES cells as undifferentiated cell lines. A nonlimiting example of direct differentiation would be the culture of an intact human blastocyst into culture and the derivation of ED cells without the generation of a human ES cell line as was described (Bongso et al, 1994. Human Reproduction 9:2110).

The term “embryonic stem cells” (ES cells) refers to cells derived from the inner cell mass of blastocysts, blastomeres, or morulae that have been serially passaged as cell lines while maintaining an undifferentiated state (e.g. expressing TERT, OCT4, and SSEA and TRA antigens specific for ES cells of the species). The ES cells may be derived from fertilization of an egg cell with sperm or DNA, nuclear transfer, parthenogenesis, or by means to generate hES cells with hemizygosity or homozygosity in the MHC region. While ES cells have historically been defined as cells capable of differentiating into all of the somatic cell types as well as germ line when transplanted into a preimplantation embryo, candidate ES cultures from many species, including human, have a more flattened appearance in culture and typically do not contribute to germ line differentiation, and are therefore called “ES-like cells.” It is commonly believed that human ES cells are in reality “ES-like”, however, in this application we will use the term ES cells to refer to both ES and ES-like cell lines.

The term “histotypic culture” refers to cultured cells that are aggregated to create a three-dimensional structure with tissue-like cell density such as occurs in the culture of some cells over a layer of agar or such as occurs when cells are cultured in three dimensions in a collagen gel, sponge, or other polymers such as are commonly used in tissue engineering.

The term “human embryo-derived” (“hED”) cells refers to blastomere-derived cells, morula-derived cells, blastocyst-derived cells including those of the inner cell mass, embryonic shield, or epiblast, or other totipotent or pluripotent stem cells of the early embryo, including primitive endoderm, ectoderm, mesoderm, and neural crest and their derivatives up to a state of differentiation correlating to the equivalent of the first eight weeks of normal human development, but excluding cells derived from hES cells that have been passaged as cell fines (see, e.g., U.S. Pat. Nos. 7,582,479; 7,217,569; 6,887,706; 6,602,711; 6,280,718; and U.S. Pat. No. 5,843,780 to Thomson, incorporated herein by reference). The hED cells may be derived from preimplantation embryos produced by fertilization of an egg cell with sperm or DNA, nuclear transfer, or chromatin transfer, an egg cell induced to form a parthenote through parthenogenesis, analytical reprogramming technology, or by means to generate hES cells with hemizygosity or homozygosity in the HLA region.

The term “human embryonic germ cells” (hEG cells) refer to pluripotent stem cells derived from the primordial germ cells of fetal tissue or maturing or mature germ cells such as oocytes and spermatogonia) cells, that can differentiate into various tissues in the body. The hEG cells may also be derived from pluripotent stem cells produced by gynogenetic or androgenetic means, i.e., methods wherein the pluripotent cells are derived from oocytes containing only DNA of male or female origin and therefore will comprise all female-derived or male-derived DNA (see U.S. application no. 60/161,987, filed Oct. 28, 1999; Ser. No. 09/697,297, filed Oct. 27, 2000; Ser. No. 09/995,659, filed Nov. 29, 2001; Ser. No. 10/374,512, filed Feb. 27, 2003; PCT application no. PCT/US/00/29551, filed Oct. 27, 2000; the disclosures of which are incorporated herein in their entirety).

The term “human embryonic stem cells” (hES cells) refers to human ES cells.

The term “human iPS cells” refers to cells with properties similar to hES cells, including the ability to form all three germ layers when transplanted into immunocompromised mice wherein said iPS cells are derived from cells of varied somatic cell lineages following exposure to de-differentiation factors, for example hES cell-specific transcription factor combinations: KLF4, SOX2, MYC, and OCT4 or SOX2, OCT4, NANOG, and LIN28. Any convenient combination of de-differentiation factors may be used to produce iPS cells. Said iPS cells may be produced by the expression of these genes through vectors such as retroviral, lentiviral or adenoviral vectors as is known in the art, or through the introduction of the factors as proteins, e.g., by permeabilization or other technologies. For descriptions of such exemplary methods see: PCT application number PCT/US2006/030632, filed on Aug. 3, 2006; U.S. application Ser. No. 11/989,988; PCT Application PCT/US2000/018063, filed on Jun. 30, 2000; U.S. Application Ser. No. 09,736,268 filed on Dec. 15, 2000; U.S. application Ser. No. 10/831,599, filed Apr. 23, 2004; and U.S. Patent Publication 20020142397 (application. Ser. No. 10/015,824, entitled “Methods for Altering Cell Fate”); U.S. Patent Publication 20050014258 (application. Ser. No. 10/910,156, entitled “Methods for Altering Cell Fate”); U.S. Patent Publication 20030046722 (application. Ser. No. 10/032,191, entitled “Methods for cloning mammals using reprogrammed donor chromatin or donor cells”); and U.S. Patent Publication 20060212952 (application. Ser. No. 11/439,788, entitled “Methods for cloning mammals using reprogrammed donor chromatin or donor cells”) all of which are incorporated herein by reference in their entirety.

The term “ICM cells” refers to the cells of the inner cell mass of a mammalian embryo or the cells of the inner cell mass cultured in vitro with or without the surrounding trophectodermal cells.

The term “oligoclonal” refers to a population of cells that originated from a small population of cells, typically 2-1000 cells, that appear to share similar characteristics such as morphology or the presence or absence of markers of differentiation that differ from those of other cells in the same culture. Oligoclonal cells are isolated from cells that do not share these common characteristics, and are allowed to proliferate, generating a population of cells that are essentially entirely derived from the original population of similar cells.

The term “organotypic culture” refers to cultured cells that are aggregated to create a three-dimensional structure with tissue-like cell density such as occurs in the culture of some cells over a layer of agar, cultured as teratomas in an animal, otherwise grown in a three dimensional culture system but wherein said aggregated cells contain cells of different cell lineages, such as, by way of nonlimiting examples, the combination of epidermal keratinocytes and dermal fibroblasts, or the combination of parenchymal cells with their corresponding tissue stroma, or epithelial cells with mesenchymal cells.

The term “pluripotent stem cells” refers to animal cells capable of differentiating into more than one differentiated cell type. Such cells include hES cells, blastomere/morula cells and their derived hED cells, hiPS cells, hEG cells, hEC cells, and adult-derived cells including mesenchymal stein cells, neuronal stem cells, and bone marrow-derived stein cells. Pluripotent stem cells may be genetically modified or not genetically modified. Genetically modified cells may include markers such as fluorescent proteins to facilitate their identification within the egg.

The term “pooled clonal” refers to a population of cells obtained by combining two or more clonal populations to generate a population of cells with a uniformity of markers such as markers of gene expression, similar to a clonal population, but not a population wherein all the cells were derived from the same original clone. Said pooled clonal lines may include cells of a single or mixed genotypes. Pooled clonal lines are especially useful in the cases where clonal lines differentiate relatively early or alter in an undesirable way early in their proliferative lifespan.

The term “primordial stem cells” refers to animal cells capable of differentiating into more than one differentiated cell type. Such cells include hES cells, blastomere/morula cells and their derived hED cells, hiPS cells, hEG cells, hEC cells, and adult-derived cells including mesenchymal stein cells, neuronal stem cells, and bone marrow-derived stem cells. Primordial stem cells may be genetically modified or not genetically modified. Genetically modified cells may include markers such as fluorescent proteins to facilitate their identification in vitro or in vivo.

The term “substantially the same,” as it is used herein to describe the genomes or 2 cells or 2 cell lines, e.g. a clonal progenitor cell line and its parental pluripotent stein cell line such as an hES cell line or an iPS cell line, means that the genomes of the two cells or cell lines are about 95% identical, about 96% identical, about 97% identical, about 98% identical, about 99% identical. In some embodiments the genomes of the two cells or cell lines may be greater than 95% identical, greater than 96% identical, greater than 97% identical, greater than 98% identical, greater than 99% identical.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Methods

In addition to the methods described below, methods that find use in the production and use of the cell lines described herein can be found in the following: U.S. Patent Publication 20080070303, entitled “Methods to accelerate the isolation of novel cell strains from pluripotent stem cells and cells obtained thereby”; U.S. patent application Ser. No. 12/504,630 filed on Jul. 16, 2009 and titled “Methods to Accelerate the Isolation of Novel Cell Strains from Pluripotent Stem Cells and Cells Obtained Thereby”; U.S. provisional application Ser. No. 61/226,237 filed on Jul. 16, 2009 and titled “Methods and Compositions Useful for In Vitro and In Vivo Chondrogenesis Using Embryonic Progenitor Cell Lines”; PCT Application PCT/US2006/013519, filed on Apr. 11, 2006, entitled “NOVEL USES OF CELLS WITH PRENATAL PATTERNS OF GENE EXPRESSION”; and U.S. patent application Ser. No. 13/115,688 filed on May 25, 2011 and titled “Improved Methods of Screening Embryonic Progenitor Cell Lines” each of which is incorporated by reference herein in its entirety.

### hES Cell Culture and Generation of Candidate Cultures.

The hES cell line ESI-017 used herein was previously described (Crook et al, (2007) The Generation of Six Clinical-Grade Human Embryonic Stem Cell Lines, Cell Stem Cell 1: 490-494). hES cells were routinely cultured in hES medium (KO-DMEM (Invitrogen, Carlsbad, Calif.), IX nonessential amino acids (Invitrogen, Carlsbad, Calif.), 1× Glutamax-1 (Invitrogen, Carlsbad, Calif.), 55 uM beta-mercaptoethanol (Invitrogen, Carlsbad, Calif.), 8% Knock-Out Serum Replacement (Invitrogen, Carlsbad, Calif.), 8% Plasmanate, 10 ng/ml LIF (Millipore, Billerica, Mass.), 4 ng/ml bFGF (Millipore, Billerica, Mass.), 50 unit/ml Penicillin—50 units/ml Streptomycin (Invitrogen, Carlsbad, Calif.). The hES cell lines were maintained at 37 deg C in an atmosphere of 10% CO2 and 5% O2 on Mitomycin-C treated mouse embryonic fibroblasts (MEFs) and passaged by trypsinization or periodic manual selection of colonies. For the production of clonal embryonic progenitors, hES cells were plated at 500-10,000 cells per 15 cm dish and then differentiated under a two-step protocol, the first step being the differentiation of hES cells under an array of conditions to yield diverse heterogeneous cultures of cells called “candidate cultures.” The generation of candidate cultures may be performed with either adherent hES cells grown on MEFs (colony in situ differentiation) or with hES-derived embryoid bodies (EB). For colony in situ differentiation experiments, hES cells may be allowed to grow to confluence and differentiated by a variety of methods (as described in Supplementary Table I from West et al., 2008, Regenerative Medicine vol. 3(3) pp. 287-308, which is incorporated by reference herein in its entirety). By way of nonlimiting example, in the case of colony in situ differentiation in DMEM with 10% FCS, culture medium may be aspirated from cultures of hES cell colonies on mouse feeders, and the media was replaced with DMEM medium containing 10% FBS for differentiation and after various time periods (1, 2, 3, 4, 5, 7, and 9 days in differentiation medium). The cells may be dissociated with 0.25% trypsin (Invitrogen, Carlsbad, Calif.) and plated in 150 cm² flasks for expansion. The candidate cells from each time point in the 150 cm² flasks may be plated out for cloning and expansion as described below. For EB differentiation experiments, confluent hES cultures may be treated for 15 minutes at 37 deg C with 1 mg/ml Collagenase IV (in DMEM, Invitrogen, Carlsbad, Calif.) to release the colonies. The detached, intact colonies may be scraped and collected by centrifugation (150×g for 5 minutes), resuspended in differentiation medium described in Supplementary Table I (from West et al., 2008, Regenerative Medicine vol. 3(3) pp. 287-308, which is incorporated by reference herein in its entirety) and transferred to a single well of a 6-well Ultra-Low Binding plate (Corning, distributed by Fisher Scientific, Pittsburgh, Pa.) containing the same differentiation medium. The EBs may be allowed to differentiate, depending on the experiment, from 4-7 days and the differentiated EBs dissociated with 0.25% trypsin, plated in 6-well plates containing various expansion medium. The candidate cultures in the 6 well plates may be allowed to grow to confluence and plated out for cloning and expansion as described below.

### Isolation and Expansion of Clonal Cell Lines.

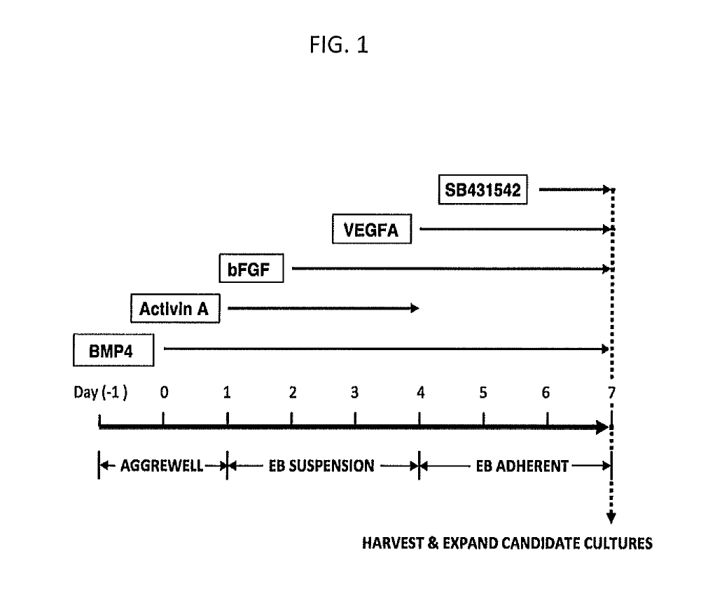

The partially differentiated candidate cell cultures described above may be dissociated with 0.25% trypsin to single cells and plated onto duplicate 15 cm gelatin or Matrigel-coated plates at cloning densities of approximately 500 and/or 1,000 and/or 2,000 and/or 5,000 cells per plate for further differentiation and expansion in a variety of growth media shown in Supplementary Table I (from West et al., 2008, Regenerative Medicine vol. 3(3) pp. 287-308, which is incorporated by reference herein in its entirety). The clonal density cells may be allowed to grow, undisturbed, for 10-14 days and colonies that develop may be identified and collected with cloning cylinders and trypsin using standard techniques. The cloned colonies may be transferred onto gelatin or Matrigel-coated 24 well plates for expansion. As the clones become confluent in the 24 well plates (but without letting the cells remain confluent for more than 2 days), they may be sequentially expanded to gelatin or Matrigel-coated 12 well, 6 well, T-25 flask, T-75 flask, T-150 or 1-225 flasks and, finally, roller bottles. Clonal cell lines that expand to the roller bottle stage may be photographed and cryopreserved in aliquots for later use. Once cells reached a confluent 6 well dish, they may be passaged to a T-25 flask and a fraction of the cells (5×10⁵) were removed for plating in a gelatinized or Matrigel-coated 6 cm dish for gene expression profile analysis. Alternatively, some cells may be first passaged to T-225 flasks, then a fraction of the cells (5×10⁵) may be removed for plating in a gelatinized or Matrigel-coated 6 cm dish for gene expression profile analysis. The population doublings that the cells had undergone were therefore determined to be approximately 26PDs. Cell Culture media utilized in experiments following the initial differentiation occurring most preferably for 1-7 days or preferably 1-30 days, such as that outlined in FIG. 1 includes an inhibitor specific for the type I TGFβ receptors. In some embodiments, the inhibitor is an inhibitor of ALK4, ALK5, and ALK7. In other embodiments, the inhibitor is an inhibitor of at least ALK5. In one embodiment, the TGFβ signaling inhibitor is a soluble form of a type I receptor, an antibody directed to a type I receptor, or a small molecule compound. In specific embodiments, the inhibitor is a small compound selected from SB-431542, A 83-01, D 4476, LY 364947, SB 525334, SD 208, and SJN 2511. Such basal media to which the inhibitor specific for the type I TGFβ receptors is added may include: Smooth muscle cell basal medium (Cat# C-22062B) and growth supplement (Cat# C-39267), Skeletal muscle basal medium (Cat# C-22060B) and growth supplement (Cat# C-39365), Endothelial cell basal medium (Cat# C-22221) and growth supplement (Cat# C-39221), Melanocyte cell basal medium (Cat# C-24010B) and growth supplement (Cat# C-39415) were obtained from PromoCell GmbH (Heidelberg, Germany). Epi-Life, calcium free/phenol red free medium (Cat# M-EPIcf/PRF-500) and low serum growth supplement (Cat# S-003-10) were purchased from Cascade Biologics (Portland, Oreg.). Mesencult basal medium (Cat#05041) and supplement (Cat#5402) were obtained from Stem Cell Technologies (Vancouver, BC). Dulbecco's modified Eagle's medium (Cat#11960-069) and Fetal bovine serum (Cat# SH30070-03) were purchased from Invitrogen (Carlsbad, Calif.) and Hyclone (Logan, Utah) respectively. Medium and supplements were combined according to manufacturer's instructions.

### Clonal Embryonic Progenitor Line Nomenclature:

The cell lines of the present invention along with their alternative designations are provided infra. Synonyms that represent minor modifications that result from the manipulation of the names resulting from bioinformatics analysis, including the substitution of “-” for “.” and vice versa, the inclusion of an “x” before cell line names beginning with an arabic number, and suffixes such as “bio1” or “bio2” that indicate biological replicates of the same line which are examples of cases where a frozen ampule of the same line was thawed, propagated, and used in a parallel analysis and “Rep1” or “Rep2” which indicate technical replicates wherein RNA isolated from a given cell line is utilized a second time for a repeat analysis without thawing or otherwise beginning with a new culture of cells. Passage number (which is the number of times the cells have been trypsinized and replated) for the cell lines is usually designated by the letter “P” followed by an arabic number, and in contrast, the population doubling number (which refers to the number of estimated doublings the cell lines have undergone in clonal expansion from one cell) is designated by the letters “PD” followed by an arabic number. The number of PDs in a passage varied from experiment to experiment but generally each trypsinization and replating was at a 1:3 to 1:4 ratio (corresponding to an increase of PDs of 1.5 and 2 respectively). In the expansion of clones, the original colonies were removed from tissue culture plates with cloning cylinders, and transferred to 24-well plates, then 12-well, and 6-well as described above. First confluent 24 well is designated P1, the first confluent 12 well culture is P2, the first 6-well culture is P3, then the six well culture was then split into a second 6 well plate (P4) and a T25 (P4). The cells at P6 are utilized for RNA extraction (see U.S. patent application Ser. No. 12/504,630 filed on Jul. 16, 2009 and titled “Methods to Accelerate the Isolation of Novel Cell Strains from Pluripotent Stem Cells and Cells Obtained Thereby”, incorporated herein by reference in its entirety) and represents about 26 PD of clonal expansion. Typical estimated subsequent passages and PDs are the following split to a T75 flask (19.5-215 PD), the P6 passage of the cells to a T225 flask (21-24 PD), then P7 being the transfer of the cells to a roller bottle (850 cm², 23-26 PD), and P8 the split into 4 rollers (25-28 PD). The ranges shown above in parenthesis represent estimated ranges in cell counts due to cell sizes, attachment efficiency, and counting error.

Cells lines with the MV2 prefix in their name are grown on Matrigel in MV2 Promocell endothelial media (Promocell, Heidelberg, Germany) and those with SK in the name are grown on Matrigel in Promocell skeletal muscle media (Promocell, Heidelberg, Germany).

### Propagation of Clonal, Pooled Clonal, Oligoclonal, and Pooled Oligoclonal Cell Lines.

Aspects of the invention provide methods for identifying and differentiating embryonic progenitor cell lines that are derived from a single cell (clonal) or cell lines that are “pooled clonal” meaning that cell lines cloned have indistinguishable markers, such as gene expression markers, and are combined to produce a single cell culture often for the purpose of increasing the number of cells in a culture, or are oligoclonal wherein a line is produced from a small number, typically 2-1,000 similar cells and expanded as a cell line, or “pooled oligoclonal” lines which are lines produced by combining two or more oligoclonal cell lines that have indistinguishable markers such as patterns of gene expression. Said clonal, pooled clonal, oligoclonal, or pooled oligoclonal cell lines are then propagated in vitro through removal of the cells from the substrate to which they are affixed, and the replating of the cells at a reduced density of typically ⅓ to ¼ of the original number of cells, to facilitate further proliferation. Examples of said cell lines and their associated cell culture media is disclosed in U.S. patent application Ser. No. 12/504,630 filed on Jul. 16, 2009 and titled “Methods to Accelerate the Isolation of Novel Cell Strains from Pluripotent Stem Cells and Cells Obtained Thereby”; and West et al., 2008, Regenerative Medicine vol. 3(3) pp. 287-308, both of which are incorporated herein by reference, including supplemental information. The compositions and methods of the present invention relate to said cell fines cultured as described but for greater than 21 doublings of clonal expansion.

### Gene Expression Analysis

To reduce variations in gene expression due to cell cycle artifacts, and to capture an early gene expression profile of the cells, upon being expanded to six well plates, on the day the cells reached confluence, the cells were placed in media with a reduction of serum to 0.5% in the case where the original serum concentration was >5%. In all other cases, serum and/or other growth factors was reduced to 10% of their original values. These quiescence conditions were imposed for five days and all cultures were re-fed two days prior to harvest to reduce feeding difference artifacts. So, by way of example, if the original media was DMEM medium with 10% FCS, then the quiescence synchronization media was DMEM with 0.5% FCS. Total RNA was extracted directly from cells growing in 6-well or 6 cm tissue culture plates using Qiagen Rneasy mini kits according to the manufacturer's instructions. RNA concentrations were measured using a Beckman DU530 or Nanodrop spectrophotometer and RNA quality determined by denaturing agarose gel electrophoresis or an Agilent 2100 bioanalyzer. Whole-genome expression analysis was carried out using Illumina Beadchips, and RNA levels for certain genes were confirmed by quantitative PCR. For Illumina BeadArrays, total RNA was linearly amplified and biotin-labeled using Illumina TotalPrep kits (Ambion), and cRNA was quality controlled using an Agilent 2100 Bioanalyzer. cRNA was hybridized to Illumina BeadChips, processed, and read using a BeadStation array reader according to the manufacturer's instructions (Illumina). Relative Fluorescence Unit (RFU) values for all of the cell lines with common probe sets were quantile normalized.

Low Throughput Screening and qPCR

The clonal, oligoclonal, or pooled clonal or pooled oligoclonal embryonic progenitor cell lines of the present invention at either <21 or preferably >21 doublings of clonal or oligoclonal expansion, most preferably at 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 doublings of clonal expansion (since before 29 doublings of clonal expansion the cells are available only in limited quantities, and beyond 70 doublings the cells normally approach senescence) are screened simultaneously in 1, 2, 3, 4, 5, or preferably 10 or more diverse differentiation conditions. Said differentiation conditions may include without limitation, all combinations of the human embryonic progenitor cell lines disclosed infra, together with culture conditions as listed in Table I, exposed to the culture media listed in Table II, and supplemented factors listed in Table III. The cells may be cultured in said differentiation conditions for 1-6 weeks, e.g. two to four weeks.

The readout of the assay can be mRNA markers of differentiation detected by PCR, gene expression microarrays, or RNA sequencing. Detection can also be at the level of peptides or proteins that may be detected through the use of specific antibodies, through the use of enzyme assays, mass spectroscopy, or other similar means well known in the art.

In the case of qPCR, protocols may vary and are well-known in the art. By way of nonlimiting example, samples for testing are prepared in standard Optical 96-well reaction plates (Applied Biosystems Carlsbad, Calif., PN 4306737) consisting of 30 ng of RNA equivalent of cDNA, 0.4 uM per primer, Ultra-Pure distilled water (Invitrogen), diluted 1:1 with 12.5 ul of Power SYBR Green PCR Master Mix (Applied Biosystems Carlsbad, Calif., Cat#4367659) incorporating AmpliTaq Gold DNA polymerase in a total reaction volume of 25 ul. Real-Time qPCR is run using Applied Biosystems 7500 Real-Time PCR System employing SDSv1.2 software. Amplification conditions are set at 50° C. for 2 min. (stage 1), 95° C. for 10 min. (stage 2), 40 cycles of 95° C. for 15 sec then 60° C. for 1 min (stage 3), with a dissociation stage at 95° C. for 15 sec, 60° C. for 1 min, and 95° C. for 15 sec (stage 4). Ct values for amplification products of genes of interest are normalized to the average Ct value of 3 housekeeping genes (GAPD, RPS10, and GUSB).

### Medium Throughput Screen of the Fate Space of Clonal or Oligoclonal Embryonic Progenitors.

The clonal, oligoclonal, or pooled clonal or pooled oligoclonal embryonic progenitor cell lines of the present invention at either <21 or >21 doublings of clonal or oligoclonal expansion, e.g. at 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 doublings of clonal expansion (since before 29 doublings of clonal expansion the cells are available only in limited quantities, and beyond 70 doublings the cells normally approach senescence) may be screened simultaneously in 10, 20, 30, 40, 50, or preferably 100 or more diverse differentiation conditions. Said differentiation conditions may include without limitation, all combinations of the human embryonic progenitor cell lines disclosed infra, together with culture conditions that include BMP family members including TGFB1, TGFB2, TGFB3, BMP2, BMP4 (1-100 ng/mL, preferably 10 ng/mL), BMP6 (3-300 ng/mL, preferably 30 ng/mL), BMP7 (10-1,000 ng/mL, preferably 100 ng/mL), and GDF5 (10-1,000 ng/mL, preferably 100 ng/mL) or combinations of these BMP family members. The cells are cultured in said differentiation conditions for 1-6 weeks, e.g. two weeks.

The readout of the assay can be mRNA markers of differentiation such as those listed in Table IV and measured by hybridization to arrayed target sequences, including but not limited to microarrays or PCR. Detection can also be at the level of peptides or proteins that may be detected through the use of specific antibodies, through the use of enzyme assays, mass spectroscopy, or other similar means well known in the art.

Medium Throughput qPCR Screen of hEP Cell Differentiation

The clonal, oligoclonal, or pooled clonal or pooled oligoclonal embryonic progenitor cell lines of the present invention, including but not limited to those disclosed infra, at either <21 or >21 doublings of clonal or oligoclonal expansion, most preferably at 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 doublings of clonal expansion are plated in 6 well culture plates with each well having 10 micromasses of 250,000 cells (i.e. 2.5 million cells per well). Alternatively the cells are treated with other culture conditions as listed in Table 1 using the same number of cells, exposed to any combination of the culture media listed in Table H, and supplemented factors listed in Table III or detailed protocols listed in Table V. The cells are cultured in said differentiation conditions for 1-6 weeks, e.g. four weeks.

RNA is prepared from cell lysates using the Rneasy mini kits (Qiagen) according to the manufacturer's instructions. Briefly, cell cultures (micromasses) are rinsed in PBS, then lysed in a minimal volume of the RLT lysis buffer. After incubation on ice, the cell debris is removed by centrifugation and the lysate is mixed with RLT buffer, after which ethanol is added to the mixture. The combined mixture is then loaded onto the Rneasy spin column and centrifuged; the loaded column is then washed and the purified RNA is released from the column with a minimal volume of DEPC-treated water (typically 30 ul or less). The concentration of RNA in the final eluate is determined by absorbance at 260 nm.

cDNA synthesis is performed using the SuperScript First Strand cDNA kit (InVitrogen; Carlsbad, Calif.). Briefly, 2.5 ug of purified RNA is heat denatured in the presence of random hexamers. After cooling, the first strand reaction is completed using SuperSript reverse transcriptase enzyme and associated reagents from the kit. The resulting product is further purified using QIAquick PCR Purification kits (Qiagen) according to the manufacturer's instructions. Briefly, PB buffer is added to the first strand cDNA reaction products, then the mixture is loaded onto the QIAquick spin column and centrifuged. The column is washed with PE buffer and the purified cDNA is eluted from the column using a minimal volume of water (20 ul).

qPCR primer pairs are synthesized for each target gene. Briefly, primer pairs for a target gene are designed to amplify only the target mRNA sequence and optimally have annealing temperatures for their target sequences that lie in the range of 65-80° C. and unique amplification products in the size range of 100-500 bp. Primer pairs are supplied at working concentrations (10 uM) to BioTrove, Inc. (Woburn, Mass.) for production of a custom qPCR Open Array plate. OpenArray plates are designed to accommodate 56-336 primer pairs and the final manufactured plate with dried down primer pairs is provided to the service provider. Purified cDNA reaction products (2.) and Syber green master mix are loaded into individual wells of the OpenArray plate using OpenArray autolader device (BioTrove). The plate is sealed and the qPCR and loaded into the NT Imager/Cycler device (BioTrove) for amplification. Ct values for each sample are calculated using the OpenArray application software.

Markers of differentiation are not those present in embryonic progenitor cell lines, but are present in later stages of differentiation. It is not obvious to what an effective array of such markers would be. For example, COL2A1 is not expressed in the clonal embryonic progenitor cell lines, but is markedly induced >100-fold in a subset of the cell lines of the present invention. Previous attempts to invent an array of differentiation markers were not useful in the context of the present invention because they included a majority of markers that were expressed in both embryonic progenitor cell types and in terminally-differentiated cell types (Luo, Y., Ginis, L, Sun, Y., Lee, S., Yu, S. X., Hoke, A., and Rao, M. 2003, Stem Cells 21:575). An example of a list of said markers useful in determining that a particular differentiation condition induced terminal differentiation in embryonic progenitor cell lines a majority of which are not expressed in embryonic progenitor cell lines are shown in Table IV.

### Isolation of Secreted or Extracellular Matrix Proteins

Secreted Protein Isolation Protocol 1—Conditioned Medium

Cells were grown in either their normal propagation medium (West et al., 2008, Regen Med vol. 3(3) pp. 287-308) or the differentiation conditions described herein. To obtain conditioned medium on a smaller scale (typically 1-2 L or less), the cells were grown in monolayer cultures in T150, T175 or T225 flasks (Corning or BD Falcon) in a 37° C. incubator with 10% CO₂ atmosphere. For larger volume medium collections, the cells were typically grown either in 2 L roller bottles, on microcarrier suspensions (porous such as Cytodex varieties from Sigma-Aldrich, St. Louis, Mo., or non-porous such as from SoloHill Engineering, Ann Arbor, Mich.) in spinner flasks or other bioreactors, or in hollow fiber cartridge bioreactors (GE Healthcare, Piscataway, N.J.). Prior to conditioned medium collection, the cultures were rinsed twice with PBS and then incubated for 2 hours at 37° C. in the presence of serum-free medium wherein the medium is the same basal medium as described herein for the propagation or differentiation of the cells, in order to remove fetal serum proteins. The serum-free medium was then removed and replaced with fresh medium, followed by continued as described herein at 37° C. for 24-48 hours.

The culture-conditioned medium was then collected by separation from the cell-bound vessel surface or matrix (e.g., by pouring off directly or after sedimentation) and processed further for secreted protein concentration, enrichment or purification. As deemed appropriate for the collection volume, the culture medium was first centrifuged at 500 to 10,000×g to remove residual cells and cellular debris in 15 or 50 ml centrifuge tubes or 250 ml bottles. It was then passaged through successive 1 μm or 0.45 μm and 0.2 μm filter units (Corning) to remove additional debris, and then concentrated using 10,000 MW cutoff ultrafiltration in a stirred cell or Centricon centrifuge filter (Amicon-Millipore) for smaller volumes, or using a tangential flow ultrafiltration unit (Amicon-Millipore) for larger volumes. The retained protein concentrate was then dialyzed into an appropriate buffer for subsequent purification of specific proteins, and further purified using a combination of isoelectric focusing, size exclusion chromatography, ion exchange chromatography, hydrophobic or reverse phase chromatography, antibody affinity chromatography or other well-known methods appropriate for the specific proteins. During the various steps in the purification process, collection fractions were tested for the presence and quantity of the specific secreted protein by ELISA (e.g., using BMP-2 or BMP-7 ELISA kits from R&D Systems, Minneapolis, Minn.). The purified proteins were then kept in solution or lyophilized and then stored at 4 or minus 20-80° C.

Secreted Protein Isolation Protocol 2—Urea-Mediated Protein Extraction

In the case of some secreted proteins, interactions with the cell or ECM components may reduce the simple diffusion of factors into the medium as described above in Secreted Protein Isolation Protocol 1. A simple comparison of the yield in the two protocols will suffice to determine which protocol provides the highest yield of the desired factors. In the case of Secreted Protein Isolation Protocol 2, a low concentration of urea is added to facilitate the removal of factors. In the case of the examples provided, all urea extractions were performed two days subsequent to feeding. On the second day, cell monolayers in T-150 cell culture flasks were rinsed twice with CMF-PBS and then incubated for two hours at 37° C. in the presence of scrum-free medium. The rinse with CMF-PBS and the incubation in serum-free medium together aid in the removal of fetal serum proteins from the surface of the cells. The serum-free medium was then removed and 10 ml/T150 of freshly made 200 mM urea in CMF-PBS was added. The flasks were then placed on a rocker at 37° C. for 6.0 hours. The urea solution was then removed and immediately frozen at −70° C.

Extracellular Matrix Isolation Protocol 1—DOC-Mediated Preparation

Extracellular matrix proteins can be extracted using the method of Hedman et al, 1979 (Isolation of the pericellular matrix of human fibroblast cultures. J. Cell Biol. 81: 83-91). Cell layers are rinsed three times with CMF-PBS buffer at ambient temperature and then washed with 30 mL of 0.5% sodium deoxycholate (DOC), 1 mM phenylmethylsulfonylfluride (PMSF, from 0.4M solution in EtOH), CMF-PBS buffer 3×10 min. on ice while on a rocking platform. The flasks were then washed in the same manner with 2 mM Tris-HCl, pH 8.0 and 1 mM PMSF 3×5 min. The protein remaining attached to the flask was then removed in 2 mL of gel loading buffer with a rubber policeman.

### Screening of Secreted or Extracellular Matrix Proteins for Biological Activity

The cell lines of the present invention are also useful as a means of screening diverse embryonic secretomes for varied biological activities. The cell lines of the present invention cultured at 18-21 doublings of clonal expansion express a wide array of secreted soluble and extracellular matrix genes (see US Patent Application Publication 2010/0184033 entitled “METHODS TO ACCELERATE THE ISOLATION OF NOVEL CELL STRAINS FROM PLURIPOTENT STEM CELLS AND CELLS OBTAINED THEREBY” filed on Jul. 16, 2009, incorporated herein by reference). These proteins, proteoglycans, cytokines, and growth factors may be harvested from the cell lines of the present invention by various techniques known in the art including but not limited to Secreted Protein Isolation Protocol 1 or 2. These pools of secreted and extracellular matrix proteins may be further purified or used as mixtures of factors and used in varied in vitro or in vivo assays of biological activity as is known in the art.

### Applications

The disclosed methods for the culture of animal cells and tissues are useful in generating cells or progeny thereof in mammalian and human cell therapy, such as, but not limited to, generating human cells useful in treating vascular and muscle disorders in humans and nonhuman animals.

In certain embodiments of the invention, single cell-derived and oligoclonal cell-derived cells derived by methods of this invention, are utilized in research and treatment of disorders relating to cell biology, cell-based drug discovery and in cell therapy. The single cell-derived cell populations derived using the methods of the present invention may already have received the requisite signals to be directed down a differentiation pathway such as a commitment to only endodermal, mesodermal, ectodermal, or neural crest lineages. For example, some mesodermal cells may express genes consistent with vascular endothelial or skeletal muscle gene expression, in particular, a prenatal pattern of gene expression useful in promoting angiogenesis or regenerating injured or diseased skeletal or cardiac muscle.

The vascular endothelial cells of the present invention may or may not be genetically modified. In the case of the unmodified cells, the cells may be introduced into a tissue by: 1) Direct injection in vivo as described herein; 2) By combination with diverse nonvascular somatic cell types to improve engraftment of said non-vascular somatic cell type in humans or nonhuman animals; 3) Utilized in the targeting and destruction of tumor vasculature; 4) Used to generate tissue engineered blood or lymphatic vessels; or 5) Combined with diverse somatic cell types and extracellular matrices to manufacture vascularized tissue engineered organoids, organs, or other three dimensional aggregates.

Direct injection in vivo. In certain embodiments of the invention, single cell-derived and oligoclonal cell-derived vascular endothelial cells are introduced into tissues in vivo in order to increase circulation in the target tissue and thereby impart a therapeutic utility. Such cells include, for example, vascular endothelial cells such as those with specific patterns of gene expression specific to the brain, cardiac, hepatic, renal glomerulus, osteogenic, neuronal, lung, pancreatic, intestinal heart, arteries, veins, lymphatics, liver, microvessels specific to diverse tissues, capillaries, venules, arterioles, and endothelial cells suitable for targeting to and delivering a toxic payload to malignant tumors. Said vascular endothelial cells may also be formulated with diverse cell types such as, and other diverse somatic cell types to improve vascularization after engraftment into humans and nonhuman animals.

Combination of the endothelial cells of the present invention with diverse nonvascular somatic cell types to improve engraftment of said non-vascular somatic cell type in humans or nonhuman animals. The endothelial cells may be mixed as a disaggregated slurry of individual cells with cells of other cell types and injected into diverse tissue types to promote the engraftment and vascularization of the engrafted cells. In addition to the combination of these two cell types, matrices such as HyStem hydrogels (described herein) may be used to improve the survival and engraftment of both cell types.

Utilization in the targeting and destruction of tumor vasculature. The vascular endothelial cells of either arterial, venous, or lymphatic phenotypes may be genetically modified and used to deliver a toxic payload or clotting factors to the vasculature or stroma of malignant tumors including tumors of the liver wherein the cells are introduced into the hepatic artery (see, e.g., PCT application Ser. No. PCT/US03/01827 filed on Jan. 22, 2003 titled “STEM CELL-DERIVED ENDOTHELIAL CELLS MODIFIED TO DISRUPT TUMOR ANGIOGENESIS” and published as WO 2003/061591, incorporated herein by reference).

Use of endothelial cells to generate tissue engineered blood or lymphatic vessels. The diverse types of endothelial cells described herein may be used in the generation of tissue engineered blood vessels including arteries, veins, and lymphatic vessels as is known in the art.

Use of hemogenic endothelium for the production of hematopoietic cells, Preferably CD34+ endothelial cells of the present invention, and most preferably HOXB4+ endothelial cells may be utilized as a source of hematopoietic cells by culture of the cells in the presence of cytokines known in the art as stimulating hematopoiesis (see U.S. patent application Ser. No. 12/991,096, filed on May 6, 2009, published as US 2011/0064705 and titled “HEMANGIO COLONY FORMING CELLS AND NON-ENGRAFTING HEMANGIO CELLS”, incorporated herein by reference).

Examples of genetic modifications introduced in the vascular endothelial cells of the present invention are genetic modifications engineered into pluripotent stem cells including but not limited to hES and hiPS cells such that the endothelial cell lines produced from them express secreted factors useful in preventing thrombus formation in the vessels that contain the cells or increased the angiogenic capacity of said cells in tissues or tissue engineered constructs. Additional examples of genetic modifications introduced into pluripotent stem cells to increased the usefulness of derived endothelial cells is the overexpression of the erythropoietin receptor to increase endothelial proliferation and survival and overexpression of eNOS wherein the cells are used to treat coronary disease (Dzau V. et al, Hypertention 46: 7-18 (2005)) incorporated herein by reference.

The skeletal muscle myoblast cells of the present invention such as the cell lines RP1-SKEL-8 or RP1-SM2-12 or cell lines with a gene expression pattern similar to or matching that shown for those cell lines disclosed infra may or may not be genetically modified. Examples of genetic modifications introduced in the skeletal muscle myoblast cells of the present invention are genetic modifications engineered into pluripotent stem cells including but not limited to hES and hiPS cells are targeted genetic modifications to correct inherited genetic defects of skeletal muscle including but not limited to Duchenne muscular dystrophy, and other muscular dystrophies. Additional genetic modifications are the expression of Connexin-43 (GJA1 also known as CX43) to reduce the incidence of arrythmias normally induced by the introduction of skeletal muscle cells into the heart (Abraham, M. R. 2005. Antiarrhythmic engineering of skeletal myoblasts for cardiac transplantation. Circ. Res. 97(2):159-67 incorporated herein by reference). Other useful genetic modifications are the introduction of transgenes to express secreted factors such as Factor IX for the treatment of hemophilia, and growth hormone or IGF-1 for the treatment of dwarfism or to increase muscle mass in aging (Subramanian I.V. et al, 2009. AAV-2-mediated expression of IGF-1 in skeletal myoblasts stimulates angiogenesis and cell survival. J. Cardiovasc. Transl. Res. 2(0:81-92 incorporated herein by reference). Other useful genetic modifications include the knockout of the myostatin (MSTN) gene or reduction of the expression of the AIM gene to increase resting muscle mass.

In certain embodiments of the invention, single cell-derived and oligoclonal cell-derived cells, derived by methods of this invention, are utilized in inducing the differentiation of other pluripotent stem cells. The generation of single cell-derived populations of cells capable of being propagated in vitro while maintaining an embryonic pattern of gene expression is useful in inducing the differentiation of other pluripotent stem cells. Cell-cell induction is a common means of directing differentiation in the early embryo. Many potentially medically-useful cell types are influenced by inductive signals during normal embryonic development, including spinal cord neurons, cardiac cells, pancreatic beta cells, and definitive hematopoietic cells. Single cell-derived populations of cells capable of being propagated in vitro while maintaining an embryonic pattern of gene expression can be cultured in a variety of in vitro, in ovo, or in vivo culture conditions to induce the differentiation of other pluripotent stem cells to become desired cell or tissue types. Induction may be carried out in a variety of methods that juxtapose the inducer cell with the target cell. By way of nonlimiting examples, the inducer cells may be plated in tissue culture and treated with mitomycin C or radiation to prevent the cells from replicating further. The target cells are then plated on top of the mitotically-inactivated inducer cells. Alternatively, single cell-derived inducer cells may be cultured on a removable membrane from a larger culture of cells or from an original single cell-derived colony and the target cells may be plated on top of the inducer cells or a separate membrane covered with target cells may be juxtaposed so as to sandwich the two cell layers in direct contact. The resulting bilayer of cells may be cultured in vitro, transplanted into a SPF avian egg, or cultured in conditions to allow growth in three dimensions while being provided vascular support (see, for example, international patent publication number WO/2005/068610, published Jul. 28, 2005, the disclosure of which is hereby incorporated by reference). The inducer cells may also be from a source of pluripotent stem cells, including hES or hED cells, in which a suicide construct has been introduced such that the inducer cells can be removed at will. Cell types useful in single cell-derived and oligoclonal cell-derived induction may include cases of induction well known in the art to occur naturally in normal embryonic development. In certain embodiments of the invention, single cell-derived cells and oligoclonal cell-derived cells, derived by methods of this invention, are used as “feeder cells” to support the growth of other cell types, including pluripotent stem cells. The use of single cell-derived cells and oligoclonal cell-derived cells of the present invention as feeder cells alleviates the potential risk of transmitting pathogens from feeder cells derived from other mammalian sources to the target cells. The feeder cells may be inactivated, for example, by gamma ray irradiation or by treatment with mitomycin C, to limit replication and then co-cultured with the pluripotent stem cells.

In certain embodiments of the invention, the extracellular matrix (ECM) of single cell-derived and oligoclonal cell-derived cells, derived by methods of this invention, may be used to support less differentiated cells (see Stojkovic et al., Stem Cells (2005) 23(3):306-14). Certain cell types that normally require a feeder layer can be supported in feeder-free culture on a matrix (Rosler et al., Dev Dyn. (2004) 229(2):259-74). The matrix can be deposited by preculturing and lysing a matrix-forming cell line (see WO 99/20741), such as the STO mouse fibroblast line (ATCC Accession No. CRL-1503), or human placental fibroblasts.

In certain embodiments of the invention, the conditioned media of single cell-derived and oligoclonal cell-derived cell cultures may be collected, pooled, filtered and stored as conditioned medium. This conditioned medium may be formulated and used for research and therapy. Such conditioned medium may contribute to maintaining a less differentiated state and allow propagation of cells such as pluripotent stem cells. In certain embodiments of the invention, conditioned medium of single cell-derived and oligoclonal cell-derived cell cultures derived by the methods of this invention can be used to induce differentiation of other cell types, including pluripotent stem cells. The use of conditioned medium of single cell-derived and oligoclonal cell-derived cell cultures may be advantageous in reducing the potential risk of exposing cultured cells to non-human animal pathogens derived from other mammalian sources (i.e. xenogeneic free).

In another embodiment of the invention, cell types that do not proliferate well under any known cell culture conditions may be induced to proliferate such that they can be isolated clonally or oligoclonally according to the methods of this invention through the regulated expression of factors that overcome inhibition of the cell cycle, such as regulated expression of SV40 virus large T-antigen (Tag), or regulated E1a and/or E1b, or papillomavirus E6 and/or E7, or CDK4 (see, e.g., U.S. patent application Ser. No. 11/604,047 filed on Nov. 21, 2006 and titled “Methods to Accelerate the Isolation of Novel Cell Strains from Pluripotent Stem Cells and Cells Obtained Thereby”, incorporated herein by reference).

In another embodiment of the invention, the factors that override cell cycle arrest may be fused with additional proteins or protein domains and delivered to the cells. For example, factors that override cell cycle arrest may be joined to a protein transduction domain (PTD). Protein transduction domains, covalently or non-covalently linked to factors that override cell cycle arrest, allow the translocation of said factors across the cell membranes so the protein may ultimately reach the nuclear compartments of the cells. PTDs that may be fused with factors that override cell cycle arrest include the PTD of the HIV transactivating protein (TAT) (Tat 47-57) (Schwarze and Dowdy 2000 Trends Pharmacol. Sci. 21: 45-48; Krosl et al. 2003 Nature Medicine (9): 1428-1432). For the HIV TAT protein, the amino acid sequence conferring membrane translocation activity corresponds to residues 47-57 (Ho et al., 2001, Cancer Research 61: 473-477; Vives et al., 1997, J. Biol. Chem. 272: 16010-16017). These residues alone can confer protein translocation activity.

In another embodiment of the invention, the PTD and the cycle arrest factor may be conjugated via a linker. The exact length and sequence of the linker and its orientation relative to the linked sequences may vary. The linker may comprise, for example, 2, 10, 20, 30, or more amino acids and may be selected based on desired properties such as solubility, length, steric separation, etc. In particular embodiments, the linker may comprise a functional sequence useful for the purification, detection, or modification, for example, of the fusion protein.

In another embodiment of the invention, single cell-derived or oligoclonal cell-derived cells of this invention may be reprogrammed to an undifferentiated state through novel reprogramming technique, as described in U.S. application No. 60/705,625, filed Aug. 3, 2005, U.S. application No. 60/729,173, filed Oct. 20, 2005; U.S. application No. 60/818,813, filed Jul. 5, 2006, the disclosures of which are incorporated herein by reference. Briefly, the cells may reprogrammed to an undifferentiated state using at least a two, preferably three-step process involving a first nuclear remodeling step, a second cellular reconstitution step, and finally, a third step in which the resulting colonies of cells arising from step two are characterized for the extent of reprogramming and for the normality of the karyotype and quality. In certain embodiments, the single cell-derived or oligoclonal cell-derived cells of this invention may be reprogrammed in the first nuclear remodeling step of the reprogramming process by remodeling the nuclear envelope and the chromatin of a differentiated cell to more closely resemble the molecular composition of an undifferentiated or a germ-line cell. In the second cellular reconstitution step of the reprogramming process, the nucleus, containing the remodeled nuclear envelope of step one, is then fused with a cytoplasmic bleb containing requisite mitotic apparatus which is capable, together with the transferred nucleus, of producing a population of undifferentiated stem cells such as ES or ED-like cells capable of proliferation. In the third step of the reprogramming process, colonies of cells arising from one or a number of cells resulting from step two are characterized for the extent of reprogramming and for the normality of the karyotype and colonies of a high quality are selected. While this third step is not required to successfully reprogram cells and is not necessary in some applications, the inclusion of the third quality control step is preferred when reprogrammed cells are used in certain applications such as human transplantation. Finally, colonies of reprogrammed cells that have a normal karyotype but not sufficient degree of programming may be recycled by repeating steps one and two or steps one through three.

In another embodiment of the invention, the single cell-derived and oligoclonal cell-derived cells may be used to generate ligands using phage display technology (see U.S. application No. 60/685,758, filed May 27, 2005, and PCT US2006/020552, filed May 26, 2006, the disclosures of which are hereby incorporated by reference).

In another embodiment of the invention, the single cell-derived or oligoclonal cell-derived cells of this invention may exhibit unique patterns of gene expression such as high levels of factors, e.g. secreted factors, that promote the development or formation of specific tissue types either in vitro or in vivo (e.g., angiogenic factors, neurotrophic factors, etc). Such cells may be useful for the delivery of these factors to tissues to promote the formation of specific cell/tissue types where those cells/tissues are therapeutic. For example, in the case of the angiogenic factors, cell lines that express high levels of such factors including VEGFA, B, C, or D or angiopoietin-1 or -2 can be transplanted using delivery technologies appropriate to the target tissue to deliver cells that express said angiogenic factor(s) to induce angiogenesis for therapeutic effect. In another embodiment of the invention, cells may produce large quantities of PTN (Accession number NM_{—}002825.5), MDK (Accession number NM_{—}002391.2), or ANGPT2 (Accession number NM_{—}001147.1), or other angiogenesis factors and therefore may be useful in inducing angiogenesis when injected in vivo as cell therapy, when mitotically inactivated and then injected in vivo, or when combined with a matrix in either a mitotically-inactivated or native state for use in inducing angiogenesis. PTN-producing cells described in the present invention are also useful when implanted in vivo in either a native or mitotically-inactivated state for delivering neuro-active factors, such as in preventing the apoptosis of neurons following injury to said neurons.

The expression of genes of the cells of this invention may be determined. Measurement of the gene expression levels may be performed by any known methods in the art, including but not limited to, microarray gene expression analysis, bead array gene expression analysis and Northern analysis. The gene expression levels may be represented as relative expression normalized to the ADPRT (Accession number NM_{—}001618.2), GAPD (Accession number NM_{—}002046.2), or other housekeeping genes known in the art. The gene expression data may also be normalized by a median of medians method. In this method, each array gives a different total intensity. Using the median value is a robust way of comparing cell lines (arrays) in an experiment. As an example, the median was found for each cell line and then the median of those medians became the value for normalization. The signal from the each cell line was made relative to each of the other cell lines.

In the case of angiogenesis, the senescence of the vascular endothelium or circulating endothelial precursor cells may blunt the response to angiogenic stimulus. The co-administration of young endothelial cells by various modalities known in the art based on the size of the animal and the target tissue along with cells capable of delivering an angiogenic stimulus will provide an improved angiogenic response. Such an induction of angiogenesis can be useful in promoting wound healing, the vascularization of tissues prone to ischemia such as aged myocardium, skeletal, or smooth muscle, skin (as in the case of nonhealing skin ulcers such as decubitus or stasis ulcers), intestine, kidney, liver, bone, or brain. Measurement of the gene expression levels may be performed by any known methods in the art, including but not limited to, microarray gene expression analysis, bead array gene expression analysis and Northern analysis. The gene expression levels may be represented as relative expression normalized to the ADPRT (Accession number NM_{—}001618.2), GAPD (Accession number NM_{—}002046.2), or other housekeeping genes known in the art. The gene expression data may also be normalized by a median of medians method. In this method, each array gives a different total intensity. Using the median value is a robust way of comparing cell lines (arrays) in an experiment. As an example, the median was found for each cell line and then the median of those medians became the value for normalization. The signal from the each cell line was made relative to each of the other cell lines.

In another embodiment of the invention, the single cell-derived or oligoclonal cell-derived cells of this invention may express unique patterns of CD antigen gene expression, which are cell surface antigens. The differential expression of CD antigens on the cell surface may be useful as a tool, for example, for sorting cells using commerically available antibodies, based upon which CD antigens are expressed by the cells. The mRNA expression profiles of CD antigens of the cells of this invention can be determined by examining the RFU values for the expression level of that particular gene.

In another embodiment of the invention, the single cell-derived and oligoclonal cell-derived cells, derived by methods of this invention, may be injected into mice to raise antibodies to differentiation antigens. Antibodies to differentiation antigens would be useful for both identifying the cells to document the purity of populations for cell therapies, for research in cell differentiation, as well as for documenting the presence and fate of the cells following transplantation. In general, the techniques for raising antibodies are well known in the art.

In another embodiment of the invention, the single cell-derived and oligoclonal cell-derived cells may be used for the purpose of generating increased quantities of diverse cell types with less pluripotentiality than the original stem cell type, but not yet fully differentiated cells. mRNA or miRNA can then be prepared from these cell lines and microarrays of their relative gene expression can be performed as described herein. In another embodiment of the invention, the single cell-derived and oligoclonal cell-derived cells may be used in animal transplant models, e.g. transplanting escalating doses of the cells with or without other molecules, such as ECM components, to determine whether the cells proliferate after transplantation, where they migrate to, and their long-term differentiated fate in safety studies.

In another embodiment of the invention, the single cell-derived and oligoclonal cell-derived cells generated according to the methods of the present invention are useful for harvesting mRNA, microRNA, and cDNA from either single cells or a small number of cells (i.e., clones) to generate a database of gene expression information. This database allows researchers to identify the identity of cell types by searching for which cell types in the database express or do not express genes at comparable levels of the cell type or cell types under investigation. For example, the relative expression of mRNA may be determined using microarray analysis as is well known in the art. The relative values may be imported into a software such as Microsoft Excel and gene expression values from the different cell lines normalized using various techniques well known in the art such as mean, mode, median, and quantile normalization. Hierarchical clustering with the single linkage method may be performed with the software such as The R Project for Statistical Computing as is well known in the art. An example of such documentation may be found at http(colon)//sekhon(dot)berkeley(dot)edu/stats/html/hclust.html. A hierarchical clustering analysis can then be performed as is well known in the art. These software programs perform a hierarchical cluster analysis using a group of dissimilarities for the number of objects being clustered. At first, each object is put in its own cluster, then iteratively, each similar cluster is joined until there is one cluster. Distances between clusters are computed by Lance-Williams dissimilarity update formula (Becker, R. A., Chambers, J. M. and Wilks, A. R. (1988) The New S Language. Wadsworth & Brooks/Cole. (S version.); Everitt, B. (1974). Cluster Analysis. London: Heinemann Educ. Books). Typically the vertical axis of the dendograms displays the extent of similarity of the gene expression profiles of the cell clones. That is, the farther down they branch apart, the more similar they are. The vertical axis is a set of n−1 non-decreasing real values. The clustering height is the value of the criterion associated with the clustering method for the particular agglomeration. In order to determine if a new cell line is identical to existing cell lines, two types of replicates are performed: biological and technical replicates. Biological replicates require that new cell lines be grown, mRNA harvested, and then the analysis compared. Technical replicates, on the other hand, analyze the same RNA twice. A line cutoff is then drawn just above where the replicates branch such that cells branching below the cutoff line are considered the same cell type. Another source of data for the database described above may be microRNA profiles of the single cell-derived and oligoclonal cell-derived cells generated according to the methods of the present invention. MicroRNAs (miRNA) are endogenous RNAs of ˜22 nucleotides that play important regulatory roles in animals & plants by targeting mRNAs for cleavage or translational repression. More than 700 miRNAs have been identified across species. Their expression levels vary among species and tissues. Low abundant miRNAs have been difficult to detect based on current technologies such as cloning, Northern hybridization, and the modified Invader® assay. In the present invention, an alternative approach using a new real-time quantitation method termed looped-primer RT-PCR was used for accurate and sensitive detection of miRNAs as well as other non-coding RNA (ncRNA) molecules present in human embryonic stem cells and in cell lines differentiated from human embryonic stem cells.

In another embodiment of the invention, gene expression analysis may be used to identify the developmental pathways and cell types for in vitro differentiated hES cells. Gene expression analysis of single cells or a small number of cells from human or nonhuman embryonic or fetal tissues provides another means to generate a database of unique gene expression profiles for distinct populations of cells at different stages of differentiation. Gene expression analysis on single cells isolated from specific tissues may be performed as previously described by Kurimoto et al., Nucleic Acids Research (2006) Vol. 34, No. 5, e42. Thus, cellular miRNA profiles on their own or in conjunction with gene expression profiles, immunocytochemistry, and proteomics provide molecular signatures that can be used to identify the tissue and developmental stage of differentiating cell lines. This technique illustrates that the database may be used to accurately identify cell types and distinguish them from other cell types.

The cells of the present invention are also useful in providing a subset of gene expression markers that are expressed at relatively high levels in some cell lines while not be expressed at all in other cell lines as opposed to genes expressed in all cell lines but at different levels of expression. This subset of “all-or none” markers can be easily identified by comparing the levels of expression as measured for instance through the use of oligonucleotide probes or other means know in the art, and comparing the level of a gene's expression in one line compared to all the other lines of the present invention. Those genes that are expressed at relatively high levels in a subset of lines, and not at all in other lines, are used to generate a short list of gene expression markers. When applied to the cells and gene expression data described herein, where negative expression on the Illumina bead arrays is <70 RFU and positive expression is >100 RFU.

The cells of the present invention may be used for drug screening to determine the toxic or beneficial effects of a test compound. For example toxicity with respect to endothelial cells or endothelial progenitors may be tested. As another example toxicity with respect to myoblast or myoblast progenitors may be tested. Thus a cell or a population of cells according to the invention can be contacted with a test compound and the effects of the test compound may be determined by comparing the effects on an identical cell or cell population that has not been contacted with the test compound. Suitable parameters for analysis include growth rate, doubling time, cell death and protein and mRNA expression in the two cells or two cell populations.

### Neural Differentiation Medium 2

The cell line to be tested is plated in six well plates at two different densities 5×10⁵ cells/well. The cells are grown under standard growth conditions until they reach confluence. The media is then replaced with 50% DMEM 50% F12 media supplemented with N2 containing and MEM-NEAA, 2 mg/ml heparin, 1 mM cAMP, 200 ng/ml ascorbic acid, 50 ng/ml IGF-1, 10 ng/ml GDNF, 10 ng/ml BDNF).

### Tissue Engineered Constructs

In certain embodiments, cells of the present invention are employed in therapeutic applications to repair, replace, or enhance tissue function in a subject (e.g, a mammal, e.g., a human patient). A number of therapies that employ cells incorporated in engineered matrices have been described, a few of which are summarized below. The cells of the present invention may be embedded in such matrices to provide form and function as is well-known in the art.

In certain embodiments, synthetic matrices or biological resorbable immobilization vehicles (sometimes referred to as “scaffolds”) may be impregnated with cells of the present invention. A variety of synthetic carrier matrices have been used to date and include: three-dimensional collagen gels (U.S. Pat. No. 4,846,835; Nishimoto (1990) Med. J. Kinki University 15; 75-86; Nixon et al. (1993) Am. J. Vet. Res. 54:349-356; Wakitani et al. (1989) J. Bone Joint Surg. 71B:74-80; Yasui (1989) J. Jpn. Ortho. Assoc. 63:529-538); reconstituted fibrin-thrombin gels (U.S. Pat. Nos. 4,642,120; 5,053,050 and 4,904,259); synthetic polymer matrices containing polyanhydride, polyorthoester, polyglycolic acid and copolymers thereof (U.S. Pat. No. 5,041,138); and hyaluronic acid-based polymers (Robinson et al. (1990) Calcif. Tissue Int. 46:246-253).

For example, the cells of the present invention may be employed in tissue reconstruction as described in Methods of Tissue Engineering (2002), edited by Anthony Atala and Robert P. Lanza and published by Academic Press (London), incorporated by reference herein for its description of tissue reconstruction (see, e.g, pages 1027 to 1039). As described therein, cells may be placed into a molded structure (e.g., by injection molding) and transplanted into an animal, Over time, tissue produced by the cells of the present invention will replace the molded structure, thereby producing a formed structure (i.e., in the shape of the initial molded structure). Exemplary mold materials for the molded structure include hydrogels (e.g., alginate, agarose, polaxomers (Pluronics)) and natural materials (e.g., type I collagen, denatured type I collagen (gelatin), type II collagen, hyaluronic acid, polymers of type I collagen and hyaluronic acid such as HyStem [see, e.g., U.S. Pat. No. 7,981,871 titled “Modified Macromolecules and Associated Methods of Synthesis and Use” and U.S. Pat. No. 7,928,069 titled “CROSSLINKED COMPOUNDS AND METHODS OF MAKING AND USING THEREOF”, both of which are incorporated by reference herein in their entirety] and fibrin).

In certain embodiments, cells of the present invention may be cultured in vitro to form a synthetic tissue-like material. The resulting tissue may be implanted subsequently into a subject at the site of the defect. This type of approach has the advantage that the development of the synthetic tissue may be monitored prior to implantation. In addition, the resulting tissue may be characterized biochemically and morphologically prior to implantation. Numerous different procedures have been developed for growing synthetic tissue in vitro, including growing cells in an anchorage-dependent or an anchorage-independent manner.

In certain embodiments, the vascular endothelial cells of the present invention may be attached to a tissue engineered substrate as described herein to form tubular structures to function as tissue engineered arteries, veins, and lymphatic vessels.

In certain embodiments, the tissue engineered arteries, veins, and lymphatic vessels with endothelial cells on the luminal side may be combined with smooth muscle progenitors, smooth muscle cells, pericytes, or pericyte progenitors.

### Direct Injection of Cells to Impart Tissue Regeneration

Direct injection of cells, such as the cell lines of the present invention are also of therapeutic utility. Doses and formulation will vary depending on the route of administration, tissue type, and nature of the pathology to be treated as is known in the art, but in the case of humans and most veterinary animals species, the dosage will be between 10²-10⁹ cells and the formulation can be, by way of nonlimiting example, a cell suspension in isosmotic buffer, a hydrogel with cell adhesion sites including but not limited to polymers of hyaluronic acid and collagen (see, e.g., U.S. Pat. No. 7,981,871 titled “Modified Macromolescules and Associated Methods of Synthesis and Use” and U.S. Pat. No. 7,928,069 titled “CROSSLINKED COMPOUNDS AND METHODS OF MAKING AND USING THEREOF”, both of which are incorporated by reference herein in their entirety), or a monolayer of cells attached to an layer of extracellular matrix such as contracted gelatin wherein said monolayer is attached to a surface delivered by a catheter such as a catheter delivering vascular endothelial cells to diseased arteries. Cellular compositions of the present invention may further comprise an acceptable carrier, such as a hydrophilic, e.g., pharmaceutically acceptable, carrier.

### Systems and Kits

Also provided by the subject invention are systems and kits that include the cells of the invention for use in various applications, as described herein. The systems and kits may further include reagents and materials for the propagation and use of the cells for research and/or therapeutic applications as described herein.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### hES Cell-Derived Monoclonal Embryonic Progenitor Cell Lines Generated in the Presence of SB431542

### Differentiation of hES Cells

The human embryonic stem cell line ESI-017 (Crook et al, (2007) The Generation of Six Clinical-Grade Human Embryonic Stem Cell Lines, Cell Stem Cell 1: 490-494) was maintained on mouse embryonic fibroblasts (MEFs) in hESC medium consisting of Knockout DMEM (Invitrogen) supplemented with 20% knockout serum replacement (Invitrogen), 1× non-essential amino acids (Invitrogen), 1× Glutamax-1 (Invitrogen) and used at passage 33. The cells were differentiated as described (Dayton, J. et al, (2010) Expansion and maintenance of human embryonic stem cell-derived endothelial cells by TGFbeta inhibition is Id1 dependent. Nat Biotechnol. 28:161-166); see U.S. Provisional Application No. 61/290,667, filed on Dec. 29, 2009 and titled “Methods For Developing Endothelial Cells From Pluripotent Cells And Endothelial Cells Derived”, incorporated herein by reference) with the modifications described herein and summarized in FIG. 1. The hES cells were dispersed into single cells with Accutase. Embryoid bodies were prepared by centrifugation of the cells in Aggrewell plates (Stem Cell Technologies) (4 wells, 3 million cells/well). There are about 1,200 microwells/well of an Aggrewell plate therefore each embryoid body initially contained approximately 2,500 cells and were cultured for 24 hours in basal differentiation medium (consisting of Stemline II (Sigma Cat# S0192) with no supplements. On the morning after generation of embryoid bodies (day 0), medium was supplemented with 20 ng/ml BMP4 (R&D Systems) (removed at day 7); On day 1, the embryoid bodies were removed from the Aggrewell plates and transferred to a 6-well Ultra Low Binding plate (Corning) with basal differentiation medium supplemented with 20 ng/ml BMP4 and 10 ng/ml Activin A (R&D Systems) (Activin A removed at day 4). On day 2, medium was replaced with basal differentiation medium supplemented with 20 ng/ml BMP4, 10 ng/ml Activin A (Activin A removed at day 4) and 8 ng/ml bFGF (Millipore) (removed at day 7); on day 4, embryoid bodies were transferred to adherent conditions on 1:30 diluted Matrigel-coated plates (Corning) and medium was supplemented with 20 ng/ml BMP4, 8 ng/ml bFGF, and 25 ng/ml VEGF-A (Peprotech) (removed at day 7); on day 6, SB431542 (Cayman) was added at a concentration of 10 uM.

### Generation of Candidate Cultures

Prior to clonal isolation, cells were propagated in the same media in which they will subsequently be cloned. These preliminary cultures of heterogeneous cells are designated “candidate cultures”. To prepare the candidate cultures, the above-mentioned day 7 cultures were dissociated with 0.25% Trypsin (Invitrogen) for 10 minutes at 37 C followed by trituration (i.e. repeated pipetting up and down) with a Pipetman to generate a single cell preparation. The cell suspension was diluted with 3 nil of DMEM+5% FBS (4 ml total) and 1 ml of cell suspension was transferred into each of 4 wells of a 6 well cell culture plate coated with Matrigel containing 4 ml of 4 different media: 1) Promocell endothelial MV2 media with supplements at concentrations normally recommended by the manufacturer and sold as a complete kit (Cat# C-22022) or as cell basal medium (Cat# C-22221) and growth supplement (Cat# C-39221) until the cells reached confluence; 2) Promocell smooth muscle cell basal medium (Cat# C-22062B) and growth supplement (Cat# C-39267); 3) Promocell skeletal muscle basal medium (Cat# C-22060B) and growth supplement (Cat# C-39365); and 4) DMEM media supplemented with 10% FCS. All media used were supplemented with the TGFβ inhibitor SB431542. The media were changed 24 hours after initial plating and twice weekly thereafter. On confluence of cells in the 6-well plate, cells were trypsinized and replated in progressively larger culture flasks being: 125 Matrigel coated flask, T75 Matrigel coated flask, then a T225 Matrigel coated flask each in their respective 4 different media. Cells in the final T225 flask were trypsinized, cells were thoroughly dispersed to a single cell suspension by trituration in 1.0 ml of its growth medium, counted, diluted so that the final cell concentration is approximately 10,000 cells/ml, single cell dispersion confirmed by light microscopy, then plated at clonal dilution (500 and 2,000 cells) in 15 cm Matrigel coated dishes in the respective four media. Remaining cells were cryopreserved (typically 2×10⁶ to 5×10⁶ cells/vial) as candidate cultures for future new rounds of cloning.

### Clonal Dilution of hES Progenitor Cells

Cloning dishes were prepared by adding 50 ml of the above-referenced respective media into Matrigel-coated 15 cm culture dishes. To each dish, cells from the candidate cultures propagated in each of the above-referenced media were added such that 500 cells (50 ul of 10,000 cells/ml diluted cells to a dish containing medium), a second dish was similarly prepared at 1,500 cells/dish, and a third at 5,000 cells/dish. For the 4 media types, a total of 12 dishes were prepared. Cells were distributed evenly in the 15 cm dish by a sliding the dish alternately, in a side to side (left to right) motion followed by a forward and back motion repeatedly for about 1 minute in the incubator. Dishes were incubated in a CO2 incubator preferably with 5% oxygen undisturbed for 14 days.

### Selection of Colonies and Propagating Clonal Lines

Dishes are visually inspected for colonies picked with 6, 8, or 10 mm sterile cloning cylinders using 25 ul trypsin for a 6 mm cylinder, 50 ul trypsin for an 8 mm cylinder, and 100 ul trypsin for a 10 mm cylinder. Cells are replated into Matrigel-coated 24 well plates containing 1 ml of respective medium per well. Cells in the 24 well plate once confluent were trypsinized and transferred to Matrigel-coated 6 well plate, then progressively Matrigel-coated T25, 175, and T225 flasks and cryopreserved. The cell lines of the present invention designated RP1-DM10-1, RP1-DM10-13, RP1-DM10-18, and RP1-DM10-22 were isolated in DMEM media supplemented with 10% FCS. The cell lines of the present invention designated RP1-MV2-1, RP1-MV2-2, RP1-MV2-3, RP1-MV2-5, RP1-MV2-6, RP1-MV2-7, RP1-MV2-8, RP1-MV2-9, RP1-MV2-10, RP1-MV2-11, RP1-MV2-12, RP1-MV2-13, RP1-MV2-14, RP1-MV2-15, RP1-MV2-16, RP1-MV2-18, and RP1-MV2-23 were isolated in Promocell endothelial MV2 media with supplements at concentrations normally recommended by the manufacturer and sold as a complete kit (Cat# C-22022) or as cell basal medium (Cat# C-22221) and growth supplement (Cat# C-39221). The cell lines RP1-SM2-2, RP1-SM2-4, RP1-SM2-10, RP1-SM2-11, RP1-SM2-12, RP1-SM2-13, RP1-SM2-15, RP1-SM2-16, RP1-SM2-21, RP1-SM2-22, and RP1-SM2-23 were isolated in Promocell smooth muscle cell basal medium (Cat# C-22062B) and growth supplement (Cat# C-39267). The cell lines RP1-SKEL-1, RP1-SKEL-3, RP1-SKEL-5, RP1-SKEL-6, RP1-SKEL-8, RP1-SKEL-11, RP1-SKEL-15, RP1-SKEL-16, RP1-SKEL-19, RP1-SKEL-20, RP1-SKEL-21, RP1-SKEL-22, and RP1-SKEL-23 were isolated in Promocell skeletal muscle basal medium (Cat# C-22060B) and growth supplement (Cat# C-39365). The cell lines RP1-DM10-1, RP1-DM10-13, RP1-DM10-18, RP1-DM10-19, and RP1-DM10-22 were isolated in DMEM medium supplemented with 10% FCS.

Preparation of Cells for RNA isolation for Gene Expression analysis

The cell lines of the present invention at passage 6 (corresponding to approximately 26 doublings of clonal expansion) were grown in Matrigel-coated 6 well plates to confluence. Media was replaced with 10% low supplement medium (For each media type, dilute complete medium 1:10 with basal (no supplement) medium. The cells were allowed to become quiescent in low serum media for 5 days with 1 change of medium after 3 days. Cells were lysed with 350 ul/well of Qiagen RLT buffer and lysate transferred to 1.5 ml RNAse free Eppendorf tubes and stored at −80 C until ready to send for gene expression analysis. RNA was then analyzed on Illumina gene expression microarrays. The rank normalized data was sorted to display gene RFU values for each cell line in order of the highest variation from the mean RFU (shown as a “hot pop” value ((RFU for that gene in that cell line-average RFU value for that gene in all cell lines)/average RFU value for that gene in all cell lines) for that gene in all cell lines analyzed.

The clonal cell lines designated RP1-MV2-6 (at passage 6), RP1-MV2-16 (at passage 6), and RP1-MV2-18 (at passage 6) displayed high levels of the endothelial markers including but not limited to: VWF, CDH5, PECAM, ICAM2, and ESAM The cell lines RP1-MV2-6 and RP 1-MV2-18 expressed no HOXA, HOXC, or HOXD gene expression, and the most distal HOX expression being HOXB8 and did express HOXB4. The cell line RP 1-MV2-16 showed the most distal HOX gene expression of HOXA10, HOXB8 (with low or undetectable HOXB4), and did not express HOXC or HOXD genes. Therefore the methods used to derived these clonally-purified hES-derived endothelial cell lines RP1-MV2-6, RP1-MV2-16, and RP1-MV2-18 at comparable purity of the embryonic endothelial cell lines of the present invention (i.e. 98.3-100% pure Pecam-I positive cells), and cells with comparably long telomere length and proliferative lifespan are useful in research and regenerative therapies such as for increasing blood flow in aged, ischemic, or otherwise diseased tissues of humans or animals. They may be produced from clinical grade hES cells or reprogrammed iPS cells wherein the hES or iPS cells are or are not genetically modified as described herein. Said markers may vary as is known in the art with the type of microarray or alternative means of measuring mRNA levels in said cells and may vary with passage of the culture in vitro. The values shown in Table VI are indicative of said clonal endothelial cell lines at approximately 26 doublings of clonal expansion.

The clonal cell lines RP1-SKEL-8 and RP1-SM2-12 (each as passage 6 and again representing approximately 26 doublings of clonal expansion) expressed markers of skeletal muscle myoblasts. In particular, the cell line RP1-SKEL-8 showed high levels of expression of MYH3, MYBPH, ACTA1, MYL1, and the most distal HOX gene expression of HOXA10 (Illumina probe ID ILMN_{—}12295), HOXB8, and HOXC8, and the cell line RP1-SM2-12 expressed high levels of expression of MYH3, MYBPH, ACTA1, MYH7, MYL1, and the most distal HOX gene expression of HOXA5 (but no HOXA10 (Illumina probe ID ILMN_{—}12295)), HOXB8 and HOXC8. Therefore the methods used to derived these clonally-purified hES-derived myoblast cell lines and lines, with comparable purity, and cells with comparably long telomere length and proliferative lifespan are usefull in research and regenerative therapies such as for regenerating skeletal muscle damaged from trauma, infection, aging, or inherited diseases such as muscular dystrophy. Said myoblasts may or may not be genetically modified as described herein such as by the knockout or otherwise reducing the expression of myostatin (MSTN).

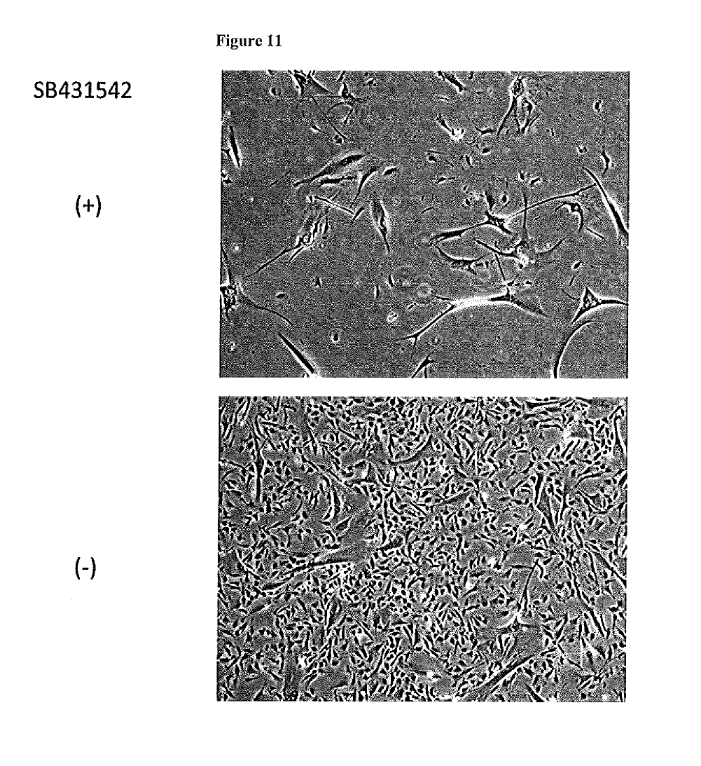

Removal of the TGFbeta inhibitor (SB431542) and culture on a gelatin substrate instead of Matrigel, leads to increased proliferation (FIG. 11).

### Example 2

### Initial Characterization of Endothelial Monoclonal Embryonic Progenitor Cell Lines Generated in the Presence of SB431542 Compared to Heterogeneous Cultures of hES-Derived Endothelium

Heterogeneous cultures of endothelial cells (i.e. cultures that were not clonally purified) were generated from ESI-51 cells according to Daylon, J, et al, (2010) Expansion and maintenance of human embryonic stem cell-derived endothelial cells by TGFbeta inhibition is Id1 dependent. Nat Biotechnol. 28:161-166 however some modifications were included in the protocol. Briefly, ESI-51 cells were plated into AggreWell plates (StemCell Technologies) at a density of 2.7×10⁶ cells per well in EGM2 media (Lonza) containing exclusively the following kit components: 2% fetal bovine serum, heparin, ascorbic acid, gentamycin and hydrocortisone. ESI-51 cells were cultured 4 days in the AggreWell plates allowing the formation of embryoid bodies (EBs) by sequential addition of BMP4, activin A, and basic FGF. At day 4 the EBs were collected and allowed to sediment by gravity 45 minutes. Upon removal of the conditioned media, the EBs were resuspended in fresh EGM2 media (including the kit components described above), containing BMP4, basic FGF and VEGF-2. At this point the EBs were transferred from suspension to adherent condition: The EBs were plated in T225 flasks previously coated with matrigel and they were allowed to attach and expand for 3 days. At day 7 of culture, the media was replaced with fresh EGM2 media (including the kit components described before), containing basic FGF, VEGF-2 and the TGF-β inhibitor SB431542 (Sigma Aldrich). At this time the cells experienced a rapid growth. The cells were always split before reaching confluence to avoid a reduction in cell growth upon cell to cell contact. Samples for microarray and FACS analysis were collected at different time points during the differentiation process. The endothelial cells derived following this procedure reached a peak of endothelial marker expression between days 20 and 28, a decrease in some endothelial marker expression was observed after the mentioned peak.

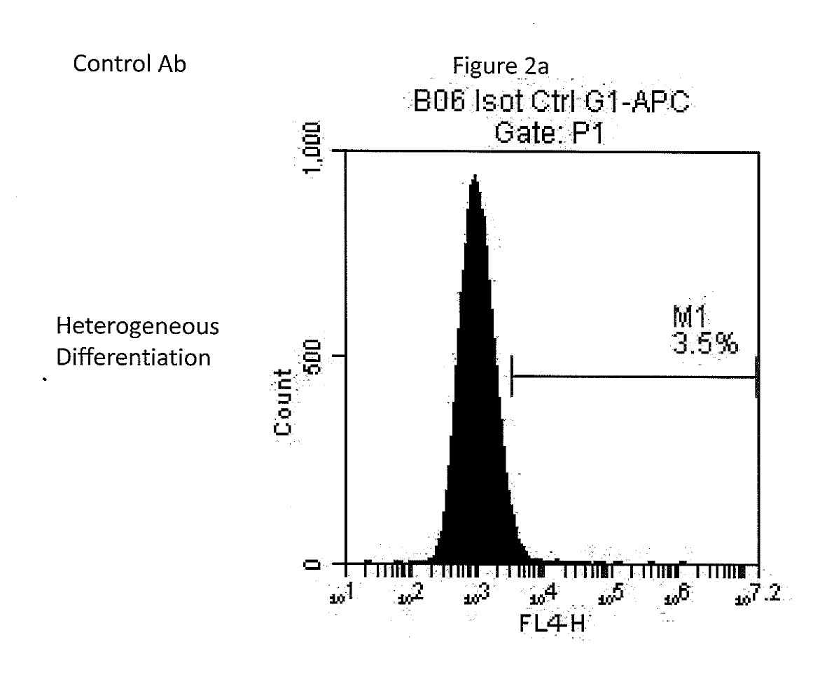

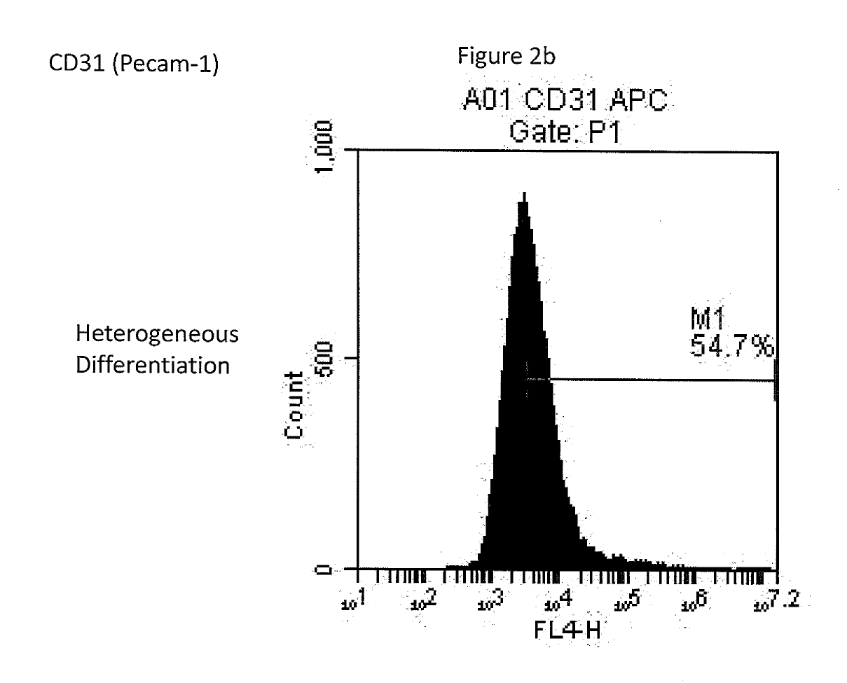

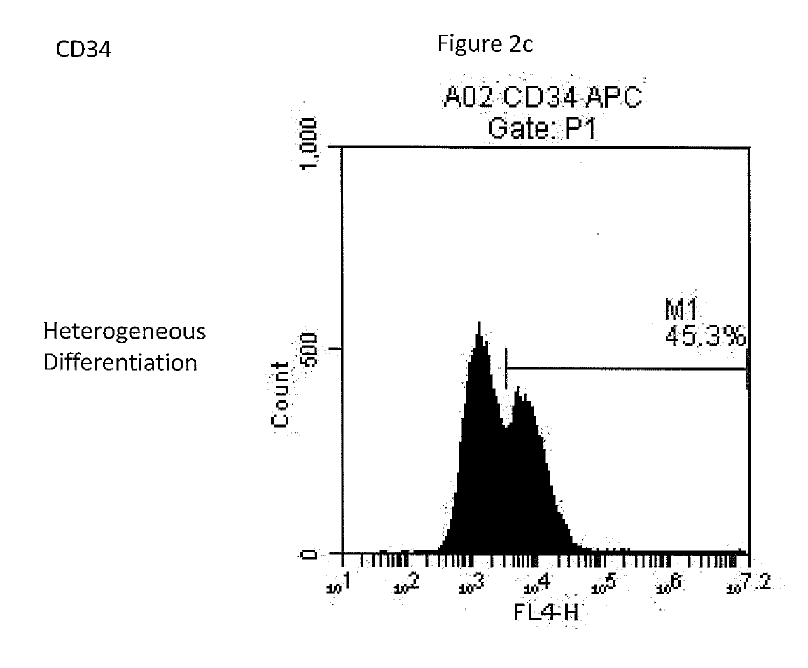

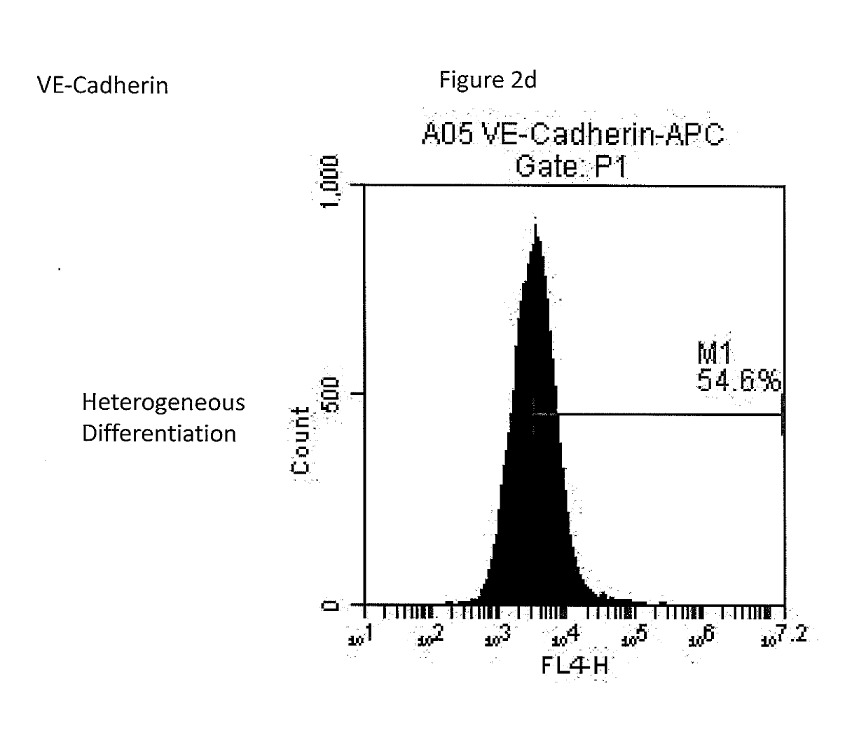

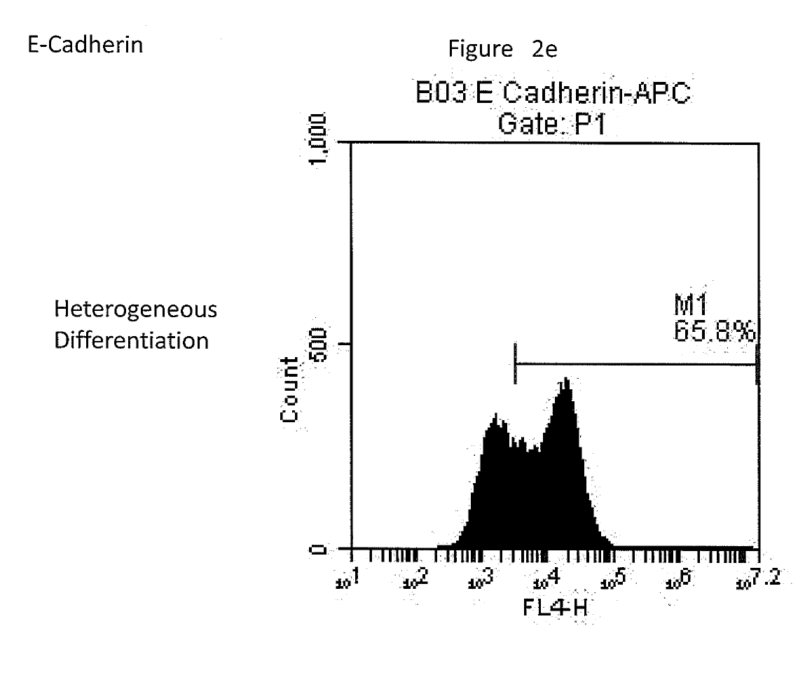

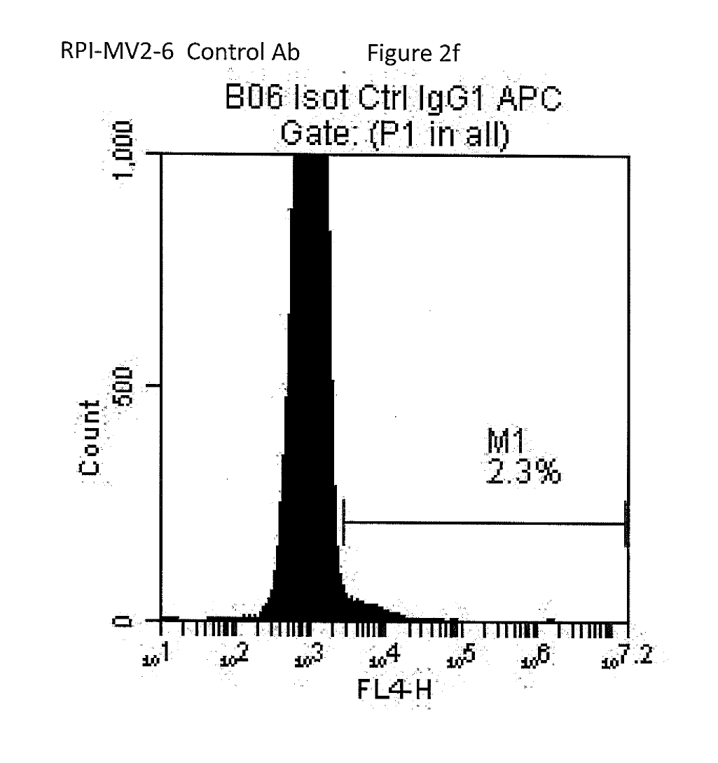

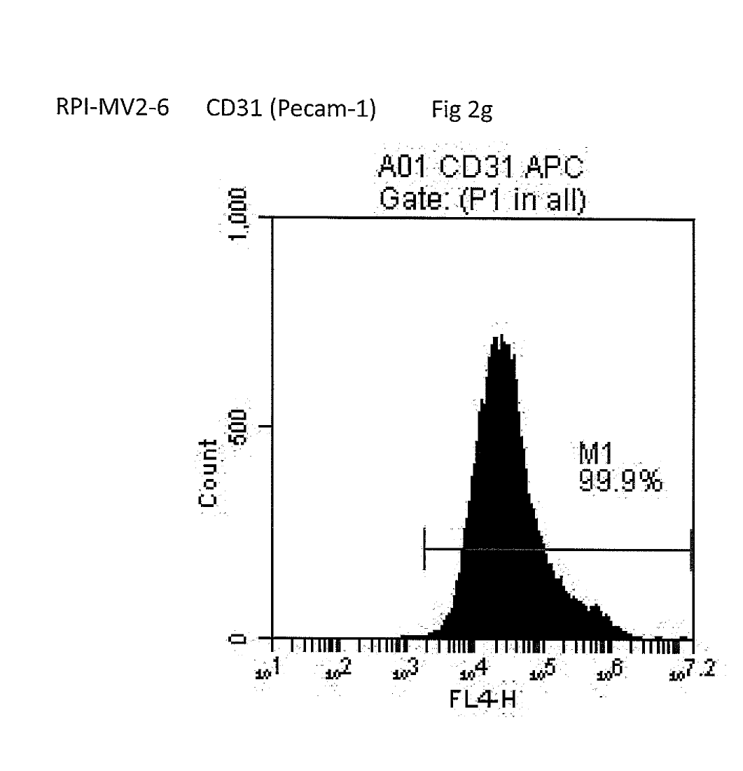

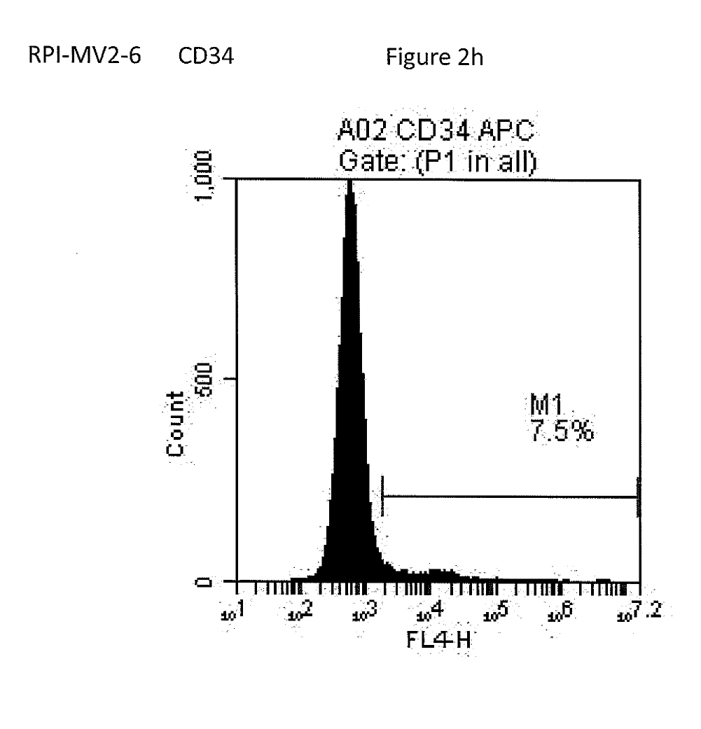

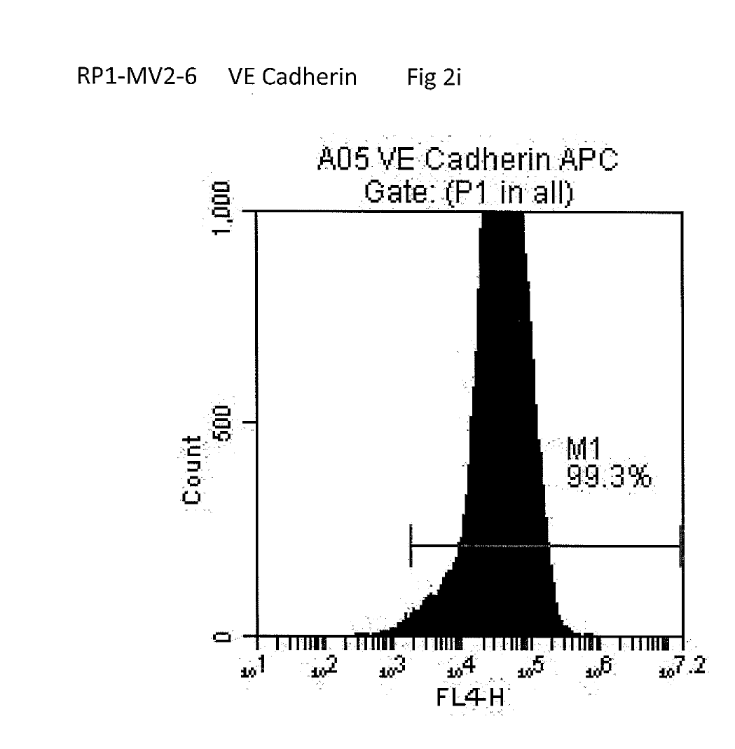

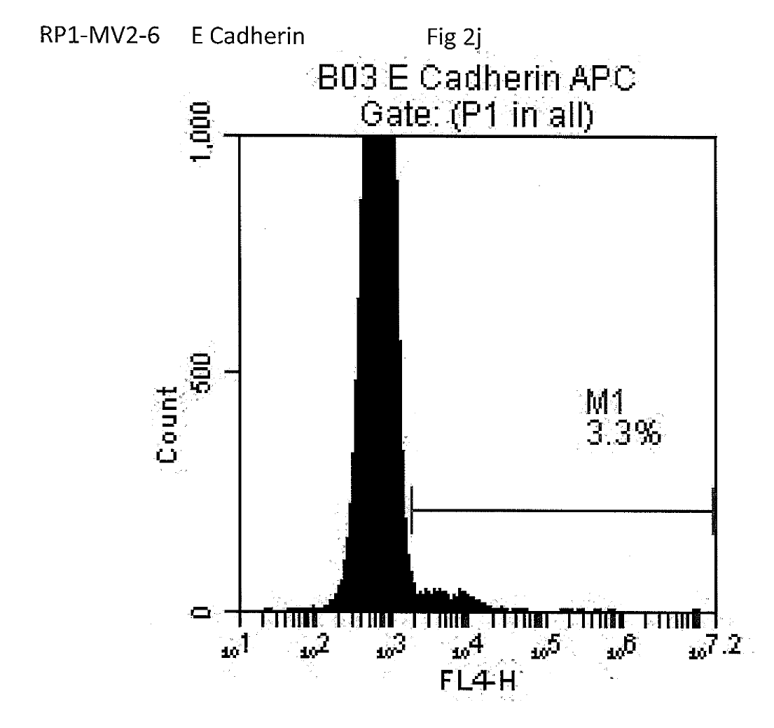

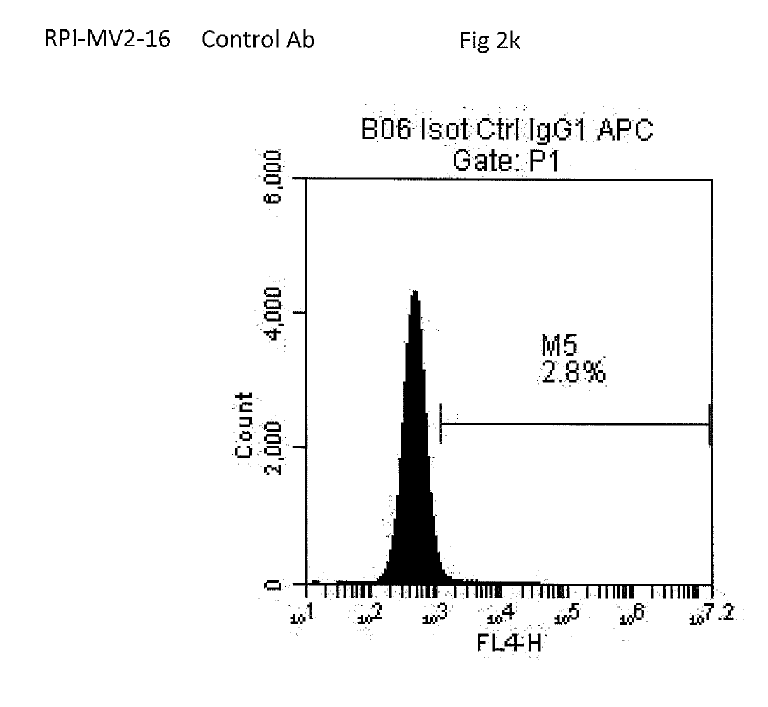

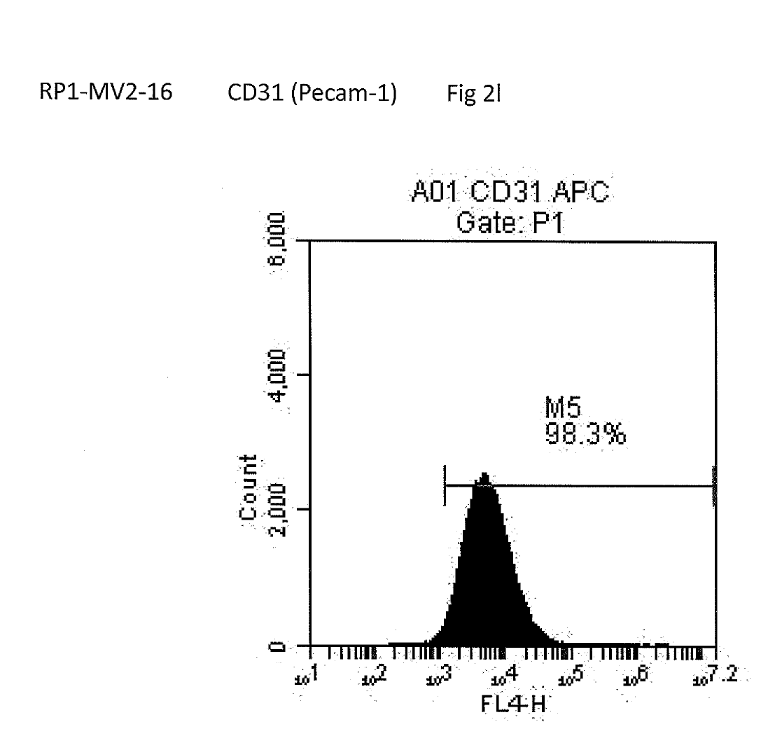

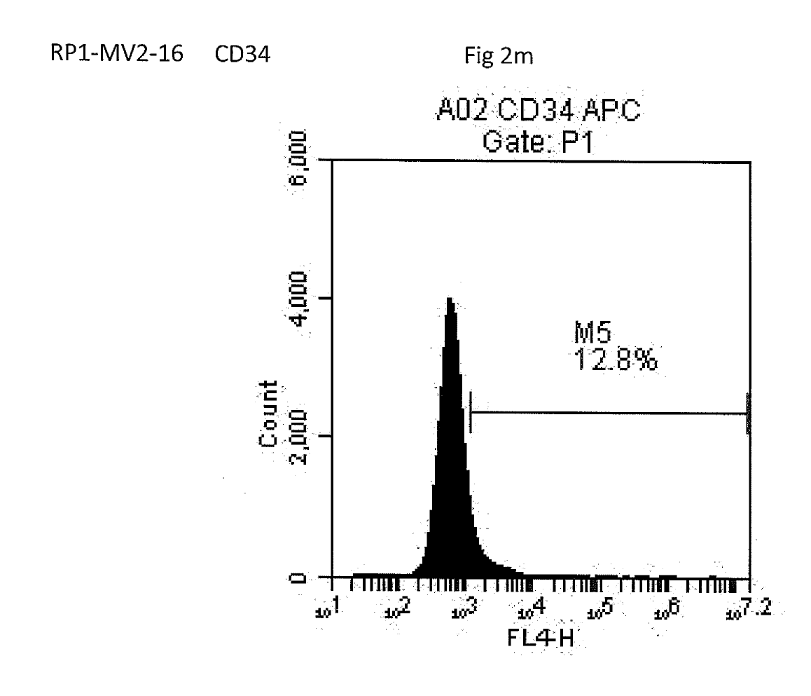

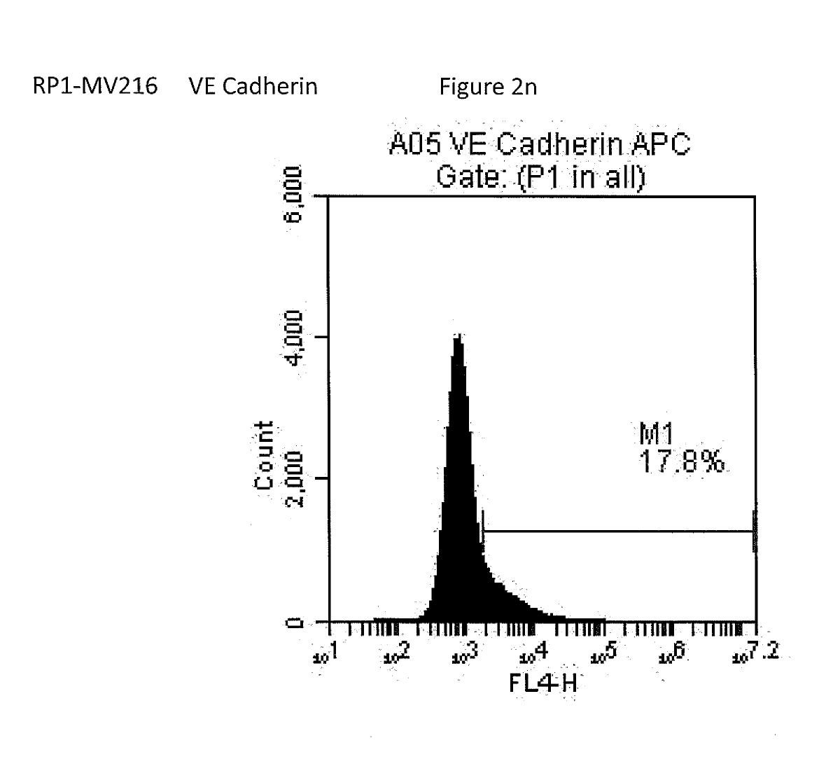

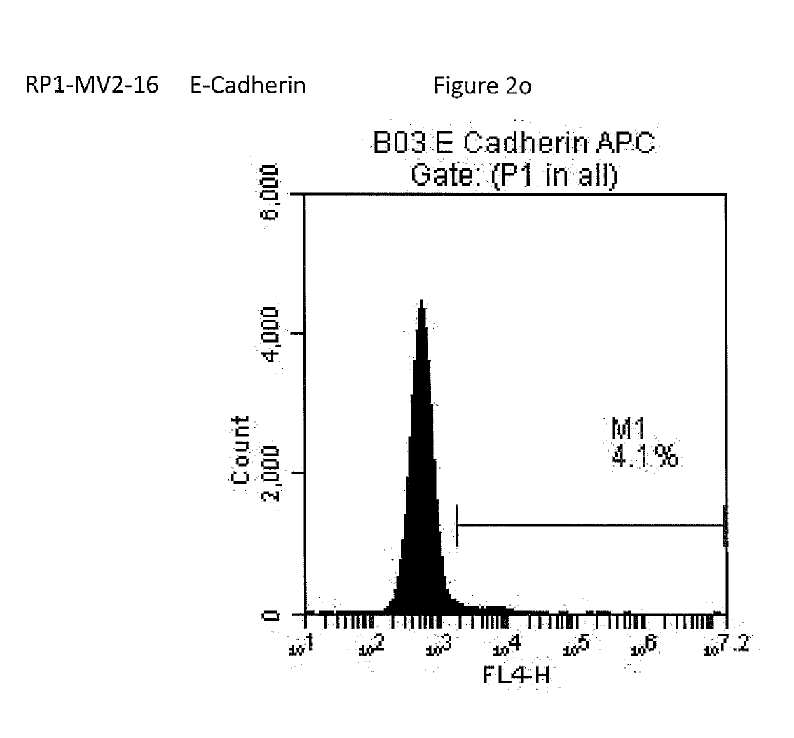

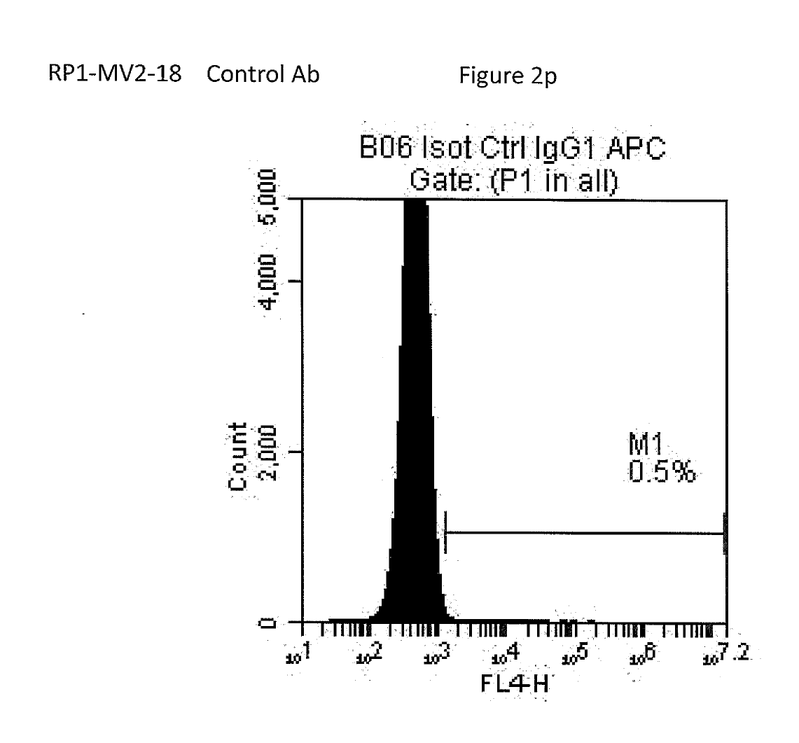

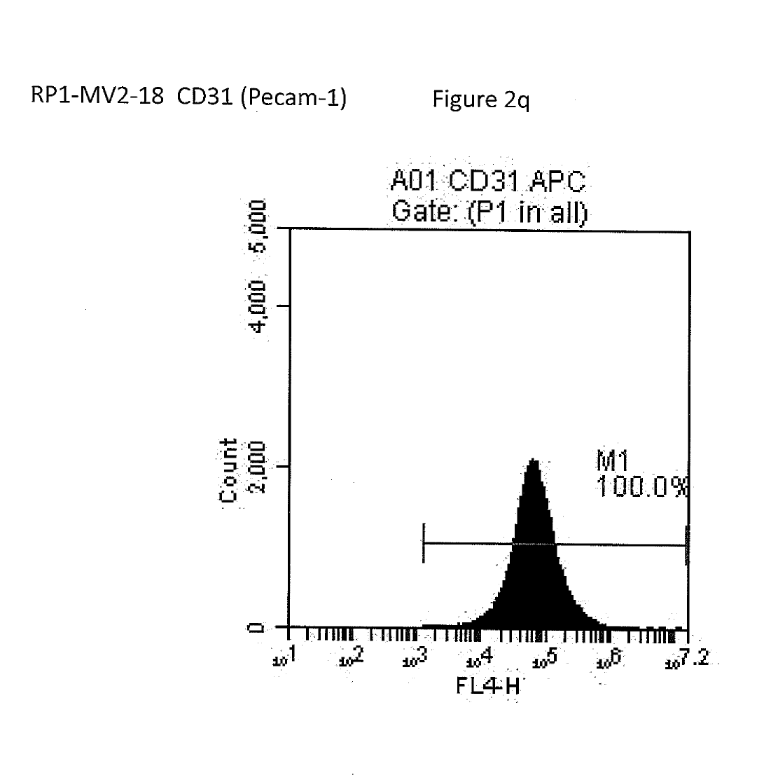

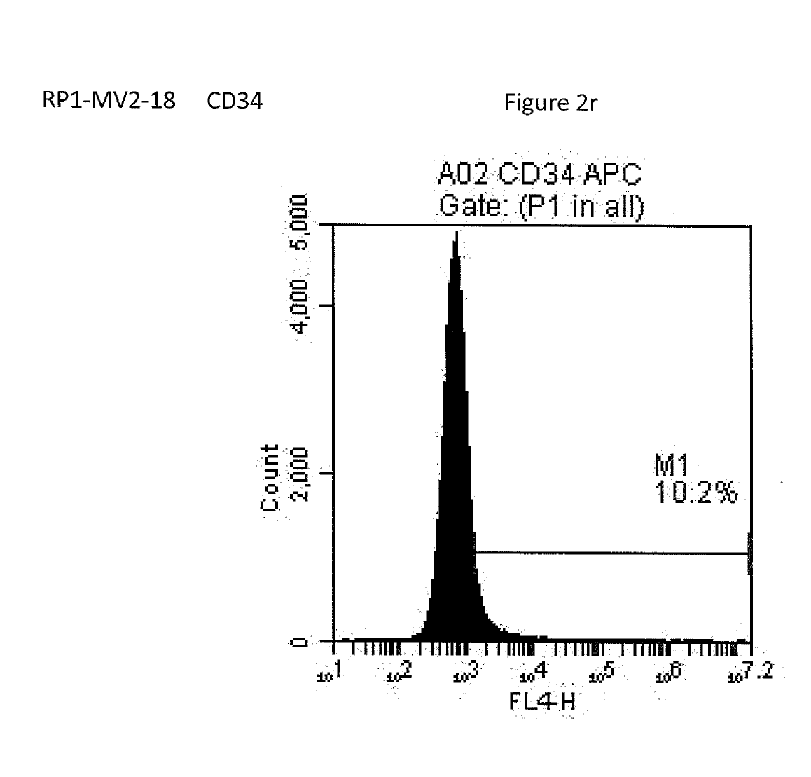

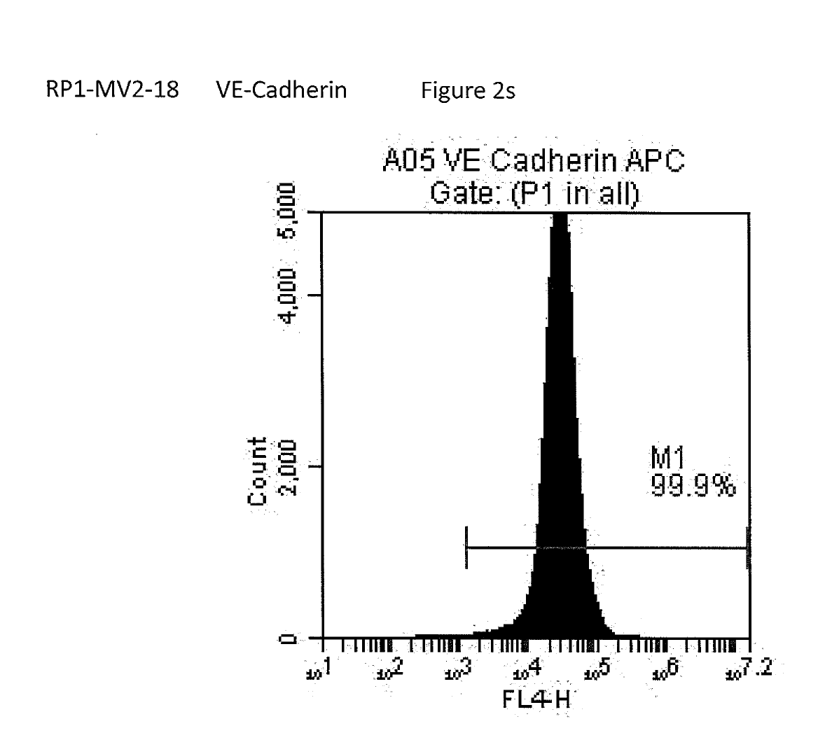

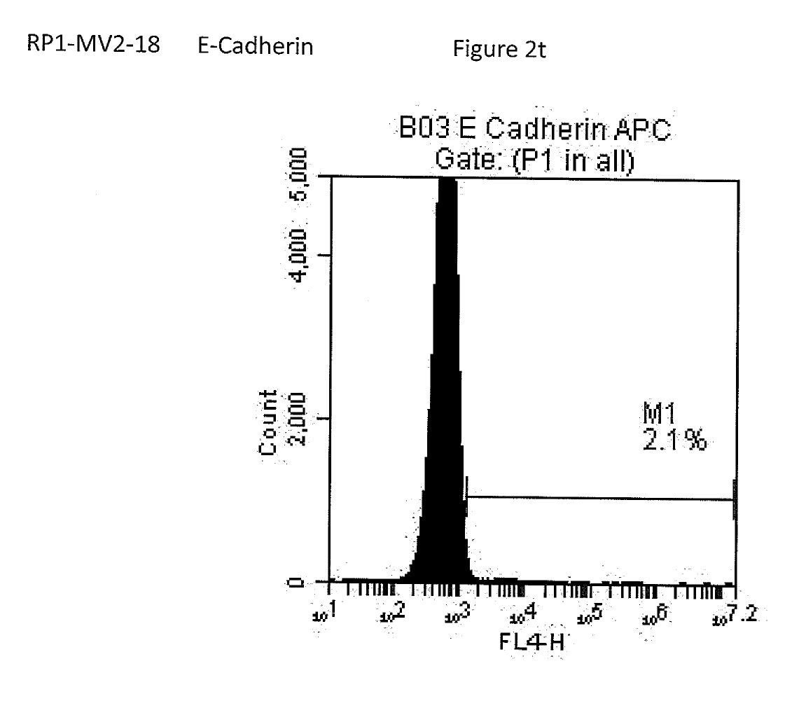

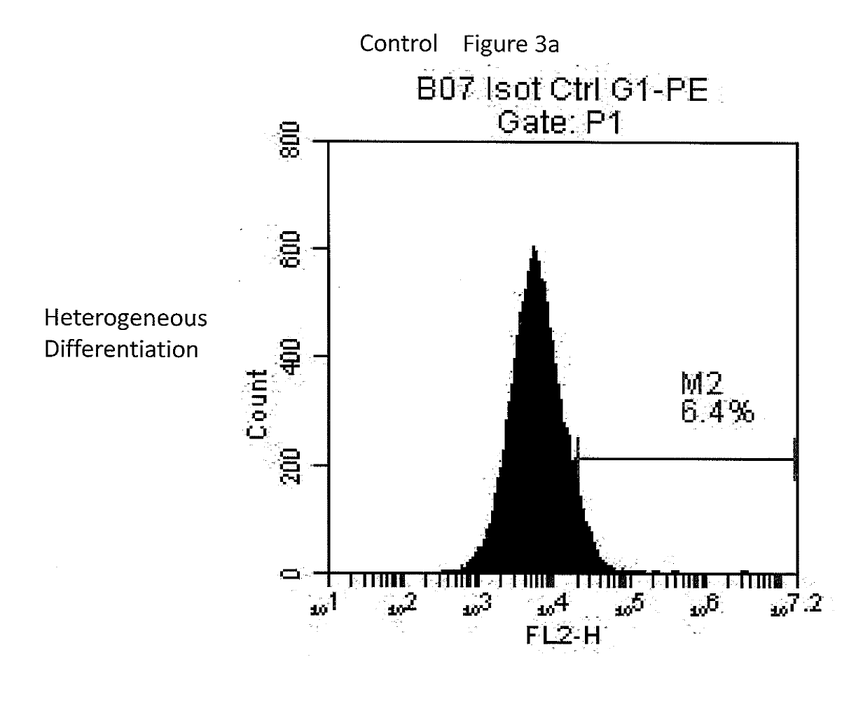

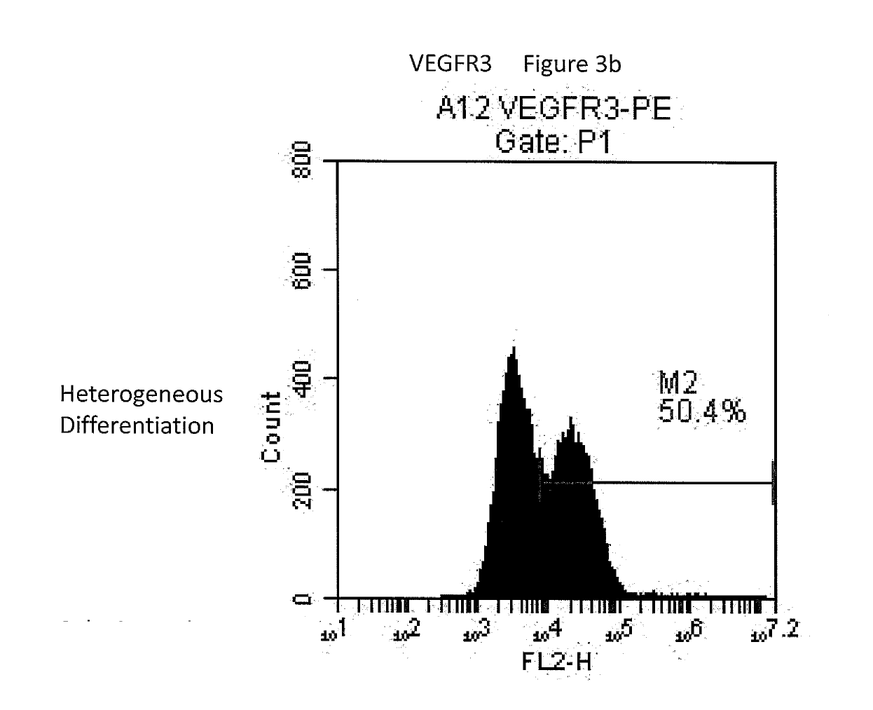

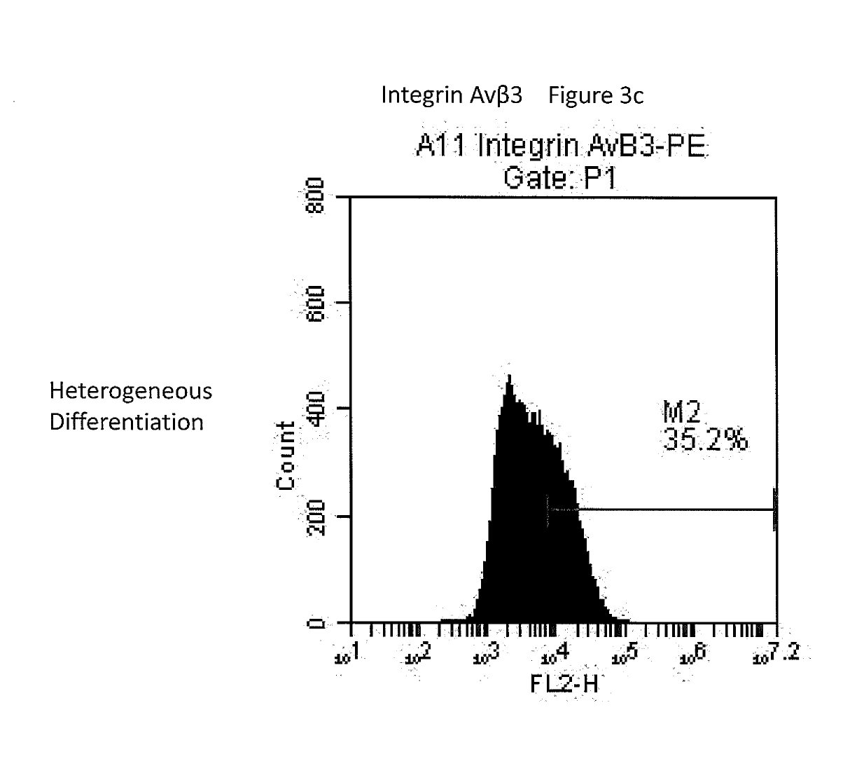

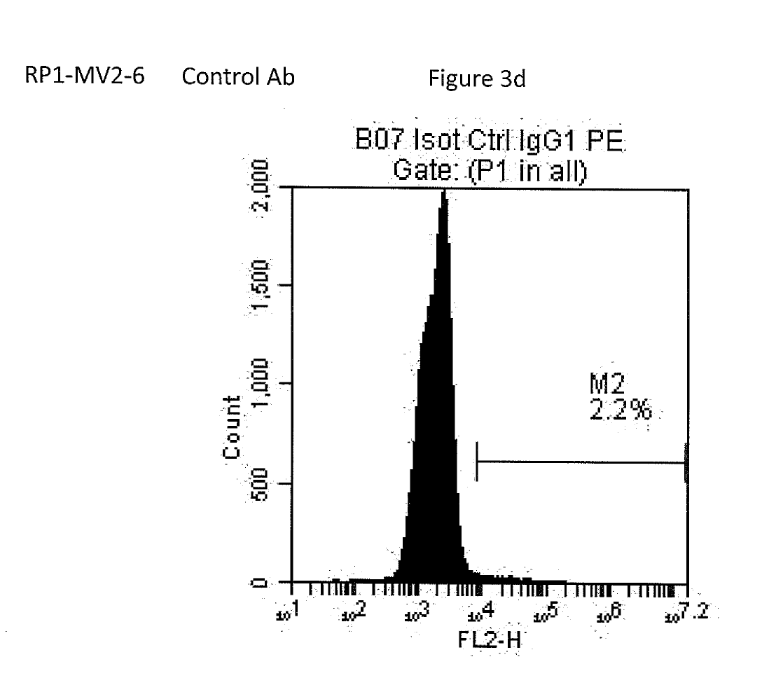

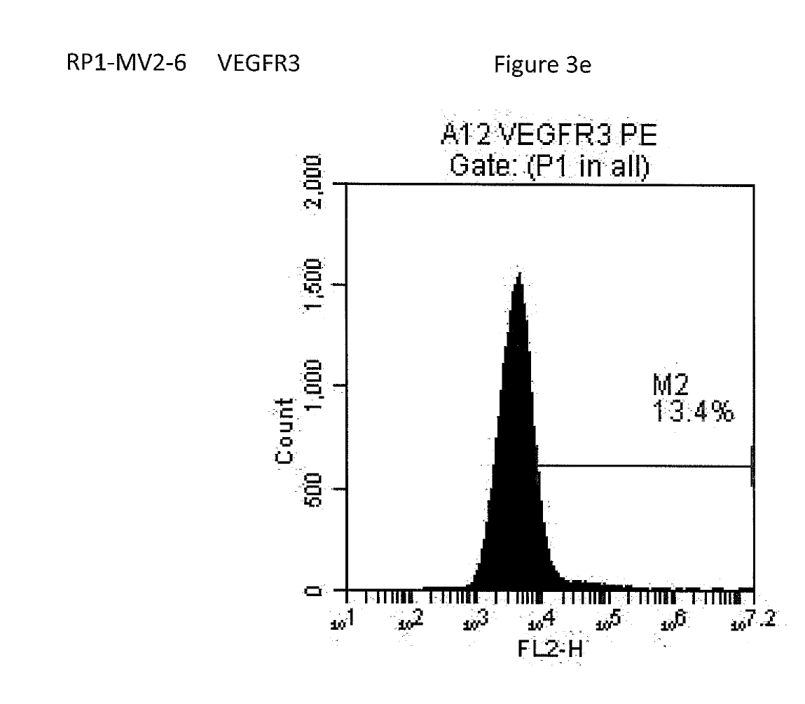

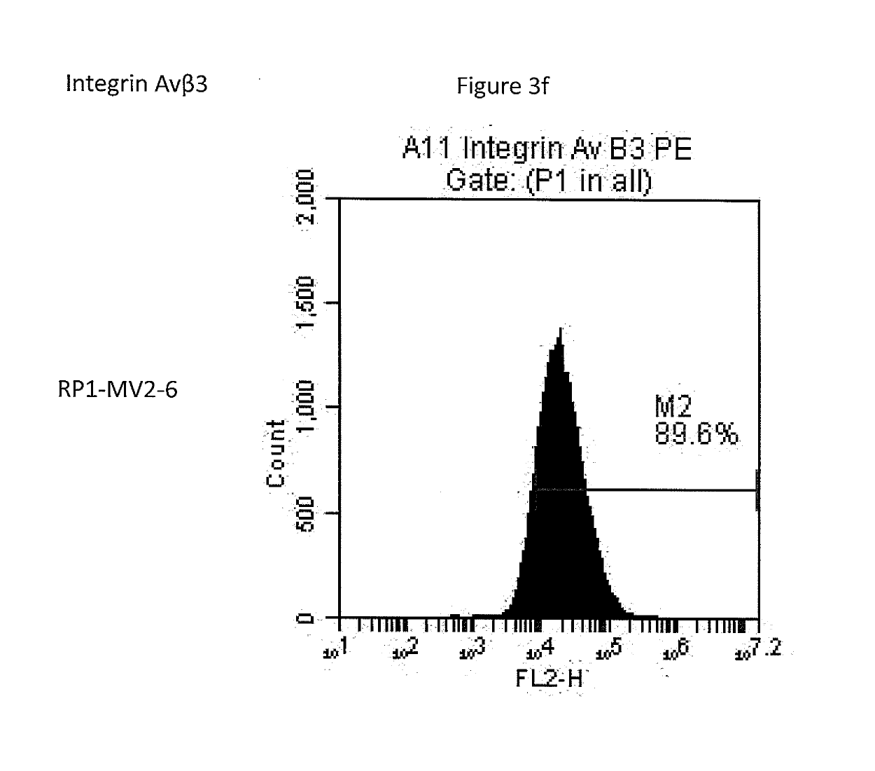

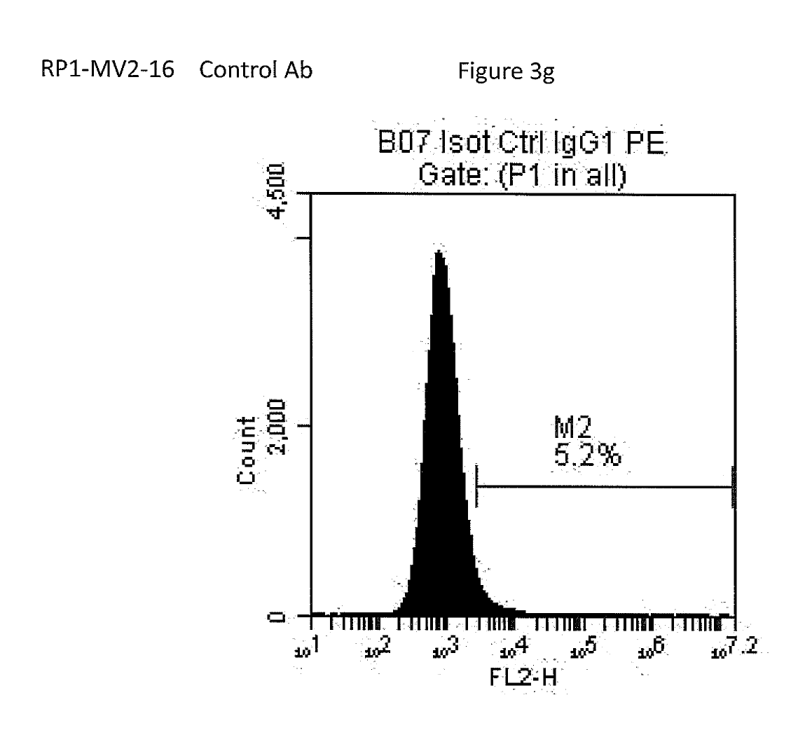

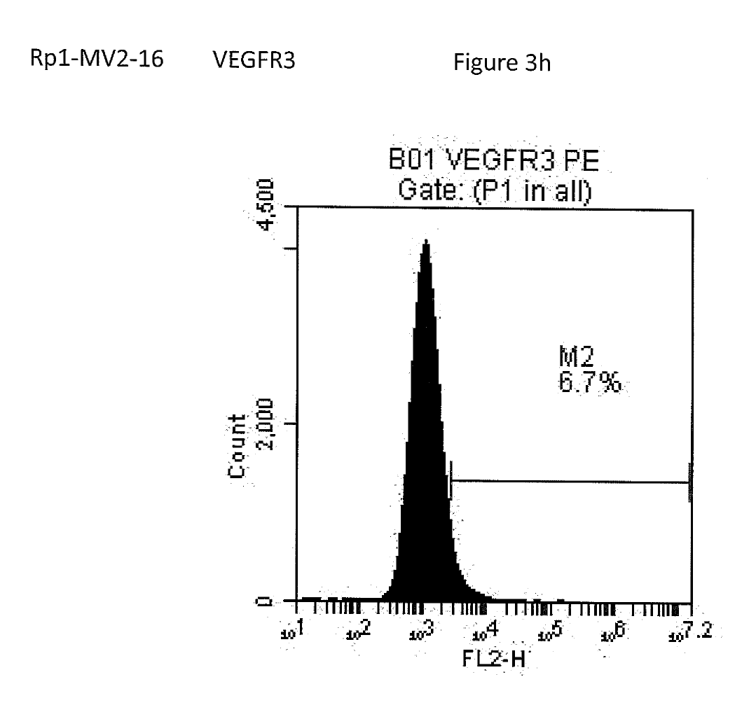

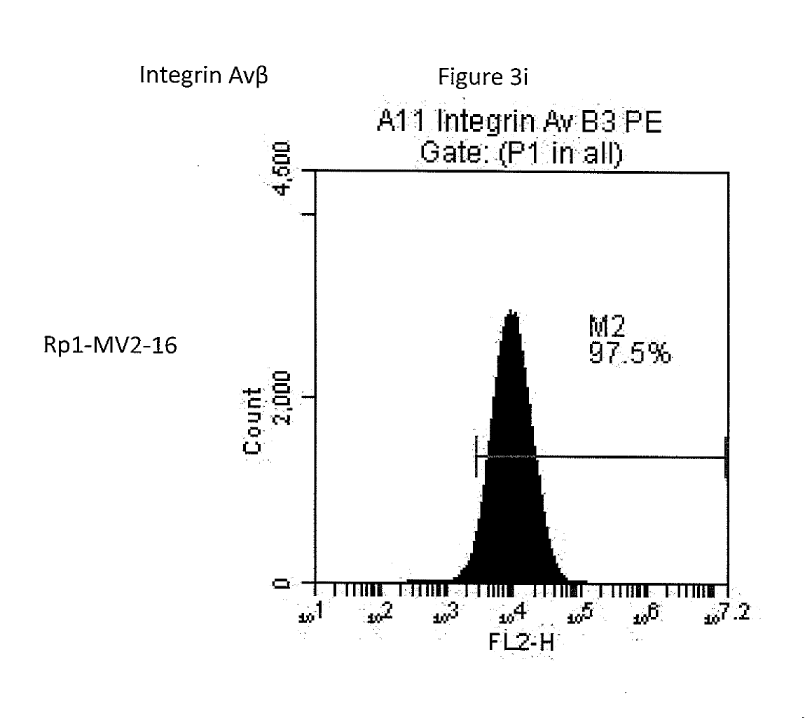

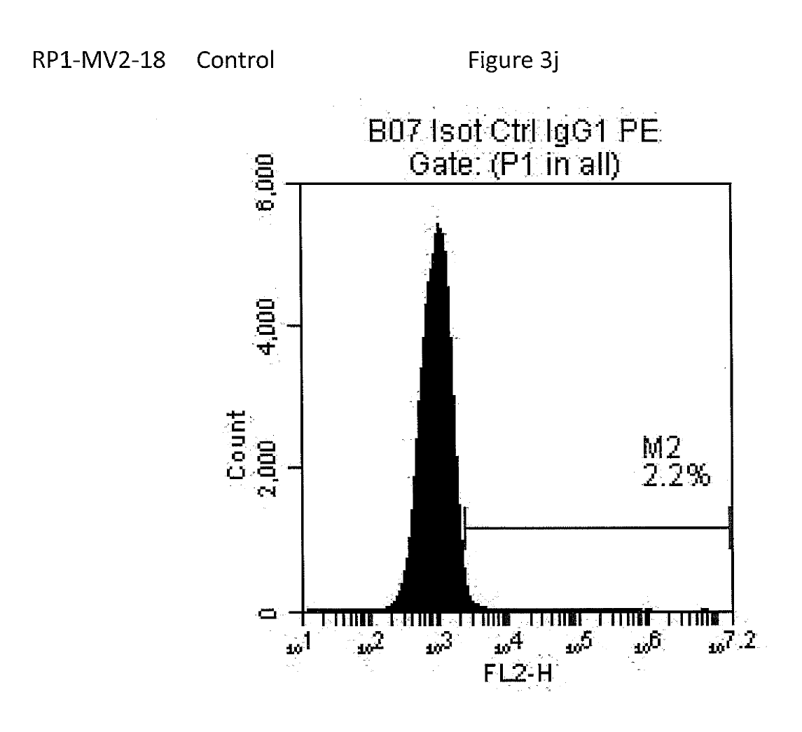

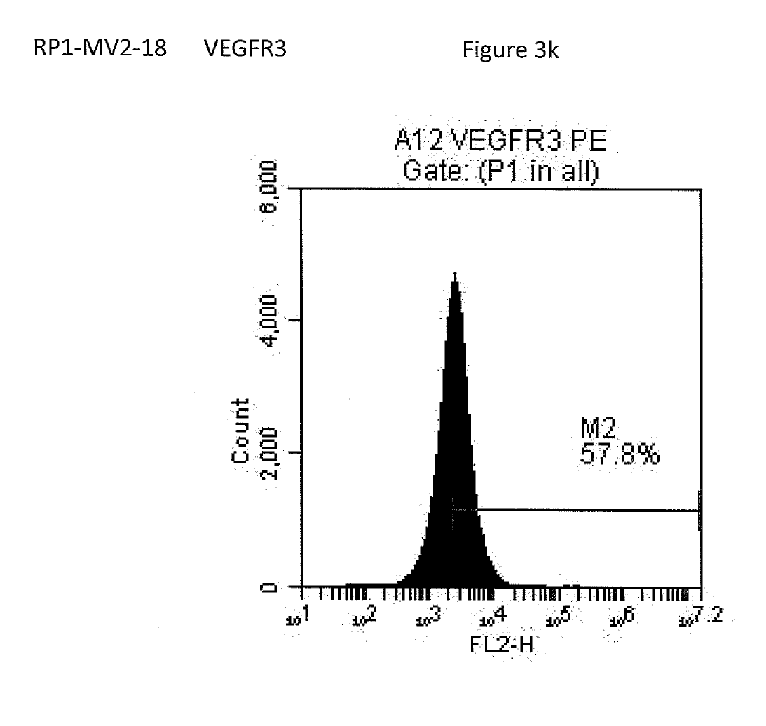

This heterogeneous population of hES-derived endothelial cells was compared to the clonally-derived endothelial cells of the present invention by Illumina gene expression microarray and by FACS. As shown in FIG. 2 and FIG. 3, the purity of the endothelial cells derived by each protocol as 17.8% of RP1-MV2-16, and determined by positivity for the endothelial cell marker was 54.7% pure for the heterogeneous culture, 99.9% pure for the monoclonal cell line RP1-MV2-6, 98.3% pure for the cell line RP1-MV2-16, and 100% pure for the cell line RP1-MV2-18. Whereas the CD34 expression was distributed in a biphasic manner in the heterogeneous culture, it was uniformly negative in the monoclonal cell lines. VE-Cadherin was positive in 54.6% of the heterogeneous cells, while positive in 99.3% of RP1-MV2-6 and 99.9% of RP1-MV2-18.

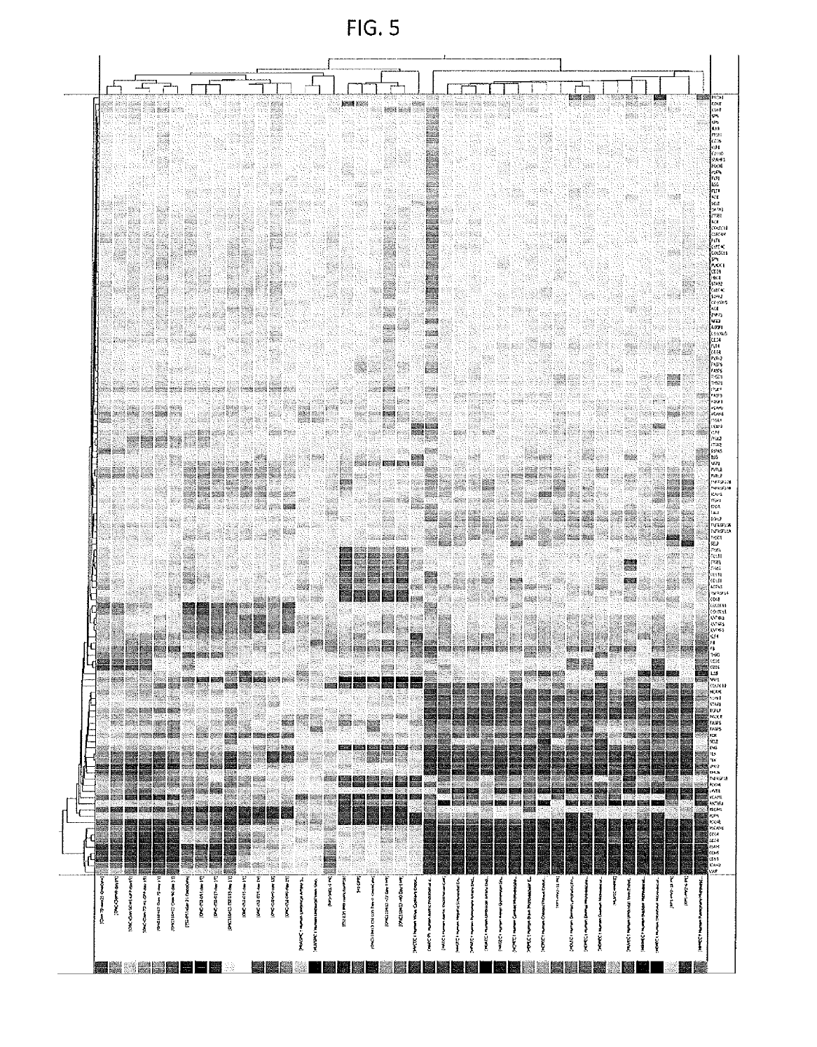

As shown in FIG. 5, the clonal cell lines RP1-MV2-6, RP1-MV2-16, and RP1-MV2-18 expressed higher levels of endothelial cell markers than did the heterogeneous endothelial cells such as the cells designated: ONC110413 ESI 035 day 11, ONC-ESI-017 day 15, ESI-051-day 21 OncoCyte. In addition, the clonal endothelial cell line of the present invention had a pattern of endothelial gene expression similar to diverse types of cultures of adult-derived normal endothelial cells. Genes with no or low expression are shown in yellow, genes with relatively high expression are shown in red.

### Example 3

### Initial Characterization of Myoblast Monoclonal Embryonic Progenitor Cell Lines Generated in the Presence of SB431542

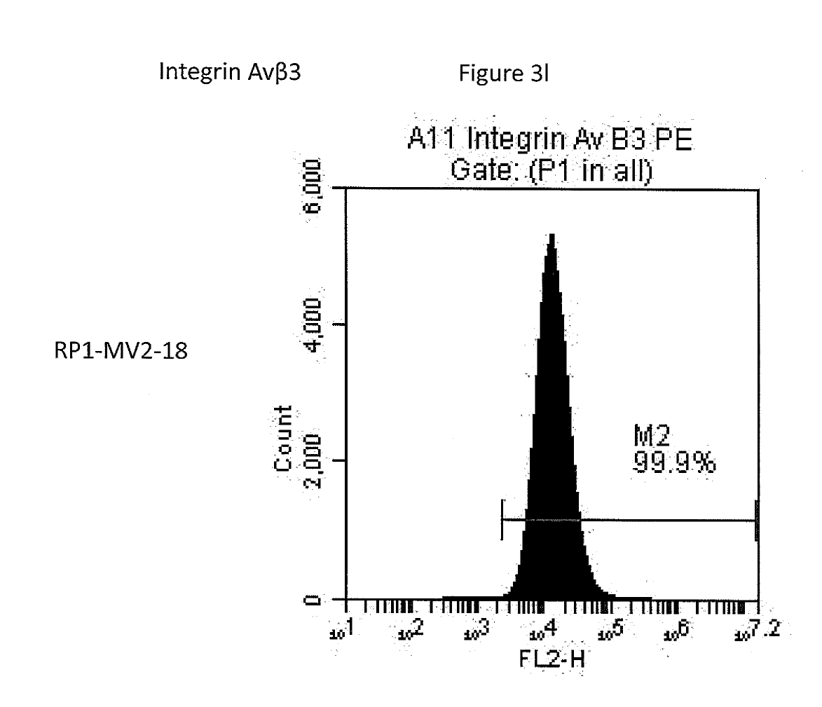

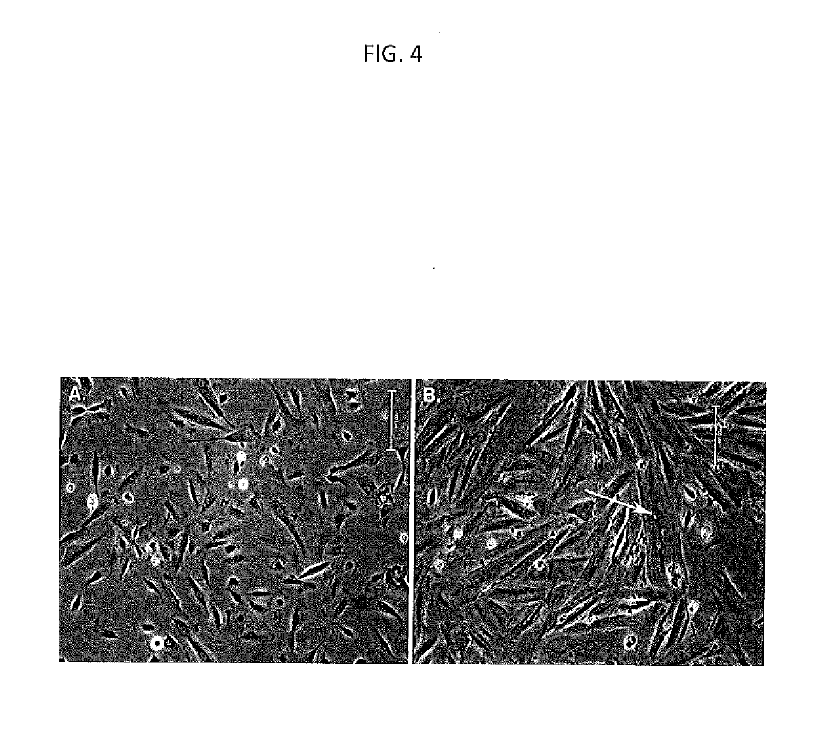

The cell line of the present invention RP 1-SKEL-8 was differentiated in DMEM media supplemented with 2.0% horse serum for 6 days. As shown in FIG. 4, abundant multinucleated cells appeared consistent with myocyte differentiation. The resulting differentiated cells were analyzed by immunocytochemistry for the presence of skeletal muscle cell markers including myogenin, ACTA1, and desmin.

### Example 4

### Reversion of hES-Derived Clonal Embryonic Progenitor Cell Lines to Cells with an Adipogenic Phenotype with SB431542

Existing clonal embryonic progenitor cell lines previously disclosed (see, e.g., U.S. patent application Ser. No. 11/604,047 filed on Nov. 21, 2006 and titled “Methods to Accelerate the Isolation of Novel Cell Strains from Pluripotent Stem Cells and Cells Obtained Thereby”; U.S. patent application Ser. No. 12/504,630 filed on Jul. 16, 2009 and titled “Methods to Accelerate the Isolation of Novel Cell Strains from Pluripotent Stem Cells and Cells Obtained Thereby”; and U.S. patent application Ser. No. 13/115,688 filed on May 25, 2011 and titled “Improved Methods of Screening Embryonic Progenitor Cell Lines”, each of which is incorporated herein by reference) that are capable of adipocyte differentiation may be differentiated into adipocytes by Adipogenesis Protocol 2 (below) combined with SB431542 as disclosed herein.

Adipogenesis Protocol 2
Cells are grown to confluence in their standard growth medium (West et al., 2008, Regenerative
Medicine vol. 3(3) pp. 287-308), medium is removed and replaced by serum-free differentiation
medium (DMEM/F12 containing 1 μM bovine insulin, 100 nM hydrocortisone, 10 μg of
transferrin/mL, 1 nM thyronine, 1 μM rosiglitazone, 33 μM biotin, and 17 μM pantothenic acid)
to induce adipocyte differentiation for 3 d. After 3 d of culture, the medium is changed to
differentiation medium without rosiglitazone for another 5 d. The mRNA from cultured cells was
extracted at 0, 2, 5, 7 and 14 d of incubation for transcript analysis as described herein.
Differentiation Factor Protocol 1
Cells are seeded in a 12 well plate precoated with fibronectin (Gibco) at a high density (1.5 × 10⁶
cells/well). Cells are fed three times per week for 14 days with a basal media of knock out
DMEM with penicillin/streptomycin and 16% knock out serum replacement. Individual
differentiation factors added to this basal medium chosen from Table III of U.S. Provisional
application Ser. No. 61/533,127 (the disclosure of which is herein incorporated by reference).
Control five day quiescent cells are plated at 3.0 × 10⁵ cells/well and at confluence fed media
with serum or other growth supplements reduced to 10% of normal values. The cells are refed
two days prior to harvest.

### Example 5

### Robust Derivation of Monoclonal Lineages of Purified PECAM1+, CDH5+, vWF+Endothelial Cell Lines from GMP-Capable hES Cells Including Lines Expressing ITLN1

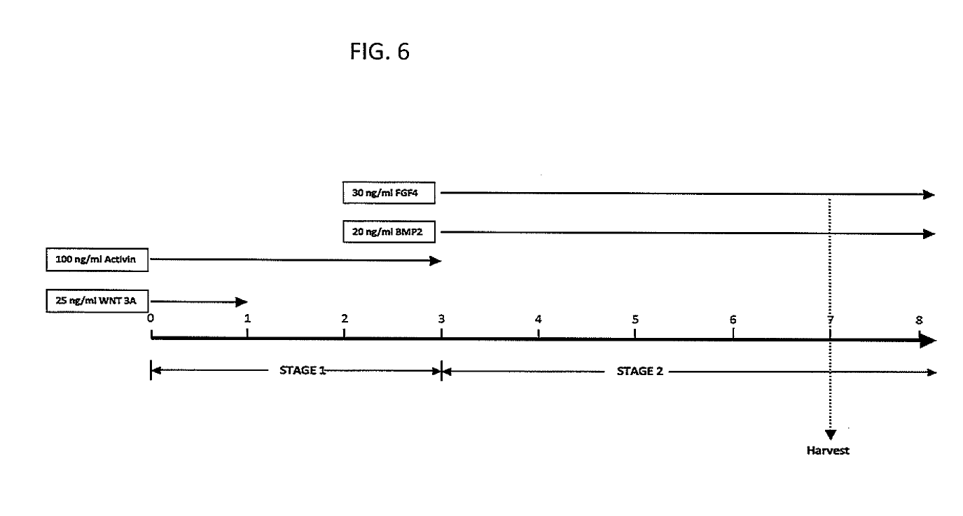

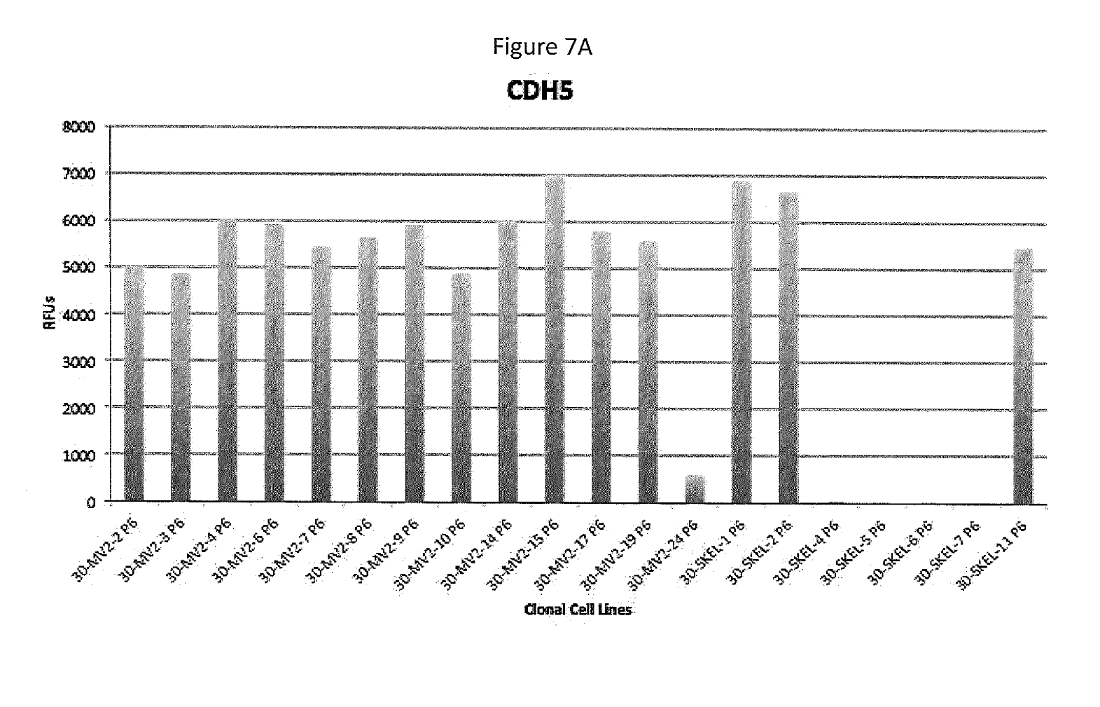

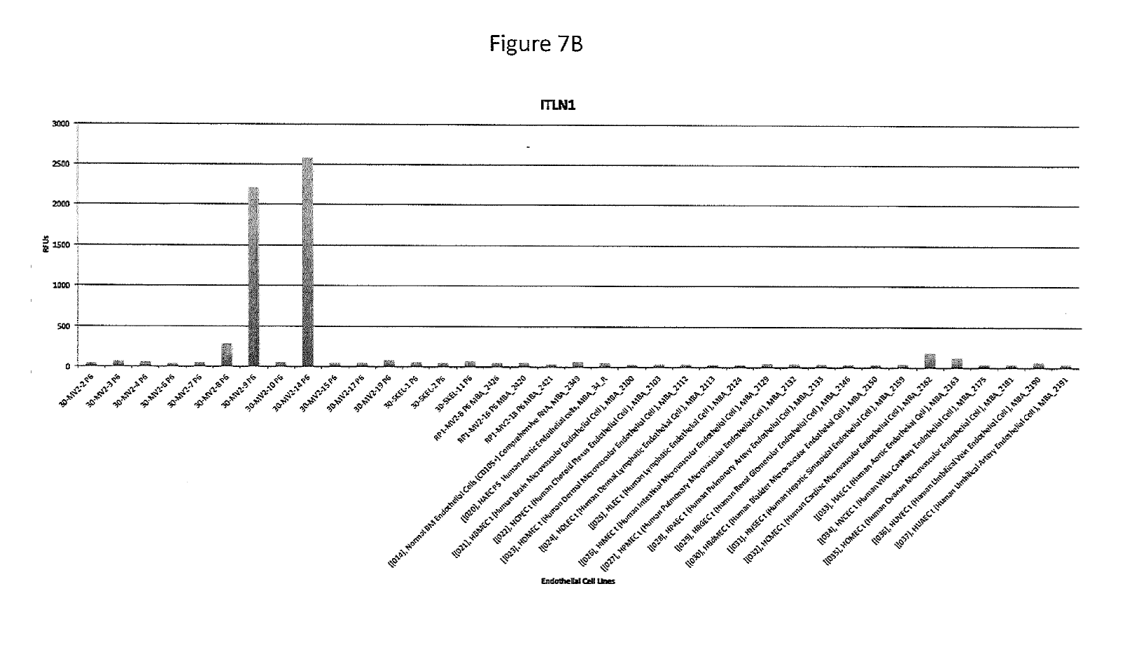

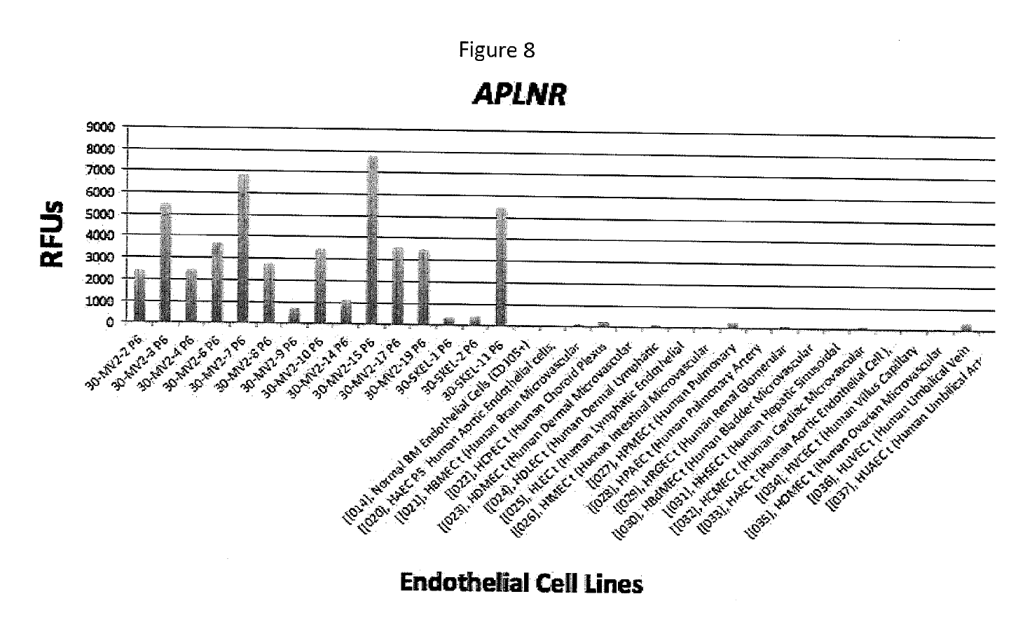

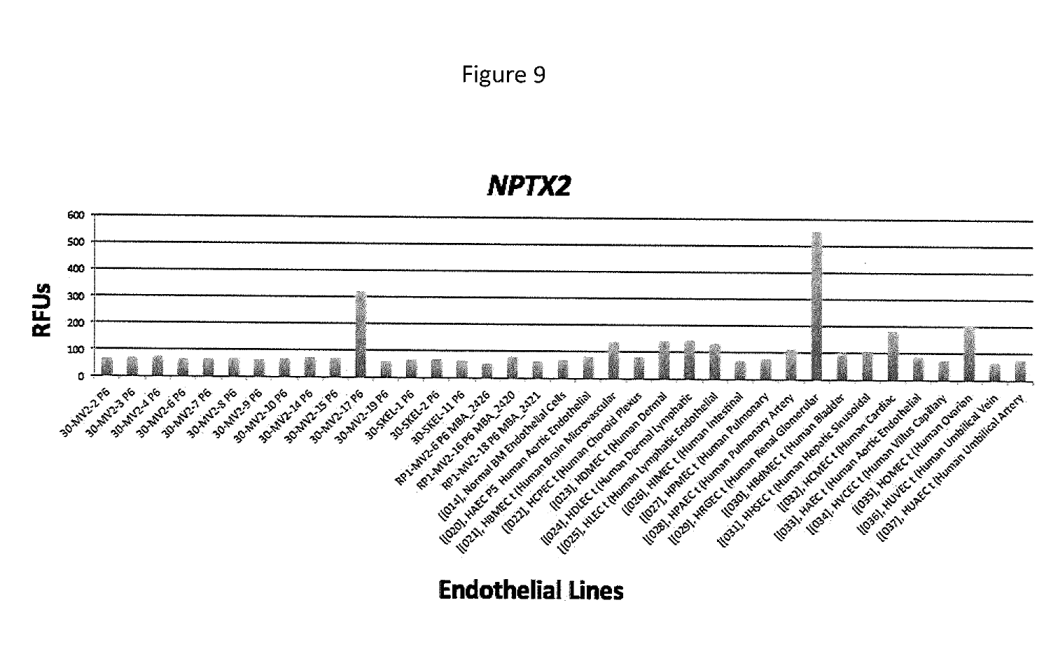

The hES cell line ESI-017 originally derived under cGMP conditions (Crook et al, Cell Stem Cell 1, November 2007), was differentiated in vitro on fibroblast feeder cells as colonies that are allowed to overgrow and differentiate in situ for 13 days in ES cell culture medium (Invitrogen KO-DMEM with KO-serum replacement). Then, on differentiation day 0 (FIG. 6), media was changed to a basal differentiation media comprising KO-DMEM/RPMI-1640 (5/1 v/v) and the basal differentiation media was supplemented with 100 ng/mL Activin A and 25 ng/mL Wnt3A. On the beginning of day 2 (designated Day 1 in FIG. 6), and for the following two days the media was replaced with the basal differentiation medium supplemented only with 100 ng/mL Activin A. Then on the beginning of day 4 (designated Day 3 on FIG. 6), the media was replaced with the basal differentiation media supplemented with 30 ng/mL FGF4 and 20 ng/mL BMP2. At the beginning of Day 8 (designated Day 7 in FIG. 6), cells were rinsed twice in PBS and disaggregated with Accutase, and plated on Matrigel-coated plates in Promocell endothelial MV2 media with supplements at concentrations recommended by the manufacturer and sold as a complete kit (Cat# C-22022) supplemented with 10 uM of the TGF signaling inhibitor SB431542. Cells were expanded as working stocks of candidate cultures that were expanded and cryopreserved for the purposes of deriving continuous clonal cell lines. The candidate cultures were plated at 500 and 2,000 cells in 15 cm tissue culture dishes coated with Matrigel in Promocell endothelial MV2 media with supplements at concentrations recommended by the manufacturer and sold as a complete kit (Cat# C-22022) supplemented with 10 uM of the TGFβ signaling inhibitor SB431542, and allowed to grow to visible cell colonies which are subsequently isolated by the use of cloning cylinders, and serially propagated as cell lines in the same media and supplements on Matrigel-coated tissue culture flasks. Of 13 isolated lines (30-MV2-2, 30-MV2-3, 30-MV2-4, 30-MV2-6, 30-MV2-7, 30-MV2-8, 30-MV2-9, 30-MV2-10, 30-MV2-14, 30-MV2-15, 30-MV2-17, 30-MV2-19, and 30-MV2-24) all but the line 30-MV2-24 assayed by Illumina microarrays, 12 or 13 lines were strongly positive for the expression of endothelial marker genes including: PECAM1, vWF, and CDH5 (VE-Cadherin) (FIG. 7A) (RFU values <70 being considered background signal and definitively lacking expression, 71-99 being indeterminate, >100 being considered positive), the line 30-MV2-24 being weakly positive for expression of the marker genes. Of these clonal lines, the lines 30-MV2-9 and 30-MV2-14 were strongly positive for the expression of ITLN1, ITLN2, POSTN, and MAMDC2 at passage 6. Analysis of normalized data from cultured endothelial cells from a number of normal tissues including human aortic, brain microvascular, choroid plexus, dermal microvascular, dermal lymphatic, intestinal microvascular, pulmonary microvascular, pulmonary artery, renal glomerular, bladder microvascular, hepatic sinusoidal, and other normal cultured endothelial cell types showed no or low levels of ITLN1 or ITLN2 gene expression (FIG. 7B,C) with the exception of the surprising result that cardiac microvascular endothelial cells expressed detectable levels of both ITLN1 and ITLN2 suggesting an important role of the protein in cardiac function, the need of maintaining sufficient levels of the protein to prevent disease, and the use of the cell lines of the present invention in restoring the levels of ITLN1 or ITLN2-encoded proteins to normal levels. In addition, the cell lines of the present invention express relatively high levels of genes of the apelin system (APLNR and APLN) (see FIG. 8). Since ischemia leads to activation of APLNR signaling in endothelium (Am J Physiol Heart Circ Physiol 294: H88-H98, 2008.), the cell lines of the present invention are ideally suited to express APLN and respond to said signaling in ischemic tissue, One skilled in the art will understand that the novel cell lines of the present invention also express other unique and useful markers, such as the line 30-MV2-17 that uniquely expresses NPTX2, a gene also expressed in cultured renal glomerular endothelial cells (FIG. 9). The protein has been reported to inhibit the growth of pancreatic tumor cells (Zhang L et al, 2011, Mol. Biol. Rep. Volume 38, Number 8, 4903-4911). Vascular endothelial cells expressing NPTX2 are useful in targeting pancreatic tumor angiogenesis and thereby inhibiting the growth of pancreatic cancer.

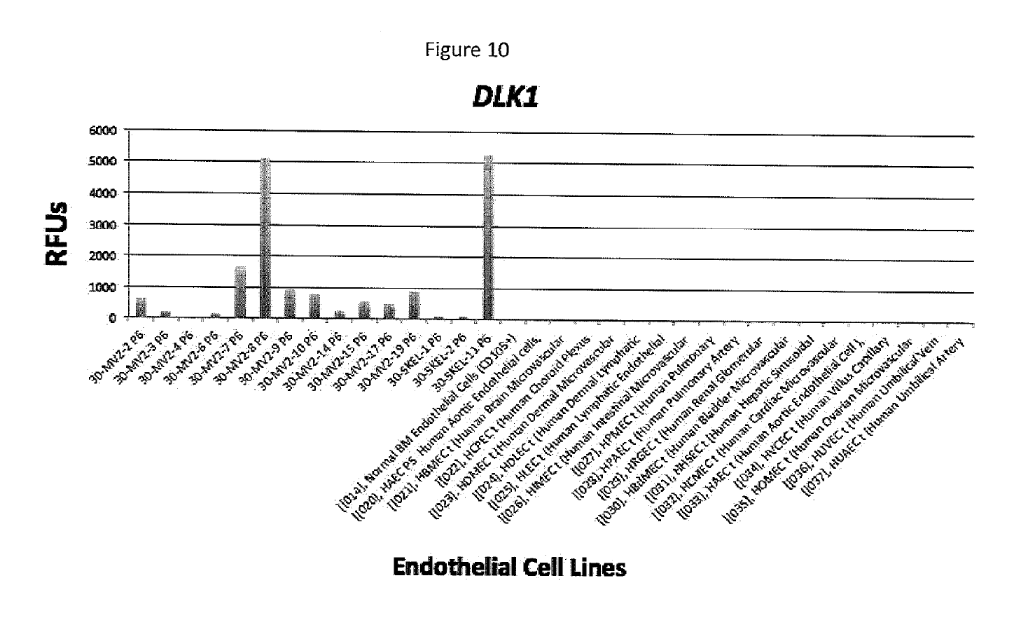

The cell lines thus isolated from hES or iPS cells are unique in that they display a prenatal pattern of gene expression (see e.g., PCT application serial no. PCT/US2006/013519 titled “Novel Uses of Cells With Prenatal Patterns of Gene Expression,” and published as WO 2007/058671, the disclosure of which is herein incorporated by reference). By way of non-limiting example, in the mouse it is reported that the gene DLK1 is abundantly expressed in the vascular endothelium of embryonic mice in vivo, while expression levels markedly decrease in late development or postnatally (Yevtodiyenko, A and Schmidt, J. V. 2006. Developmental Dyn. 235: 1115-1123). As can be seen in FIG. 10, DLK1 expression levels were low to undetectable in adult-derived endothelium such as that derived from normal tissues including: human aortic, brain microvascular, choroid plexus, dermal microvascular, dermal lymphatic, intestinal microvascular, pulmonary microvascular, pulmonary artery, renal glomerular, bladder microvascular, hepatic sinusoidal, and other normal cultured endothelial cell types, but was expressed at high levels in numerous clonal lines of the present invention. These novel lines therefore express genes and display properties never before attained in a purified and scalable state useful in research and therapy.

### Example 6

### Isolation of Additional Clonal Lineage Cell Lines

Additional cells differentiated under the conditions described in Example 1, were plated at clonal densities as described herein. RNA was obtained at passage 6 (six passages from the original isolated culture of a clonal population of cells) from cells cultured in the medium in which they were isolated but with a 10-fold reduction in growth factors for five days prior to RNA isolation in order to induce quiescence and thereby reduce variations in cell cycle-related genes. The RNA was analyzed by Illumina microarrays as described herein. In addition to vascular endothelium and myoblastic cells, other diverse clonal lines were obtained useful in research and therapy. One such line designated RP1-DM10-18 expressed the relatively rare gene expression markers SYT4 (Accession number NM_{—}020783.2), NPY (Accession number NM_{—}000905.2), FOXQ1 (Accession number NM_{—}033260.2), ALDH1A1 (Accession number NM_{—}000689.3), ALDH1A3 (Accession number NM_{—}000693.1), ATP8B4 (Accession number NM_{—}024837.2), NPPB (Accession number NM_{—}002521.1), FGF9 (Accession number NM_{—}002010.1), MKX (Accession number NM_{—}173576.1), PODN (Accession number NM_{—}153703.3), NXPH2 (Accession number NM_{—}007226.1), and the bradykinin receptor B1 (BDKRB1, Accession number NM_{—}000710.2). The expression of neuropeptide Y (NPY) by this cell line provides a novel means of both manufacturing the protein such as in conditioned medium or the urea extraction protocol described herein. The conditioned medium or urea extract may be used in research. The neuropeptide Y may be purified from the conditioned medium or extract using fractionation techniques known in the art or affinity purification e.g. using antibodies such as monoclonal antibodies. The protein may be administered using a means of continuously delivering the protein in a tissue in vivo in humans or other mammalian species to provide a therapeutic effect. Such therapeutic approaches include the use of this cell line or similar pluripotent stem cell-derived lines expressing the markers SYT4, NPY, FOXQ1, ALDH1A1, ALDH1A3, ATP8B4, NPPB, FGF9, MKX, PODN, NXPH2, and BDKRB1 for use in the treatment of refractory epilepsy where the therapeutic use of NPY is documented and where novel delivery systems is currently needed (Current Neuropharmacology, 2007, 5, 115-125).

Another clonal cell line isolated and analyzed under the conditions described in this example is designated RP1-DM10-19. At passage 6 this line expressed the gene expression markers: PCP4 (Accession number NM_{—}006198.2), ELA2A (Accession number NM_{—}033440.1), EGFL6 (Accession number NM_{—}015507.2), SYPL2 (Accession number NM_{—}001006603.1), and FOXQ1 (Accession number NM_{—}033260.2). The expression of EGFL6 by this cell line is an example of the isolation of a cell type displaying a prenatal pattern of gene expression since the gene is expressed in embryonic but not adult cell types (Genomics 62, 304-307 (1999)) and it provides a novel means of both manufacturing the protein e.g. using conditioned medium or the urea or deoxycholate extraction protocol described herein. EGF-like domain multiple 6 may be purified from the conditioned medium or extract using fractionation techniques known in the art or affinity purification e.g using antibodies such as monoclonal antibodies. The conditioned medium, extract, or purified protein may be used in research such as to increase the proliferation of adipocyte stromal fraction (Mol Cell Biochem. 2010 October; 343(1-2):257-69. Epub 2010 Jun. 25.), or as a means of continuously delivering the protein in a tissue in vivo in humans or other mammalian species to provide a therapeutic effect. Such therapeutic approaches include the use of this cell line or similar pluripotent stem cell-derived lines expressing the described markers for use in the treatment of hair loss by stimulating folliculogenesis (Experimental Cell Research 303 (2005) 148-159) where novel delivery systems is currently needed.

Another clonal cell line isolated and analyzed under the conditions described in this example is designated RP1-MV2-5. At passage 6 this line expressed the gene expression markers: IGFBP1 (Accession number NM_{—}001013029.1), SPOCK2 (Accession number NM_{—}014767.1), TRIM55 (Accession number NM_{—}184086.1), EREG (Accession number NM_{—}001432.1), NRCAM (Accession number NM_{—}005010.2), CHRDL1 (Accession number NM_{—}145234.2), and NKX2.5 (Accession number NM 004387.2). The expression of epiregulin (EREG) by this cell line provides a novel means of both manufacturing the protein using conditioned medium or the urea or deoxycholate extraction protocol described herein. Epiregulin may be purified from said conditioned medium or extract using fractionation techniques known in the art or affinity purification e.g. using antibodies such as monoclonal antibodies wherein the conditioned medium, extract, or purified protein is used in research such as to increase the proliferation of epithelial cell types such as cultured keratinocytes (J Biol Chem. 2000 Feb. 25; 275(8):5748-53.) or as a means of continuously delivering the protein in a tissue in vivo in humans or other mammalian species to provide a therapeutic effect. Such therapeutic approaches include the use of this cell line or similar pluripotent stem cell-derived lines expressing the described markers for use in the treatment of re-epithelialization and wound repair where novel delivery systems is currently needed.

Another clonal cell line isolated and analyzed under the conditions described in this example is designated RP1-MV2-8. At passage 6 this line expressed the gene expression markers: NPTX1 (Accession number NM_{—}002522.2), TRPC3 (Accession number NM_{—}003305.1), LGR6 (Accession number NM_{—}001017403.1), RASL11A (Accession number NM_{—}206827.1), DACH1 (Accession number NM_{—}080759.3), GATA2 (Accession number NM_{—}032638.3), and REN (Accession number NM_{—}000537.2). The expression of renin (REN) by this cell line provides a novel means of both manufacturing the protein using conditioned medium or the urea or deoxycholate extraction protocol described herein. Renin may be purified from the conditioned medium or extract using fractionation techniques known in the art or affinity purification e.g. using antibodies such as monoclonal antibodies. The conditioned medium, extract, or purified protein may be used in research such as to modulate blood pressure in experimental animals or as a means of continuously delivering the protein in a tissue in vivo in humans or other mammalian species to provide a therapeutic effect. Such therapeutic approaches include the use of this cell line or similar pluripotent stem cell-derived lines expressing the described markers for use in the treatment of blood pressure disorders resulting from renal failure and the loss of natural renin expression.

Another clonal cell line isolated and analyzed under the conditions described in this example is designated RP1-MV2-13. At passage 6 this line expressed the gene expression markers: CST1 (Accession number NM_{—}001898.2), MSMP (Accession number NM_{—}001044264.1), DIRAS3 (Accession number NM_{—}004675.2), ANXA8 (Accession number NM_{—}001040084.1), and CYP2S1 (Accession number NM_{—}030622.6). The expression of cystatin SN (CST1) by this cell line provides a novel means of both manufacturing the protein using conditioned medium or the urea or deoxycholate extraction protocol described herein. The renin may be purified from the conditioned medium or extract using fractionation techniques known in the art or affinity purification e.g. using antibodies such as monoclonal antibodies. The conditioned medium, extract, or purified protein is used in research such as to modulate blood pressure in experimental animals or as a means of continuously delivering the protein in a tissue in vivo in humans or other mammalian species to provide a therapeutic effect. Such therapeutic approaches include the use of this cell line or similar pluripotent stem cell-derived lines expressing the described markers such as cystatin SN for use in reducing the incidence of dental caries (Eur J Oral Sci. 2006 April; 114(2):147-53.) where novel means of continuously expression of the protein are needed.

Another clonal cell line isolated and analyzed under the conditions described in this example is designated ESI-EN02. At passage 6 this line expressed the gene expression markers: STMN2 (Accession number NM_{—}007029.2), BAPX1 (Accession number NM_{—}001189.2), BMP4 (Accession number NM_{—}130851.1), PITX1 (Accession number NM_{—}002653.3), and LYPD6B (Accession number NM_{—}177964.3). The expression of bone morphogenic protein 4 (BMP4) by this cell line provides a novel means of both manufacturing the protein using conditioned medium or the urea or deoxycholate extraction protocol described herein. BMP4 may be purified from said conditioned medium or extract using fractionation techniques known in the art or affinity purification e.g. using antibodies such as monoclonal antibodies. The conditioned medium, extract, or purified protein is used in research in differentiating stem cells or as a means of continuously delivering the protein in a tissue in vivo in humans or other mammalian species to provide a therapeutic effect. Such therapeutic approaches include the use of this cell line or similar pluripotent stem cell-derived lines expressing the described markers for use in decreasing tumor load in patients with colon cancer (Cancer Biology & Therapy 3:7 667-675 (2004)).

Another clonal cell line isolated and analyzed under the conditions described in this example is designated ESI-SK43. At passage 6 this line expressed the gene expression markers: GNRH2 (Accession number NM_{—}001501.1), TUBA3D (Accession number NM_{—}080386.1), TUBA3E (Accession number NM 207312.1), ARTS (Accession number 053017.2), and MS4A8B (Accession number NM_{—}031457.1). The expression of gonadotropin releasing hormone 2 (GNRH2) by this cell line provides a novel means of both manufacturing the protein using conditioned medium or the urea or deoxycholate extraction protocol described herein. GNRH2 may be purified from the conditioned medium or extract using fractionation techniques known in the art or affinity purification using antibodies such as monoclonal antibodies wherein the conditioned medium, extract, or purified protein is used in research in endocrinology or as a means of continuously delivering the protein in a tissue in vivo in humans or other mammalian species to provide a therapeutic effect. Such therapeutic approaches include the use of this cell line or similar pluripotent stem cell-derived lines expressing the described markers for use in modulating reproductive and feeding behaviors in humans and non-human mammals.

Another clonal cell line isolated and analyzed under the conditions described in this example is designated ESI-SK01. At passage 6 this line expressed the gene expression markers: SFTPD (Accession number NM_{—}003019.4), SPRR2F (Accession number NM 001014450.1), SLPI (Accession number NM_{—}003064.2), SEPP1 (Accession number 005410.2), and NPNT (Accession number NM_{—}001033047.1). The expression of surfactant protein D (SFTPD) by this cell line provides a novel means of both manufacturing the protein using conditioned medium or the urea or deoxycholate extraction protocol described herein. SFTPD is purified from said conditioned medium or extract using fractionation techniques known in the art or affinity purification such as by means of antibodies e.g. using monoclonal antibodies wherein the conditioned medium, extract, or purified protein is used in research in lung and surfactant function or as a means of continuously delivering the protein in a tissue in vivo in humans or other mammalian species to provide a therapeutic effect. Such therapeutic approaches include the use of this cell line or similar pluripotent stem cell-derived lines expressing the described markers for use in increasing surfactant function in humans such as is needed in preterm infants with relatively low surfactant levels.

Another clonal cell line isolated and analyzed under the conditions described in this example is designated ESI-SK01. At passage 6 this line expressed the gene expression markers: SFTPD (Accession number NM_{—}003019.4), SPRR2F (Accession number NM 001014450.1), SLPI (Accession number NM_{—}003064.2), SEPP1 (Accession number 005410.2), and NPNT (Accession number NM_{—}001033047.1). The expression of surfactant protein D (SFTPD) by this cell line provides a novel means of both manufacturing the protein using conditioned medium or the urea or deoxycholate extraction protocol described herein. SFTPD may be purified from the conditioned medium or extract using fractionation techniques known in the art or affinity purification e.g. using antibodies such as monoclonal antibodies. The conditioned medium, extract, or purified protein may be used in research in lung and surfactant function or as a means of continuously delivering the protein in a tissue in vivo in humans or other mammalian species to provide a therapeutic effect. Such therapeutic approaches include the use of this cell line or similar pluripotent stem cell-derived lines expressing the described markers for use in increasing surfactant fiunction in humans such as is needed in preterm infants with relatively low surfactant levels.

TABLE I
Culture Conditions

| | |
|---|---|
| 1. | Subconfluent Monolayer Culture: Cells are plated and exposed to combinations of the conditions listed in |
| | Tables I-IV herein while said cells are in a subconcluent state. |
| 2. | Confluent Monolayer Culture: Cells are plated and exposed to combinations of the conditions listed in |
| | Tables I-IV herein while said cells are in a confluent monolayer state. |
| 3. | Micromass Culture: Cells are plated and exposed to combinations of the conditions listed in Tables I-IV |
| | herein while said cells are in a highly dense micromass state as described herein. |
| 4. | Subconfluent Mixed Culture: Cells are plated and exposed to combinations of the conditions listed in |
| | Tables I-IV herein while said cells are in a subconfluent state and juxtasposed (co-cultured) potentially in |
| | physical contact with cells of another differentiated state or another distinguishable cell line of the present |
| | invention. |
| 5. | Subconfluent Transwell Culture: Cells are plated and exposed to combinations of the conditions listed in |
| | Tables I-IV herein while said cells are in transwell vessels or tissue cultureware of similar design that |
| | allows the physical separation of diverse cell types but allowing a sharing of their media. Such |
| | subconfluent transwell culture is where the cell lines of the present invention are subconfluent and share |
| | culture media with a cell type of a different differentiated state wherein the cells of a different differentiated |
| | state may be themselves in a subconfluent or confluent state. |
| 6. | Confluent Mixed Culture: Cells are plated and exposed to combinations of the conditions listed in Tables I- |
| | IV herein while said cells are in a confluent state and juxtasposed (co-cultured) potentially in physical |
| | contact with cells of another differentiated state or another distinguishable cell line of the present invention. |
| 7. | Confluent Transwell Culture: Cells are plated and exposed to combinations of the conditions listed in |
| | Tables I-IV herein while said cells are in transwell vessels or tissue cultureware of similar design that |
| | allows the physical separation of diverse cell types but allowing a sharing of their media. Such |
| | subconfluent transwell culture is where the cell lines of the present invention are confluent and share |
| | culture media with a cell type of a different differentiated state wherein the cells of a different differentiated |
| | state may be themselves in a subconfluent or confluent state. |
| 8. | Micromass Mixed Culture: Cells are plated and exposed to combinations of the conditions listed in Tables |
| | I-IV herein while said cells are in a highly dense micromass state as described herein and juxtasposed (co- |
| | cultured) potentially in physical contact with cells of another differentiated state or another distinguishable |
| | cell line of the present invention. |
| 9. | Micromass Transwell Culture: Cells are plated and exposed to combinations of the conditions listed in |
| | Tables I-IV herein while said cells are in transwell vessels or tissue cultureware of similar design that |
| | allows the physical separation of diverse cell types but allowing a sharing of their media while said cells |
| | are in a highly dense micromass state as described herein. Such subconfluent transwell culture is where the |
| | cell lines of the present invention are confluent and share culture media with a cell type of a different |
| | differentiated state wherein the cells of a different differentiated state may be themselves in a subconfluent |
| | or confluent state. |

Culture Exposed to Cell Extracts of Cells of a Different Differentiated State: Target cells are plated and exposed to combinations of the conditions listed in Tables I-IV herein while said cells are in a subconfluent state and wherein the media for said cells contains extracts of cells of a differing differentiated state and wherein said target cells are exposed to conditions that facilitate the intracellular trafficking of molecules such as described in U.S. patent application Ser. No. 10/910,156 filed on Aug. 2, 2004 and titled “Methods for Altering Cell Fate”, and U.S. patent application Ser. No. 10/015,824 filed on Dec. 10, 2001 and titled “Methods for Altering Cell Fate”, both incorporated herein by reference in their entirety.

TABLE II
Culture Media and Related Culture Variables
Culture Media

| | |
|---|---|
| 1) | DMEM (Dulbecco's Modified Eagle's Medium). HyClone Cat. No. |
| | SH30285.03 |
| 2) | Airway Epithelial Growth Medium (PromoCell Cat. No. C-21260 |
| | with supplement Cat No. C-39160) |
| 3) | Epi-Life (LSGS) Medium (Cascade Cat. No. M-EPIcf/PRF-500 |
| | with supplement Cat. No. S-003-10) |
| 4) | Neural Basal Medium B-27 (Gibco Cat. No. 12348-017 with B-27 |
| | supplement Cat. No. 12587-010) |
| 5) | Neural Basal Medium N-2 (Gibco Cat. No. 12348-017 with N-2 |
| | supplement Cat. No. 17502-048) |
| 6) | HepatoZyme-SFM (Gibco Cat. No. 17705-021) |
| 7) | Epi-Life (HKGS) Medium (Cascade Cat. No. M EPIcf/PRF-500 |
| | with supplement Cat. No. S-001-5) |
| 8) | Endothelial Cell Growth Medium (PromoCell Cat. No. C-22221 |
| | with supplement Cat No. C-39221) |
| 9) | Endothelial Cell SFM (Gibco Cat. No. 11111-044 with basic |
| | fibroblast growth factor Cat. No. 13256-029, epidermal growth |
| | factor Cat. No. 13247-051 and fibronectin Cat. No. 33016-015) |
| 10) | Skeletal Muscle Medium (PromoCell Cat. No. C-23260 |
| | with supplement Cat. No. C-39360) |
| 11) | Smooth Muscle Basal Medium (PromoCell Cat. No. C-22262 |
| | with supplement Cat. No. C-39262) |
| 12) | MesenCult Medium (Stem Cell Technologies Cat. No. 05041 |
| | with supplement Cat. No. 05402) |
| 13) | Melanocyte Growth Medium (PromoCell Cat. No. C 24010 with |
| | supplement Cat. No. C-39410) |
| 14) | Ham's F-10 Medium |
| 15) | Ham's F-12 Medium |
| 16) | DMEM/Ham's F-12 50/50 mix |
| 17) | Iscove's Modified Dulbecco's Medium (IMDM) |
| 18) | Leibovitz's L-15 Medium |
| 19) | McCoy's 5A Medium Modified |
| 20) | RPMI 1640 Medium |
| 21) | Glasgow's MEM (GMEM) |
| 22) | Eagle's Medium |
| 23) | Medium 199 |
| 24) | MEM Eagle-Earle's |

Antibiotics

| | |
|---|---|
| 25) | Penicillin |
| 26) | Streptomycin |
| 27) | Gentamycin |
| 28) | Neomycin |
| 29) | G418 |

Other Factors

| | |
|---|---|
| 30) | Human plasma |
| 31) | Chick embryo extract |
| 32) | Human plasmanate |

TABLE III
Supplemented Factors
EGF Ligands

| | |
|---|---|
| 1) | Amphiregulin |
| 2) | Betacellulin |
| 3) | EGF |
| 4) | Epigen |
| 5) | Epiregulin |
| 6) | HB-EGF |
| 7) | Neuregulin-3 |
| 8) | NRG1 isoform GGF2 |
| 9) | NRG1 Isoform SMDF |
| 10) | NRG1-alpha/HRG1-alpha |
| 11) | TGF-alpha |
| 12) | TMEFF1/Tomoregulin-1 |
| 13) | TMEFF2 |
| 14) | EGF Ligands pooled (1-13 above) |

EGF R/ErbB Receptor Family

| | |
|---|---|
| 15) | EGF Receptor |
| 16) | ErbB2 |
| 17) | ErbB3 |
| 18) | ErbB4 |
| 19) | EGF/ErbB Receptors pooled (15-18 above) |

FGF Ligands

| | |
|---|---|
| 20) | FGF acidic |
| 21) | FGF basic |
| 22) | FGF-3 |
| 23) | FGF-4 |
| 24) | FGF-5 |
| 25) | FGF-6 |
| 26) | KGF/FGF-7 |
| 27) | FGF-8 |
| 28) | FGF-9 |
| 29) | FGF-10 |
| 30) | FGF-11 |
| 31) | FGF-12 |
| 32) | FGF-13 |
| 33) | FGF-14 |
| 34) | FGF-15 |
| 35) | FGF-16 |
| 36) | FGF-17 |
| 37) | FGF-18 |
| 38) | FGF-19 |
| 39) | FGF-20 |
| 40) | FGF-21 |
| 41) | FGF-22 |
| 42) | FGF-23 |
| 43) | FGF Ligands pooled (20-38 above) |

FGF Receptors

| | |
|---|---|
| 40) | FGF R1 |
| 41) | FGF R2 |
| 42) | FGF R3 |
| 43) | FGF R4 |
| 44) | FGF R5 |
| 45) | FGF Receptors pooled (40-44 above) |

FGF Regulators

| | |
|---|---|
| 46) | FGF-BP |

Hedgehogs

| | |
|---|---|
| 47) | Desert Hedgehog |
| 48) | Sonic Hedgehog |
| 49) | Indian Hedgehog |
| 50) | Hedgehogs pooled (47-49 above) |

Hedgehog Regulators

| | |
|---|---|
| 51) | Gas1 |
| 52) | Hip |
| 53) | Hedgehog Regulators pooled (51-52 above) |

IGF Ligands

| | |
|---|---|
| 54) | IGF-I |
| 55) | IGF-II |
| 56) | IGF Ligands pooled (54-55 above) |

IGF-I Receptor (CD221)

| | |
|---|---|
| 57) | IGF-I R |

GF Binding Protein (IGFBP) Family

| | |
|---|---|
| 58) | ALS |
| 59 | IGFBP-4 |
| 60) | CTGF/CCN2 |
| 61) | IGFBP-5 |
| 62) | Endocan |
| 63) | IGFBP-6 |
| 64) | IGFBP-1 |
| 65) | IGFBP-rp1/IGFBP-7 |
| 66) | IGFBP-2 |
| 67) | NOV/CCN3 |
| 68) | IGFBP-3 |
| 69) | GF Binding Protein Family pooled (58-68 above) |

Receptor Tyrosine Kinases

| | |
|---|---|
| 70) | Axl |
| 71) | C1q R1/CD93 |
| 72) | DDR1 |
| 73) | Flt-3 |
| 74) | DDR2 |
| 75) | HGF R |
| 76) | Dtk |
| 77) | IGF-II R |
| 78) | Eph |
| 79) | Insulin R/CD220 |
| 80) | EphA1 |
| 81) | M-CSF R |
| 82) | EphA2 |
| 83) | Mer |
| 84) | EphA3 |
| 85) | MSP R/Ron |
| 86) | EphA4 |
| 87) | MuSK |
| 88) | EphA5 |
| 89) | PDGF R alpha |
| 90) | EphA6 |
| 91) | PDGF R beta |
| 92) | EphA7 |
| 93) | Ret |
| 94) | EphA8 |
| 95) | ROR1 |
| 96) | EphB1 |
| 97) | ROR2 |
| 98) | EphB2 |
| 99) | SCF R/c-kit |
| 100) | EphB3 |
| 101) | Tie-1 |
| 102) | EphB4 |
| 103) | Tie-2 |
| 104) | EphB6 |
| 105) | TrkA |
| 106) | TrkB |
| 107) | TrkC |
| 108) | VEGF R1/Flt-1 |
| 109) | VEGF R2/Flk-1 |
| 110) | VEGF R3/Flt-4 |
| 111) | Receptor Tyrosine Kinases pooled (70-110 above) |

Proteoglycans

| | |
|---|---|
| 112) | Aggrecan |
| 113) | Lumican |
| 114) | Biglycan |
| 115) | Mimecan |
| 116) | Decorin |
| 117) | NG2/MCSP |
| 118) | Endocan |
| 119) | Osteoadherin |
| 120) | Endorepellin |
| 121) | Syndecan-1/CD138 |
| 122) | Glypican 2 |
| 123) | Syndecan-3 |
| 124) | Glypican 3 |
| 125) | Testican 1/SPOCK1 |
| 126) | Glypican 5 |
| 127) | Testican 2/SPOCK2 |
| 128) | Glypican 6 |
| 129) | Testican 3/SPOCK3 |
| 130) | Heparan sulfate proteoglycan |
| 131) | Heparin |
| 132) | Chondroitin sulfate proteoglycan |
| 133) | Hyaluronic acid |
| 134) | Dermatan sulfate proteoglycan |

Proteoglycan Regulators

| | |
|---|---|
| 135) | Arylsulfatase A/ARSA |
| 136) | HAPLN1 |
| 137) | Exostosin-like 2 |
| 138) | HS6ST2 |
| 139) | Exostosin-like 3 |
| 140) | IDS |
| 141) | Proteoglycan Regulators pooled (135-140 above) |

SCF, Flt-3 Ligand & M-CSF

| | |
|---|---|
| 142) | Flt-3 |
| 143) | M-CSF R |
| 144) | Flt-3 Ligand |
| 145) | SCF |
| 146) | M-CSF |
| 147) | SCF R/c-kit |
| 148) | Pooled factors (142-147 above) |

Activins

| | |
|---|---|
| 149) | Activin A |
| 150) | Activin B |
| 151) | Activin AB |
| 152) | Activin C |
| 153) | Pooled Activins (149-152 above) |

BMPs (Bone Morphogenetic Proteins)

| | |
|---|---|
| 154) | BMP-2 |
| 155) | BMP-3 |
| 156) | BMP-3b/GDF-10 |
| 157) | BMP-4 |
| 158) | BMP-5 |
| 159) | BMP-6 |
| 160) | BMP-7 |
| 161) | BMP-8 |
| 162) | Decapentaplegic |
| 163) | Pooled BMPs (154-162 above) |

GDFs (Growth Differentiation Factors)

| | |
|---|---|
| 164) | GDF-1 |
| 165) | GDF-2 |
| 166) | GDF-3 |
| 167) | GDF-4 |
| 168) | GDF-5 |
| 169) | GDF-6 |
| 170) | GDF-7 |
| 171) | GDF-8 |
| 172) | GDF-9 |
| 173) | GDF-10 |
| 174) | GDF-11 |
| 175) | GDF-12 |
| 176) | GDF-13 |
| 177) | GDF-14 |
| 178) | GDF-15 |
| 179) | GDFs pooled (164-178 above) |

GDNF Family Ligands

| | |
|---|---|
| 180) | Artemin |
| 181) | Neurturin |
| 182) | GDNF |
| 183) | Persephin |
| 184) | GDNF Ligands pooled (180-183 above) |

TGF-beta

| | |
|---|---|
| 185) | TGF-beta |
| 186) | TGF-beta 1 |
| 187) | TGF-beta 1.2 |
| 188) | TGF-beta 2 |
| 189) | TGF-beta 3 |
| 190) | TGF-beta 4 |
| 191) | TGF-beta 5 |
| 192) | LAP (TGF-beta 1) |
| 193) | Latent TGF-beta 1 |
| 194) | TGF-beta pooled (185-193 above) |

Other TGF-beta Superfamily Ligands

| | |
|---|---|
| 195) | Lefty |
| 196) | Nodal |
| 197) | MIS/AMH |
| 198) | Other TGF-beta Ligands pooled (195-197 above) |

TGF-beta Superfamily Receptors

| | |
|---|---|
| 199) | Activin RIA/ALK-2 |
| 200) | GFR alpha-1 |
| 201) | Activin RIB/ALK-4 |
| 202) | GFR alpha-2 |
| 203) | Activin RIIA |
| 204) | GFR alpha-3 |
| 205) | Activin RIIB |
| 206) | GFR alpha-4 |
| 207) | ALK-1 |
| 208) | MIS RII |
| 209) | ALK-7 |
| 210) | Ret |
| 211) | BMPR-IA/ALK-3 |
| 212) | TGF-beta RI/ALK-5 |
| 213) | BMPR-IB/ALK-6 |
| 214) | TGF-beta RII |
| 215) | BMPR-II |
| 216) | TGF-beta RIIb |
| 217) | Endoglin/CD105 |
| 218) | TGF-beta RIII |
| 219) | TGF-beta family receptors pooled (199-218 above) |

TGF-beta Superfamily Modulators

| | |
|---|---|
| 220) | Amnionless |
| 221) | GASP-2/WFIKKN |
| 222) | BAMBI/NMA |
| 223) | Gremlin |
| 224) | Caronte |
| 225) | NCAM-1/CD56 |
| 226) | Cerberus 1 |
| 227) | Noggin |
| 228) | Chordin |
| 229) | PRDC |
| 230) | Chordin-Like 1 |
| 231) | Chordin-Like 2 |
| 232) | Smad1 |
| 233) | Smad4 |
| 234) | Smad5 |
| 235) | Smad7 |
| 236) | Smad8 |
| 237) | CRIM1 |
| 238) | Cripto |
| 239) | Crossveinless-2 |
| 240) | Cryptic |
| 241) | SOST |
| 242) | DAN |
| 243) | Latent TGF-beta bp1 |
| 244) | TMEFF1/Tomoregulin-1 |
| 245) | FLRG |
| 246) | TMEFF2 |
| 247) | Follistatin |
| 248) | TSG |
| 249) | Follistatin-like 1 |
| 250) | Vasorin |
| 251) | GASP-1/WFIKKNRP |
| 252) | TGF Modulators pooled (220-251 above) |

VEGF/PDGF Family

| | |
|---|---|
| 253) | Neuropilin-1 |
| 254) | PlGF |
| 255) | PlGF-2 |
| 256) | Neuropilin-2 |
| 257) | PDGF |
| 258) | VEGF R1/Flt-1 |
| 259) | PDGF R alpha |
| 260) | VEGF R2/Flk-1 |
| 261) | PDGF R beta |
| 262) | VEGF R3/Flt-4 |
| 263) | PDGF-A |
| 264) | VEGF |
| 265) | PDGF-B |
| 266) | VEGF-B |
| 267) | PDGF-C |
| 268) | VEGF-C |
| 269) | PDGF-D |
| 270) | VEGF-D |
| 271) | PDGF-AB |
| 272) | VEGF/PDGF Family pooled (253-271 above) |

Dickkopf Proteins & Wnt Inhibitors

| | |
|---|---|
| 273) | Dkk-1 |
| 274) | Dkk-2 |
| 275) | Dkk-3 |
| 276) | Dkk-4 |
| 277) | Soggy-1 |
| 278) | WIF-1 |
| 279) | Pooled factors (273-278 above) |

Frizzled & Related Proteins

| | |
|---|---|
| 280) | Frizzled-1 |
| 281) | Frizzled-2 |
| 282) | Frizzled-3 |
| 283) | Frizzled-4 |
| 284) | Frizzled-5 |
| 285) | Frizzled-6 |
| 286) | Frizzled-7 |
| 287) | Frizzled-8 |
| 288) | Frizzled-9 |
| 289) | sFRP-1 |
| 290) | sFRP-2 |
| 291) | sFRP-3 |
| 292) | sFRP-4 |
| 293) | MFRP |
| 294) | Factors pooled (280-293 above) |

Wnt Ligands

| | |
|---|---|
| 295) | Wnt-1 |
| 296) | Wnt-2 |
| 297) | Wnt-3 |
| 298) | Wnt-3a |
| 299) | Wnt-4 |
| 300) | Wnt-5 |
| 301) | Wnt-5a |
| 302) | Wnt-6 |
| 303) | Wnt-7 |
| 304) | Wnt-8 |
| 305) | Wnt-8a |
| 306) | Wnt-9 |
| 307) | Wnt-10a |
| 308) | Wnt-10b |
| 309) | Wnt-11 |
| 310 | Wnt Ligands pooled (295-309 above) |

Other Wnt-related Molecules

| | |
|---|---|
| 311) | beta-Catenin |
| 312) | LRP-6 |
| 313) | GSK-3 |
| 314) | ROR1 |
| 315) | Kremen-1 |
| 316) | ROR2 |
| 317) | Kremen-2 |
| 318) | WISP-1/CCN4 |
| 319) | LRP-1 |
| 320) | Pooled factors (311-319 above) |

Other Growth Factors

| | |
|---|---|
| 321) | CTGF/CCN2 |
| 322) | NOV/CCN3 |
| 323) | EG-VEGF/PK1 |
| 324) | Osteocrin |
| 325) | Hepassocin |
| 326) | PD-ECGF |
| 327) | HGF |
| 328) | Progranulin |
| 329) | beta-NGF |
| 330) | Thrombopoietin |
| 331) | Pooled factors (321-330 above) |

Steroid Hormones

| | |
|---|---|
| 332) | 17beta-Estradiol |
| 333) | Testosterone |
| 334) | Cortisone |
| 335) | Dexamethasone |

Extracellular/Membrane Proteins

| | |
|---|---|
| 336) | Plasma Fibronectin |
| 337) | Tissue Fibronectin |
| 338) | Fibronectin fragments |
| 339) | Collagen Type I (gelatin) |
| 340) | Collagen Type II |
| 341) | Collagen Type III |
| 342) | Tenascin |
| 343) | Matrix Metalloproteinase 1 |
| 344) | Matrix Metalloproteinase 2 |
| 345) | Matrix Metalloproteinase 3 |
| 346) | Matrix Metalloproteinase 4 |
| 347) | Matrix Metalloproteinase 5 |
| 348) | Matrix Metalloproteinase 6 |
| 349) | Matrix Metalloproteinase 7 |
| 350) | Matrix Metalloproteinase 8 |
| 351) | Matrix Metalloproteinase 9 |
| 352) | Matrix Metalloproteinase 10 |
| 353) | Matrix Metalloproteinase 11 |
| 354) | Matrix Metalloproteinase 12 |
| 355) | Matrix Metalloproteinase 13 |
| 356) | ADAM-1 |
| 357) | ADAM-2 |
| 358) | ADAM-3 |
| 359) | ADAM-4 |
| 360) | ADAM-5 |
| 361) | ADAM-6 |
| 362) | ADAM-7 |
| 363) | ADAM-8 |
| 364) | ADAM-9 |
| 365) | ADAM-10 |
| 366) | ADAM-11 |
| 367) | ADAM-12 |
| 368) | ADAM-13 |
| 369) | ADAM-14 |
| 370) | ADAM-15 |
| 371) | ADAM-16 |
| 372) | ADAM-17 |
| 373) | ADAM-18 |
| 374) | ADAM-19 |
| 375) | ADAM-20 |
| 376) | ADAM-21 |
| 377) | ADAM-22 |
| 378) | ADAM-23 |
| 379) | ADAM-24 |
| 380) | ADAM-25 |
| 381) | ADAM-26 |
| 382) | ADAM-27 |
| 383) | ADAM-28 |
| 384) | ADAM-29 |
| 385) | ADAM-30 |
| 386) | ADAM-31 |
| 387) | ADAM-32 |
| 388) | ADAM-33 |
| 389) | ADAMTS-1 |
| 390) | ADAMTS-2 |
| 391) | ADAMTS-3 |
| 392) | ADAMTS-4 |
| 393) | ADAMTS-5 |
| 394) | ADAMTS-6 |
| 395) | ADAMTS-7 |
| 396) | ADAMTS-8 |
| 397) | ADAMTS-9 |
| 398) | ADAMTS-10 |
| 399) | ADAMTS-11 |
| 400) | ADAMTS-12 |
| 401) | ADAMTS-13 |
| 402) | ADAMTS-14 |
| 403) | ADAMTS-15 |
| 404) | ADAMTS-16 |
| 405) | ADAMTS-17 |
| 406) | ADAMTS-18 |
| 407) | ADAMTS-19 |
| 408) | ADAMTS-20 |
| 409) | Arg-Gly-Asp |
| 410) | Arg-Gly-Asp-Ser |
| 411) | Arg-Gly-Asp-Ser-Pro-Ala-Ser-Ser-Lys-Pro |
| 412) | Arg-Gly-Glu-Ser |
| 413) | Arg-Phe-Asp-Ser |
| 414) | SPARC |
| 415) | Cys-Asp-Pro-Gly-Tyr-Ile-Gly-Ser-Arg |
| 416) | Cys-Ser-Arg-Ala-Arg-Lys-Gln-Ala-Ala-Ser-Ile-Lys-Val-Ser-Ala- |
| | Asp-Arg |
| 417) | Elastin |
| 418) | Tropelastin |
| 419) | Gly-Arg-Gly-Asp-Ser-Pro-Lys |
| 420) | Gly-Arg-Gly-Asp-Thr-Pro |
| 421) | Laminin |
| 422) | Leu-Gly-Thr-Ile-Pro-Gly |
| 423) | Ser-Asp-Gly-Arg-Gly |
| 424) | Vitronectin |
| 425) | Superfibronectin |
| 426) | Thrombospondin |
| 427) | TIMP-1 |
| 428) | TIMP-2 |
| 429) | TIMP-3 |
| 430) | TIMP-4 |
| 431) | Fibromodulin |
| 432) | Flavoridin |
| 433) | Collagen IV |
| 434) | Collagen V |
| 435) | Collagen VI |
| 436) | Collagen VII |
| 437) | Collagen VIII |
| 438) | Collagen IX |
| 439) | Collagen X |
| 440) | Collagen XI |
| 441) | Collagen XII |
| 442) | Entactin |
| 443) | Fibrillin |
| 444) | Syndecan-1 |
| 445) | Keratan sulfate proteoglycan |

Ambient Oxygen

| | |
|---|---|
| 446) | 0.1-0.5% Oxygen |
| 447) | 0.5-1% Oxygen |
| 448) | 1-2% Oxygen |
| 449) | 2-5% Oxygen |
| 450) | 5-10% Oxygen |
| 451) | 10-20% Oxygen |

Animal Serum

| | |
|---|---|
| 452) | 0.1% Bovine Serum |
| 453) | 0.5% Bovine Serum |
| 454) | 1.0% Bovine Serum |
| 455) | 5.0% Bovine Serum |
| 456) | 10% Bovine Serum |
| 457) | 20% Bovine Serum |
| 458) | 10% Horse Serum |

Interleukins

| | |
|---|---|
| 459) | IL-1 |
| 460) | IL-2 |
| 461) | IL-3 |
| 462) | IL-4 |
| 463) | IL-5 |
| 464) | IL-6 |
| 465) | IL-7 |
| 466) | IL-8 |
| 467) | IL-9 |
| 468) | IL-10 |
| 469) | IL-11 |
| 470) | IL-12 |
| 471) | IL-13 |
| 472) | IL-14 |
| 473) | IL-15 |
| 474) | IL-16 |
| 475) | IL-17 |
| 476) | IL-18 |

Proteases

| | |
|---|---|
| 477) | MMP-1 |
| 478) | MMP-2 |
| 479) | MMP-3 |
| 480) | MMP-4 |
| 481) | MMP-5 |
| 482) | MMP-6 |
| 483) | MMP-7 |
| 484) | MMP-8 |
| 485) | MMP-9 |
| 486) | MMP-10 |
| 487) | MMP-11 |
| 488) | MMP-12 |
| 489) | MMP-13 |
| 490) | MMP-14 |
| 491) | MMP-15 |
| 492) | MMP-16 |
| 493) | MMP-17 |
| 494) | MMP-18 |
| 495) | MMP-19 |
| 496) | MMP-20 |
| 497) | MMP-21 |
| 498) | MMP-22 |
| 499) | MMP-23 |
| 500) | MMP-24 |
| 501) | Cathepsin B |
| 501) | Cathepsin C |
| 503) | Cathepsin D |
| 504) | Cathepsin G |
| 505) | Cathepsin H |
| 506) | Cathepsin L |
| 507) | Trypsin |
| 508) | Pepsin |
| 509) | Elastase |
| 510) | Carboxypeptidase A |
| 511) | Carboxypeptidase B |
| 512) | Carboxypeptidase G |
| 513) | Carboxypeptidase P |
| 514) | Carboxypeptidase W |
| 515) | Carboxypeptidase Y |
| 516) | Chymotrypsin |
| 517) | Plasminogen |
| 518) | Plasmin |
| 519) | u-type Plasminogen activator |
| 520) | t-type Plasminogen activator |
| 521) | Plasminogen activator inhibitor-1 |
| 522) | Carboxypeptidase Z |

Amino Acids

| | |
|---|---|
| 522) | Alanine |
| 523) | Arginine |
| 524) | Asparagine |
| 525) | Aspartic acid |
| 526) | Cysteine |
| 527) | Glutamine |
| 528) | Glutamic acid |
| 529) | Glycine |
| 530) | Histidine |
| 531) | Isoleucine |
| 532) | Leucine |
| 533) | Lysine |
| 534) | Methionine |
| 535) | Phenylalanine |
| 536) | Proline |
| 537) | Serine |
| 538) | Threonine |
| 539) | Tryptophan |
| 540) | Tyrosine |
| 541) | Valine |

Prostaglandins

| | |
|---|---|
| 542) | Prostaglandin A1 |
| 543) | Prostaglandin A2 |
| 544) | Prostaglandin B1 |
| 545) | Prostaglandin B2 |
| 546) | Prostaglandin D2 |
| 547) | Prostaglandin E1 |
| 548) | Prostaglandin E2 |
| 549) | Prostaglandin F1alpha |
| 550) | Prostaglandin F2alpha |
| 551) | Prostaglandin I1 |
| 552) | Prostaglandin I2 |
| 553) | Prostaglandin J2 |
| 554) | 6-Keto-Prostaglandin F1a |
| 555) | 16,16-Dimethyl-Prostaglandin E2 |
| 556) | 15d-Prostaglandin J2 |
| 557) | Prostaglandins pooled (542-556 above) |

Retinoid receptor agonists/Antagonists

| | |
|---|---|
| 558) | Methoprene Acid |
| 559) | All trans retinoic acid |
| 560) | 9-Cis Retinoic Acid |
| 561) | 13-Cis Retinoic Acid |
| 562) | Retinoid agonists pooled (558-561 above) |
| 563) | Retinoid antagonists |
| 564) | Retinoic acid receptor isotype RARalpha |
| 565) | Retinoic acid receptor isotype RARbeta |
| 566) | Retinoic acid receptor isotype RARgamma |
| 567) | Retinoic X receptor isotype RXRalpha |
| 568) | Retinoic X receptor isotype RXRbeta |
| 569) | Retinoic X receptor isotype RARgamma |

Miscellaneous Inducers

| | |
|---|---|
| 570) | Plant lectins |
| 571) | Bacterial lectins |
| 572) | forskolin |
| 573) | Phorbol myristate acetate |
| 574) | Poly-D-lysine |
| 575) | 1,25-dihydroxyvitamin D |
| 576) | Inhibin |
| 577) | Heregulin |
| 578) | Glycogen |
| 579) | Progesterone |
| 580) | IL-1 |
| 581) | Serotonin |
| 582) | Fibronectin - 45 kDa Fragment |
| 583) | Fibronectin - 70 kDa Fragment |
| 584) | glucose |
| 585) | beta mercaptoethanol |
| 586) | heparinase |
| 587) | pituitary extract |
| 588) | chorionic gonadotropin |
| 589) | adrenocorticotropic hormone |
| 590) | thyroxin |
| 591) | Bombesin |
| 592) | Neuromedin B |
| 593) | Gastrin-Releasing Peptide |
| 594) | Epinephrine |
| 595) | Isoproterenol |
| 596) | Ethanol |
| 597) | DHEA |
| 598) | Nicotinic Acid |
| 599) | NADH |
| 600) | Oxytocin |
| 601) | Vasopressin |
| 602) | Vasotocin |
| 603) | Angiotensin I |
| 604) | Angiotensin II |
| 605) | Angiotensin I Converting Enzyme |
| 606) | Angiotensin I Converting Enzyme Inhibitor |
| 607) | Chondroitinase AB |
| 608) | Chondroitinase C |
| 609) | Brain natriuretic peptide |
| 610) | Calcitonin |
| 611) | Calcium ionophore I |
| 612) | Calcium ionophore II |
| 613) | Calcium ionophore III |
| 614) | Calcium ionophore IV |
| 615) | Bradykinin |
| 616) | Albumin |
| 617) | Plasmonate |
| 618) | LIF |
| 619) | PARP inhibitors |
| 620) | Lysophosphatidic acid |
| 621) | (R)-METHANANDAMIDE |
| 622) | 1,25-DIHYDROXYVITAMIN D3 |
| 623) | 1,2-DIDECANOYL-GLYCEROL (10:0) |
| 624) | 1,2-DIOCTANOYL-SN-GLYCEROL |
| 625) | 1,2-DIOLEOYL-GLYCEROL (18:1) |
| 626) | 10-hydroxycamptothecin |
| 627) | 11,12-EPOXYEICOSATRIENOIC ACID |
| 628) | 12(R)-HETE |
| 629) | 12(S)-HETE |
| 630) | 12(S)-HPETE |
| 631) | 12-METHOXYDODECANOIC ACID |
| 632) | 13(S)-HODE |
| 633) | 13(S)-HPODE |
| 634) | 13,14-DIHYDRO-PGE1 |
| 635) | 13-KETOOCTADECADIENOIC ACID |
| 636) | 14,15-EPOXYEICOSATRIENOIC ACID |
| 637) | 1400W |
| 638) | 15(S)-HETE |
| 639) | 15(S)-HPETE |
| 640) | 15-KETOEICOSATETRAENOIC ACID |
| 641) | 17-Allylamino-geldanamycin |
| 642) | 17-OCTADECYNOIC ACID |
| 643) | 17-PHENYL-TRINOR-PGE2 |
| 644) | 1-ACYL-PAF |
| 645) | 1-HEXADECYL-2-ARACHIDONOYL-522) |
| 646) | GLYCEROL |
| 647) | 1-HEXADECYL-2-METHYLGLYCERO-3 PC |
| 648) | 1-HEXADECYL-2-O-ACETYL-GLYCEROL |
| 649) | 1-HEXADECYL-2-O-METHYL-GLYCEROL |
| 650) | 1-OCTADECYL-2-METHYLGLYCERO-3 PC |
| 651) | 1-OLEOYL-2-ACETYL-GLYCEROL |
| 652) | 1-STEAROYL-2-LINOLEOYL-GLYCEROL |
| 653) | 1-STEAROYL-2-ARACHIDONOYL-GLYCEROL |
| 654) | 2,5-ditertbutylhydroquinone |
| 655) | 24(S)-hydroxycholesterol |
| 656) | 24,25-DIHYDROXYVITAMIN D3 |
| 657) | 25-HYDROXYVITAMIN D3 |
| 658) | 2-ARACHIDONOYLGLYCEROL |
| 659) | 2-FLUOROPALMITIC ACID |
| 660) | 2-HYDROXYMYRISTIC ACID |
| 661) | 2-methoxyantimycin A3 |
| 662) | 3,4-dichloroisocoumarin |
| 663) | granzyme B inhibitor |
| 664) | 4-AMINOPYRIDINE |
| 665) | 4-HYDROXYPHENYLRETINAMIDE |
| 666) | 4-OXATETRADECANOIC ACID |
| 667) | 5(S)-HETE |
| 668) | 5(S)-HPETE |
| 669) | 5,6-EPOXYEICOSATRIENOIC ACID |
| 670) | 5,8,11,14-EICOSATETRAYNOIC ACID |
| 671) | 5,8,11-EICOSATRIYNOIC ACID |
| 672) | 5-HYDROXYDECANOATE |
| 673) | 5-iodotubercidin |
| 674) | 5-KETOEICOSATETRAENOIC ACID |
| 675) | 5′-N-Ethylcarboxamidoadenosine (NECA) |
| 676) | 6,7-ADTN HBr |
| 677) | 6-FORMYLINDOLO [3,2-B] CARBAZOLE |
| 678) | 7,7-DIMETHYLEICOSADIENOIC ACID |
| 679) | 8,9-EPOXYEICOSATRIENOIC ACID |
| 680) | 8-methoxymethyl-IBMX |
| 681) | 9(S)-HODE |
| 682) | 9(S)-HPODE |
| 683) | 9,10-OCTADECENOAMIDE |
| 684) | A-3 |
| 685) | AA-861 |
| 686) | acetyl (N)-s-farnesyl-1-cysteine |
| 687) | ACETYL-FARNESYL-CYSTEINE |
| 688) | Ac-Leu-Leu-Nle-CHO |
| 689) | ACONITINE |
| 690) | actinomycin D |
| 691) | ADRENIC ACID (22:4, n-6) |
| 692) | 1 mM |
| 693) | AG-1296 |
| 694) | AG1478 |
| 695) | AG213 (Tyrphostin 47) |
| 696) | AG-370 |
| 697) | AG-490 |
| 698) | AG-879 |
| 699) | AGC |
| 700) | AGGC |
| 701) | Ala-Ala-Phe-CMK |
| 702) | alamethicin |
| 703) | Alrestatin |
| 704) | AM 92016 |
| 704) | AM-251 |
| 706) | AM-580 |
| 707) | AMANTIDINE |
| 708) | AMILORIDE |
| 709) | Amino-1,8-naphthalimide [4-Amino-1,8-522] naphthalimide] |
| 710) | Aminobenzamide (3-ABA) [3-522] aminobenzamide (3-ABA)] |
| 711) | AMIODARONE |
| 712) | ANANDAMIDE (18:2, n-6) |
| 713) | ANANDAMIDE (20:3, n-6) |
| 714) | ANANDAMIDE (20:4, n-6) |
| 715) | ANANDAMIDE (22:4, n-6) |
| 716) | anisomycin |
| 717) | aphidicolin |
| 718) | ARACHIDONAMIDE |
| 719) | ARACHIDONIC ACID (20:4, n-6) |
| 720) | ARACHIDONOYL-PAF |
| 721) | aristolochic acid |
| 722) | Arvanil |
| 723) | ascomycin (FK-520) |
| 724) | B581 |
| 725) | BADGE |
| 726) | bafilomycin A1 |
| 727) | BAPTA-AM |
| 728) | BAY 11-7082 |
| 729) | BAY K-8644 |
| 730) | BENZAMIL |
| 731) | BEPRIDIL |
| 732) | Bestatin |
| 733) | beta-lapachone |
| 734) | Betulinic acid |
| 735) | bezafibrate |
| 736) | Blebbistatin |
| 737) | BML-190 |
| 738) | Boc-GVV-CHO |
| 739) | bongkrekic acid |
| 740) | brefeldin A |
| 741) | Bromo-7-nitroindazole [3-Bromo-7-nitroindazole] |
| 742) | Bromo-cAMP [8-Bromo-cAMP] |
| 743) | Bromo-cGMP [8-Bromo-cGMP] |
| 744) | bumetanide |
| 745) | BW-B 70C |
| 746) | C16 CERAMIDE |
| 747) | C2 CERAMIDE |
| 748) | C2 DIHYDROCERAMIDE |
| 749) | C8 CERAMIDE |
| 750) | C8 CERAMINE |
| 750) | C8 DIHYDROCERAMIDE |
| 751) | CA-074-Me |
| 753) | calpeptin |
| 754) | calphostin C |
| 755) | calyculin A |
| 756) | camptothecin |
| 757) | cantharidin |
| 758) | CAPE |
| 759) | capsacin(E) |
| 760) | capsazepine |
| 761) | CARBACYCLIN |
| 762) | castanospermine |
| 763) | CDC |
| 764) | Cerulenin |
| 765) | CGP-37157 |
| 766) | chelerythrine |
| 767) | CIGLITAZONE |
| 768) | CIMATEROL |
| 769) | CinnGEL 2Me |
| 770) | CIRAZOLINE |
| 771) | CITCO |
| 772) | CLOFIBRATE |
| 773) | clonidine |
| 774) | CLOPROSTENOL Na |
| 775) | clozapine |
| 776) | C-PAF |
| 777) | Curcumin |
| 778) | Cyclo [Arg-Gly-Asp-D-Phe-Val] |
| 779) | cycloheximide |
| 780) | protein synthesis inhibitor |
| 781) | cycloheximide-N-ethylethanoate |
| 782) | cyclopamine |
| 783) | CYCLOPIAZONIC ACID |
| 784) | cyclosporin A |
| 785) | cypermethrin |
| 786) | cytochalasin B |
| 787) | cytochalasin D |
| 788) | D12-PROSTAGLANDIN J2 |
| 789) | D609 |
| 790) | damnacanthal |
| 791) | DANTROLENE |
| 792) | decoyinine |
| 793) | Decylubiquinone |
| 794) | deoxymannojirimycin(1) |
| 795) | deoxynorjrimycin(1) |
| 796) | Deprenyl |
| 797) | DIAZOXIDE |
| 798) | dibutyrylcyclic AMP |
| 799) | dibutyrylcyclic GMP |
| 800) | DICHLOROBENZAMIL |
| 801) | DIHOMO-GAMMA-LINOLENIC ACID |
| 802) | DIHYDROSPHINGOSINE |
| 803) | DIINDOLYLMETHANE |
| 804) | DILTIAZEM |
| 805) | diphenyleneiodonium Cl |
| 806) | dipyridamole |
| 807) | DL-DIHYDROSPHINGOSINE |
| 808) | DL-PDMP |
| 809) | DL-PPMP |
| 810) | DOCOSAHEXAENOIC ACID (22:6 n-3) |
| 811) | DOCOSAPENTAENOIC ACID |
| 812) | DOCOSATRIENOIC ACID (22:3 n-3) |
| 813) | doxorubicin |
| 814) | DRB |
| 815) | E-4031 |
| 816) | E6 berbamine |
| 817) | E-64-d |
| 818) | Ebselen |
| 819) | EHNA HCl |
| 820) | EICOSA-5,8-DIENOIC ACID (20:2 n-12) |
| 821) | EICOSADIENOIC ACID (20:2 n-6) |
| 822) | EICOSAPENTAENOIC ACID (20:5 n-3) |
| 823) | EICOSATRIENOIC ACID (20:3 n-3) |
| 824) | ENANTIO-PAF C16 |
| 825) | epibatidine (+/−) |
| 826) | etoposide |
| 827) | FARNESYLTHIOACETIC ACID |
| 828) | FCCP |
| 829) | FIPRONIL |
| 830) | FK-506 |
| 831) | FLECAINIDE |
| 832) | FLUFENAMIC ACID |
| 833) | FLUNARIZINE |
| 834) | FLUPROSTENOL |
| 835) | FLUSPIRILINE |
| 836) | FPL-64176 |
| 837) | Fumonisin B1 |
| 838) | Furoxan |
| 839) | GAMMA-LINOLENIC ACID (18:3 n-6) |
| 840) | geldanamycin |
| 841) | genistein |
| 842) | GF-109203X |
| 843) | GINGEROL |
| 844) | Gliotoxin |
| 845) | GLIPIZIDE |
| 846) | GLYBURIDE |
| 847) | GM6001 |
| 848) | Go6976 |
| 849) | GRAYANOTOXIN III |
| 850) | GW-5074 |
| 851) | GW-9662 |
| 852) | H7] |
| 853) | H-89 |
| 854) | H9 |
| 855) | HA-1004 |
| 856) | HA1077 |
| 857) | HA14-1 |
| 858) | HBDDE |
| 859) | Helenalin |
| 860) | Hinokitiol |
| 861) | HISTAMINE |
| 862) | HNMPA-(AM)3 |
| 863) | Hoechst 33342 (cell permeable) (BisBenzimide) |
| 864) | Huperzine A [(−)-Huperzine A] |
| 865) | IAA-94 |
| 866) | IB-MECA |
| 867) | IBMX |
| 868) | ICRF-193 |
| 869) | Ikarugamyin |
| 870) | Indirubin |
| 871) | Indirubin-3′-monoxime |
| 872) | indomethacin |
| 873) | juglone |
| 874) | K252A |
| 875) | Kavain (+/−) |
| 876) | KN-62 |
| 877) | KT-5720 |
| 878) | L-744,832 |
| 879) | Latrunculin B |
| 880) | Lavendustin A |
| 881) | L-cis-DILTIAZEM |
| 882) | LEUKOTOXIN A (9,10-EODE) |
| 883) | LEUKOTOXIN B (12,13-EODE) |
| 884) | LEUKOTRIENE B4 |
| 885) | LEUKOTRIENE C4 |
| 886) | LEUKOTRIENE D4 |
| 887) | LEUKOTRIENE E4 |
| 888) | Leupeptin |
| 889) | LFM-A13 |
| 890) | LIDOCAINE |
| 891) | LINOLEAMIDE |
| 892) | LINOLEIC ACID |
| 893) | LINOLENIC ACID (18:3 n-3) |
| 894) | LIPOXIN A4 |
| 895) | L-NAME |
| 896) | L-NASPA |
| 897) | LOPERAMIDE |
| 898) | LY-171883 |
| 899) | LY-294002 |
| 900) | LY-83583 |
| 901) | Lycorine |
| 902) | LYSO-PAF C16 |
| 903) | Manoalide |
| 904) | manumycin A |
| 905) | MAPP, D-erythro |
| 906) | MAPP, L-erythro |
| 907) | mastoparan |
| 908) | MBCQ |
| 909) | MCI-186 |
| 910) | MDL-28170 |
| 911) | MEAD ACID (20:3 n-9) |
| 912) | MEAD ETHANOLAMIDE |
| 913) | methotrexate |
| 914) | METHOXY VERAPAMIL |
| 915) | Mevinolin (lovastatin) |
| 916) | MG-132 |
| 917) | Milrinone |
| 918) | MINOXIDIL |
| 919) | MINOXIDIL SULFATE |
| 920) | MISOPROSTOL, FREE ACID |
| 921) | mitomycin C |
| 922) | ML7 |
| 923) | ML9 |
| 924) | MnTBAP |
| 925) | Monastrol |
| 926) | monensin |
| 927) | MY-5445 |
| 928) | Mycophenolic acid |
| 929) | N,N-DIMETHYLSPHINGOSINE |
| 930) | N9-Isopropylolomoucine |
| 931) | N-ACETYL-LEUKOTRIENE E4 |
| 932) | NapSul-Ile-Trp-CHO |
| 933) | N-ARACHIDONOYLGLYCINE |
| 934) | NICARDIPINE |
| 935) | NIFEDIPINE |
| 936) | NIFLUMIC ACID |
| 937) | Nigericin |
| 938) | NIGULDIPINE |
| 939) | Nimesulide |
| 940) | NIMODIPINE |
| 941) | NITRENDIPINE |
| 942) | N-LINOLEOYLGLYCINE |
| 943) | nocodazole |
| 944) | N-PHENYLANTHRANILIC (CL) |
| 945) | NPPB |
| 946) | NS-1619 |
| 947) | NS-398 |
| 948) | NSC-95397 |
| 949) | OBAA |
| 950) | okadaic acid |
| 951) | oligomycin A |
| 952) | olomoucine |
| 953) | ouabain |
| 954) | PAF C16 |
| 955) | PAF C18 |
| 956) | PAF C18:1 |
| 957) | PALMITYLETHANOLAMIDE |
| 958) | Parthenolide |
| 959) | PAXILLINE |
| 960) | PCA 4248 |
| 961) | PCO-400 |
| 962) | PD 98059 |
| 963) | PENITREM A |
| 964) | pepstatin |
| 965) | PHENAMIL |
| 966) | Phenanthridinone [6(5H)-Phenanthridinone] |
| 967) | Phenoxybenzamine |
| 968) | PHENTOLAMINE |
| 969) | PHENYTOIN |
| 970) | PHOSPHATIDIC ACID, DIPALMITOYL |
| 971) | Piceatannol |
| 972) | pifithrin |
| 973) | PIMOZIDE |
| 974) | PINACIDIL |
| 975) | piroxicam |
| 976) | PP1 |
| 977) | PP2 |
| 978) | prazocin |
| 979) | Pregnenolone 16alpha carbonitrile |
| 980) | PRIMA-1 |
| 981) | PROCAINAMIDE |
| 982) | PROPAFENONE |
| 983) | propidium iodide |
| 984) | propranolol (S-) |
| 985) | puromycin |
| 986) | quercetin |
| 987) | QUINIDINE |
| 988) | QUININE |
| 989) | QX-314 |
| 990) | rapamycin |
| 991) | resveratrol |
| 992) | RETINOIC ACID, ALL TRANS |
| 993) | REV-5901 |
| 994) | RG-14620 |
| 995) | RHC-80267 |
| 996) | RK-682 |
| 997) | Ro 20-1724 |
| 998) | Ro 31-8220 |
| 999) | Rolipram |
| 1000) | roscovitine |
| 1001) | Rottlerin |
| 1002) | RWJ-60475-(AM)3 |
| 1003) | RYANODINE |
| 1004) | SB 202190 |
| 1005) | SB 203580 |
| 1006) | SB-415286 |
| 1007) | SB-431542 |
| 1008) | SDZ-201106 |
| 1009) | S-FARNESYL-L-CYSTEINE ME |
| 1010) | Shikonin |
| 1011) | siguazodan |
| 1012) | SKF-96365 |
| 1013) | SP-600125 |
| 1014) | SPHINGOSINE |
| 1015) | Splitomycin |
| 1016) | SQ22536 |
| 1017) | SQ-29548 |
| 1018) | staurosporine |
| 1019) | SU-4312 |
| 1020) | Suramin |
| 1021) | swainsonine |
| 1022) | tamoxifen |
| 1023) | Tanshinone IIA |
| 1024) | taxol = paclitaxel |
| 1025) | TETRAHYDROCANNABINOL-7-OIC ACID |
| 1026) | TETRANDRINE |
| 1027) | thalidomide |
| 1028) | THAPSIGARGIN |
| 1029) | Thiocitrulline [L-Thiocitrulline HCl] |
| 1030) | Thiorphan |
| 1031) | TMB-8 |
| 1032) | TOLAZAMIDE |
| 1033) | TOLBUTAMIDE |
| 1034) | Tosyl-Phe-CMK (TPCK) |
| 1035) | TPEN |
| 1036) | Trequinsin |
| 1037) | trichostatin-A |
| 1038) | trifluoperazine |
| 1039) | TRIM |
| 1040) | Triptolide |
| 1041) | TTNPB |
| 1042) | Tunicamycin |
| 1043) | tyrphostin 1 |
| 1044) | tyrphostin 9 |
| 1045) | tyrphostin AG-126 |
| 1046) | tyrphostin AG-370 |
| 1047) | tyrphostin AG-825 |
| 1048) | Tyrphostin-8 |
| 1049) | U-0126 |
| 1050) | U-37883A |
| 1051) | U-46619 |
| 1052) | U-50488 |
| 1053) | U73122 |
| 1054) | U-74389G |
| 1055) | U-75302 |
| 1056) | valinomycin |
| 1057) | Valproic acid |
| 1058) | VERAPAMIL |
| 1059) | VERATRIDINE |
| 1060) | vinblastine |
| 1061) | vinpocetine |
| 1062) | W7 |
| 1063) | WIN 55,212-2 |
| 1064) | Wiskostatin |
| 1065) | Wortmannin |
| 1066) | WY-14643 |
| 1067) | Xestospongin C |
| 1068) | Y-27632 |
| 1069) | YC-1 |
| 1070) | Yohimbine |
| 1071) | Zaprinast |
| 1072) | Zardaverine |
| 1073) | ZL3VS |
| 1074) | ZM226600 |
| 1075) | ZM336372 |
| 1076) | Z-prolyl-prolinal |
| 1077) | zVAD-FMK |
| 1078) | Ascorbate |
| 1079) | 5-azacytidine |
| 1080) | 5-azadeoxycytidine |
| 1081) | Hexamethylene bisacetamide (HMBA) |
| 1082) | Sodium butyrate |
| 1083) | Dimethyl sulfoxide. |
| 1084) | Goosecoid |
| 1085) | Glycogen synthase kinase-3 |
| 1086) | Galectin-1 |
| 1087) | Galectin-3 |

Cell Adhesion Molecules

| | |
|---|---|
| 1086) | Cadherin 1 (E-Cadherin) |
| 1087) | Cadherin 2 (N-Cadherin) |
| 1088) | Cadherin 3 (P-Cadherin) |
| 1089) | Cadherin 4 (R-Cadherin) |
| 1090) | Cadherin 5 (VE-Cadherin) |
| 1091) | Cadherin 6 (K-Cadherin) |
| 1092) | Cadherin 7 |
| 1093) | Cadherin 8 |
| 1094) | Cadherin 9 |
| 1095) | Cadherin 10 |
| 1096) | Cadherin 11 (OB-Cadherin) |
| 1097) | Cadherin 12 (BR-Cadherin) |
| 1098) | Cadherin 13 (H-Cadherin) |
| 1099) | Cadherin 14 (same as Cadherin 18) |
| 1100) | Cadherin 15 (M-Cadherin) |
| 1101) | Cadherin 16 (KSP-Cadherin) |
| 1102) | LI Cadherin |

TABLE IV
A set of gene expression markers useful screening for terminal
differentiation in human embryonic progenitor cell lines

| | | | Present(+)/ |
|---|---|---|---|
| | | | Absent(−) |
| Gene | Accession No. | Cell Type | in hEP Cell Lines |
| COL2A1 | NM_001844.3 | Chondrocytes | − |
| COL24A1 | NM_152890.4 | Osteoblasts | low |
| GFAP | NM_002055.2 | Astrocytes | − |
| OLIG2 | NM_005806.1 | Oligodendrocytes | − |
| PLP1 | NM_000533.3 | Oligodendrocytes & | low |
| | | Schwann cells | |
| PRPH | NM_006262.2 | Peripheral Neurons | low |
| ACTA1 | NM_001100.3 | Skeletal Myocytes | − |
| MYF5 | NM_005593.1 | Skeletal Myocytes | − |
| DES | NM_001927.3 | Skeletal Muscle | low-med |
| MYH11 | NM_002474.2 | Smooth Muscle | low-med |
| GCM2 | NM_004752.2 | Parathyroid | − |
| VWF | NM_000552.2 | Endothelial | low |
| PECAM1 | NM_000442.2 | Endothelial | low |
| LY75 | NM_002349.1 | Thymus | − |
| TNMD | NM_022144.1 | Tendon/Ligament | low |
| SCXA | NM_001008271 | Tendon | med |

TABLE V
Exemplary Differentiation Protocols
Adipogenesis Protocol 1

| | |
|---|---|
| Reagents | 1. DMEM (GibcoBRL-Cat# 11965-084) |
| | 2. Calf Serum (GibcoBRL-Cat#16170-078) |
| | 3. Fetal Bovine Serum (GibcoBRL-Cat# 10437-028) |
| | 4. Isobutylmethylxanthine (IBMX; Sigma I-7018) |
| | 5. Dexamethasone (Sigma D-4902) |
| | 6. Insulin (Bovine; Sigma I-5500) |
| | 7. MEM Sodium Pyruvate (100 mM; GibcoBRL Cat#11360-070) |
| | 8. Pen/Strep/Glutamine (100x P/S/G; GibcoBRL Cat#10378-016) |
| Preparation of solutions | 1. 10% Calf Serum/DMEM: 60 mL Calf Serum; 6 mL 100 mM MEM |
| | Sodium Pyruvate; 6 mL 100x P/S/G; 500 mL DMEM. |
| | 2. 10% FBS/DMEM: 60 mL Fetal Bovine Serum (Filter Sterilized); 6 mL |
| | 100 mM MEM Sodium Pyruvate; 6 mL 100x P/S/G; 500 mL DMEM. |
| | 3. IBMX Solution (make fresh): Dissolve IBMX in a solution made of 0.5N |
| | KOH to a final concentration of 0.0115 g/mL; filter sterilize through a |
| | 0.22 mm syringe filter. |
| | 4. Insulin Stock Solution: 167 [symbol] M (1 mg/mL) in 0.02M HCl; Filter |
| | sterilized through 0.22 mm filter; Can store at −20° C. for long term, 4° C. |
| | short term. |
| | 5. Dexamethasone Stock Solutions: Freezer Stock 10 mM of Dex in 100% |
| | ethanol (store at −20° C.); Working Stock: Dilute Freezer stock to 1 mM |
| | in PBS; Filter sterilize and store at 4° C. |
| | 6. MDI Induction Media (10 mL/10 cm plate; 5 mL/6 cm plate); To |
| | required volume of 10% FBS/DMEM add: 1:100 IBMX; 1:1000 |
| | Insulin; 1:1000 Dexamethasone working stock. |
| | 7. Insulin Media (10 mL/10 cm plate; 5 mL/6 cm plate); To required |
| | volume of 10% FBS/DMEM add: 1:100 Insulin (final concentration |
| | 10.0 ug/mL). |
| | 8. Oil red O stock solution (0.5 g/100 ml isopropanol); Just before staining: |
| | mix 60 ml of stock with 40 ml of H₂0, let it sit for 1 hr at RT; filter |
| | through whatman paper 3 MM. |

Procedures

| | |
|---|---|
| Clonal embryonic preadipocyte | Cells are plated in their standard growth media (West et al., 2008, |
| maintenance and passage | Regenerative Medicine vol. 3(3) pp. 287-308; see Supplementary Table I) |
| | and incubated 37° C. in 10% CO₂ and preferable in 5% ambient oxygen. |
| | Cells are frequently observed to prevent them from becoming too |
| | confluent (>70%), until differentiation is induced. |
| Adipocyte Differentiation | 1. Grow embryonic preadipocytes to confluency in their standard growth |
| Protocol | media (West et al., 2008, Regenerative Medicine vol. 3(3) pp. |
| | 287-308). |
| | 2. After two days of post confluency (which is counted as day 0), |
| | stimulate the cells with MDI induction media. |
| | 3. After two days of MDI an induction medium (which is called as day 2) |
| | replace the MDI induction media with Insulin Media and feed |
| | every two days. |
| Staining procedure | 1. Aspirate media, add formaldehyde slowly and let sit for 30 min. |
| | 2. Aspirate formaldehyde and add oil red O solution to cover the well, |
| | leave 1 hr at RT. |
| | 3. Remove the stain and wash with distilled water twice. |
| | Photograph. |

Adipogenesis Protocol 2
Cells are grown to confluence in their standard growth medium (West et al., 2008, Regenerative
Medicine vol. 3(3) pp. 287-308), medium is removed and replaced by serum-free differentiation medium
(DMEM/F12 containing 1 μM bovine insulin, 100 nM hydrocortisone, 10 μg of transferrin/mL, 1 nM
thyronine, 1 μM rosiglitazone, 33 μM biotin, and 17 μM pantothenic acid) to induce adipocyte
differentiation for 3 d. After 3 d of culture, the medium is changed to differentiation medium without
rosiglitazone for another 5 d. The mRNA from cultured cells was extracted at 0, 2, 5, 7 and 14 d of
incubation for transcript analysis as described herein.
Differentiation Factor Protocol 1
Cells are seeded in a 12 well plate precoated with fibronectin (Gibco) at a high density (1.5 × 10⁶
cells/well). Cells are fed three times per week for 14 days with a basal media of knock out DMEM with
penicillin/streptomycin and 16% knock out serum replacement. Individual differentiation factors added to
this basal medium chosen from Table III.
Control five day quiescent cells are plated at 3.0 × 10⁵ cells/well and at confluence fed media with serum
or other growth supplements reduced to 10% of normal values. The cells are refed two days prior to
harvest.
Angiogenesis Protocols

| | |
|---|---|
| Endothelial Formation Protocol | The tube formation assay is carried out on 24-well plates previously |
| (Tube Formation) | coated with 250 [symbol] 1 of matrigel per well (BD Biosciences, cat. # 356237). |
| | The plates are pre-incubated for 30 minutes at 37° C. before seeding the |
| | cells. Subsequently, the cells to be differentiated are seeded at a density |
| | of 5 × 10⁴ cells/well in 1 ml of EGM2 media (LONZA cat. # CC-3162). |
| | The tube formation assays were analyzed at 24 and 96 hours. Cells are |
| | photographed for scoring of the quantity and quality of tube formation |
| | as is well-known in the art. RNA is harvested for Q-PCR and |
| | microarray analysis of gene expression and markers of endothelial cell |
| | differentiation such as the up-regulation of VWF, CDH5 (VE- |
| | Cadherin), CD31, KDR, is assayed. |
| Mural Cell Integration into | Endothelial tubes are generated as described in Endothelial Formation |
| Endothelial Tube Protocol | Protocol (Tube Formation)Above. To measure tube stability and cell |
| | integration, 5 × 10⁴ HUVEC or cells of the present invention including |
| | but not limited to the cell line W10 or cells with markers thereof, are |
| | mixed with 1 × 10⁴ cells that are to be assayed. HUVEC or similar cells |
| | capable of tube formation are labeled with the red dye PKH26 (Sigma, |
| | cat. # MINI26); all other cell lines to be tested for mural cell capacity in |
| | this assay are labeled with the green dye PKH2 (Sigma, cat. # |
| | PKH2GL-1KT). The cell labeling was performed according to the |
| | manufacture's protocol.). The tube formation and mural integration |
| | assays are analyzed at 24 and 96 hours. Fluorescence and transmitted |
| | light images were taken at a magnification of 4x using a Nikon Eclipse |
| | TE 2000-U microscope equipped with an EXFO X-Cite 120 |
| | illumination system. |

Osteogenic Protocol 1
Tissue culture plates are exposed to 12 ug/mL of Type I collagen (gelatin) and 12 ug/mL of vitronectin for
24 hours. This gelatin/vitronectin solution is then aspirated and the cell lines of the present invention are
added at confluent density. Osteogenic media comprising: DMEM (low glucose) with L-Glutamine, 10%
fetal bovine serum, 0.1 uM dexamethasone, 0.2 mM ascorbic acid 2-phosphate, 10 mM glycerol 2-
phosphate, and 100 nM BMP7 is added for 15-21 days. The degree of steogenesis is scored by relative
staining with Alizarin red S performed as follows: Alizarin red S (Sigma) (40 mM) is prepared in dH₂O
and the pH is adjusted to 4.1 using 10% (v/v) ammonium hydroxide. Monolayers in 6-well plates (10 cm2/
well) are washed with PBS and fixed in 10% (v/v) formaldehyde (Sigma-Aldrich) at room
temperature for 15 min. The monolayers are then washed twice with excess dH₂O prior to addition of 1 mL
of 40 mM Alizarin red S (pH 4.1) per well. The plates are incubated at room temperature for 20 min
with gentle shaking. After aspiration of the unincorporated dye, the wells are washed four times with 4 mL
dH₂O while shaking for 5 min. The plates are then left at an angle for 2 min to facilitate removal of
excess water, reaspirated, and then stored at −20° C. prior to dye extraction. Stained monolayers are visualized
by phase microscopy using an inverted microscope (Nikon).
For quantification of staining, 800 uL 10% (v/v) acetic acid is added to each well, and the plate is
incubated at room temperature for 30 min with shaking. The monolayer (loosely attached to the plate) is
scraped from the plate with a cell scraper (Fisher Lifesciences) and transferred with 10% (v/v) acetic acid
to a 1.5-mL microcentrifuge tube with a wide-mouth pipette. After vortexing for 30 s, the slurry is
overlaid with 500 uL mineral oil (Sigma-Aldrich), heated to exactly 85° C. for 10 min, and transferred to
ice for 5 min. Care should be taken at this point to avoid opening of the tubes until fully cooled. The
slurry is then centrifuged at 20,000 g for 15 min and 500 uL of the supernatant is removed to a new 1.5-
mL microcentrifuge tube. 200 uL of 10% (v/v) ammonium hydroxide is added to neutralize the acid. The
pH can be measured at this point to ensure that it is between 4.1 and 4.5. Aliquots (150 uL) of the
supernatant are read in triplicate at 405 nm in 96-well format using opaque-walled, transparent-bottomed
plates (Fisher Lifesciences) as described (Gregory, CA et al, An Alizarin red-based assay of
mineralization by adherent cells in culture: comparison with cetylpyridinium chloride extraction,
Analytical Biochemistry 329 (2004) 77-84).
In vitro conditions to induce chondrogenenesis - Pellet Culture.
Functional differentiation assays utilizing the cells of the present invention can employ micromass and
pellet protocols that are well known in the art as capable of causing bone marrow, adipose, and tooth-
derived mesenchymal stem cells to differentiate into chondrogenic lineages. To demonstrate that
individual cell lines are capable of differentiating into chondrogenic lineages we assayed by qPCR
transcript levels for COL2A1, ACAN, CRTL1, CILP, BGN, and CRTAC1 (CEP-68).
In the case of the Chondrogenic Pellet Protocol:
1. Cells are cultured in gelatin (0.1%) coated Corning tissue culture treated cultureware and
detached with 0.25% trypsin/EDTA (Invitrogen, Carlsbad, CA, Gibco) diluted 1:3 with PBS (Ca, Mg
free). After detachment and addition of growth medium cells are counted using a Coulter counter and
appropriate number of cells needed for experiment (e.g. 10 × 106 or more) are transferred into a sterile
polyproylene tube and spun at 150 g for 5 min at room temperature.
2. The supernatant is aspirated and discarded. The cells are washed with the addition of
Incomplete Chondrogenic Medium consisting of hMSC Chondro BulletKit (PT-3925) to which is added
supplements (Lonza, Basel, Switzerland, Poietics Single-Quots, Cat. # PT-4121). Supplements added to
prepare Incomplete Chondrogenic Medium are: Dexamethasone (PT-4130G), Ascorbate (PT-4131G),
ITS + supplements (4113G), Pyruvate (4114G), Proline (4115G), Gentamicin (4505G), Glutamine (PT-
4140G).
3. Cells are spun at 150 g at room temperature, the supernatant is aspirated and cell the pellet is
resuspended (once more) with 1.0 ml Incomplete Chondrogenic Medium per 7.5 × 10e5 cells, and spun at
150 × g for 5 minutes. The supernatant is aspirated and discarded. The Chondrogenesis culture protocol
as described by Lonza is followed with some modifications (as written below).
4. Cell pellets are resuspended in Complete Chondrogenic medium to a concentration of 5.0 ×
10e5 cells per ml. Complete Chondrogenic Medium consists of Lonza Incomplete Medium plus TGF□3
(Lonza, PT-4124). Sterile lyophilized TGF ®3 is reconstituted with the addition of sterile 4 mM HCl
containing 1 mg/ml BSA to a concentration of 20 ug/ml and is stored after aliquoting at −80° C. Complete
Chondrogenic medium is prepared just before use by the addition of 1 ul of TGF ®3 for each 2 ml of
Incomplete Chondrogenic medium (final TGF ®3 concentration is 10 ng/ml).
5. An aliquot of 0.5 ml (2.5 × 10⁵ cells) of the cell suspension is placed into sterile 15 ml
polypropylene culture tubes. Cells are spun at 150 x g for 5 minutes at room temperature.
6. Following centrifugation the caps of the tubes are loosened one half turn to allow gas
exchange. The tubes are placed in an incubator at 37° C., in a humidified atmosphere of 10% CO₂ and
5% O₂. Pellets are not disturbed for 24 hours.
7. Cell pellets are fed every 2-3 days by completely replacing the medium in each tube by
aspirating the old medium with sterile 1-200 ul pipette tip and adding 0.5 ml of freshly prepared
Complete Chondrogenic Medium to each tube.
8. After replacing the medium and ensuring that the pellet is free-floating, caps are loosened and
tubes returned to the incubator.
9. Pellets are harvested after varying time points in chondrogenic medium and prepared for
histology by fixation with Neutral Buffered Formalin and/or the pellets are combined and prepared for
RNA extraction using Rneasy mini Kits (Qiagen, Germantown, MD, Cat. No. 74104).
The protocol for RNA extraction is followed as described by the Qiagen Handbook. RNA yield
is maximized by using Qiagen's QiaShredder (Cat. # 79654) to homogenize samples following lysis of
cell pellets with RLT buffer (provided in Rneasy mini kits) prior to RNA extraction.
In vitro conditions to induce chondrogenenesis - Micromass Culture.
1. Cells are cultured in gelatin (0.1%) coated Corning tissue culture treated cultureware and
detached with 0.25% trypsin/EDTA (Gibco) diluted 1:3 with PBS (Gibco Ca, Mg free). After detachment
and addition of growth medium cells are counted using a Coulter counter and appropriate number of cells
needed for experiment (e.g. 10 × 10⁶ cells or more) are resuspended at a cell density of 20 × 10⁶ cells/ml in
growth medium.
2. 10 ul aliquots are seeded onto Corning Tissue Culture Treated Polystyrene plates or dishes.
Twenty five or more micromass aliqouts (200,000 cells/10 ul aliquot) are seeded.
3. The seeded micromasses are placed in a humidified incubator at 37° C. with 5% O₂ and 10%
CO₂ for 90 minutes to 2 hours for attachment.
4. Growth medium is added and the following morning is replaced, after aspiration and washing
with PBS (Ca, Mg free), with Complete Chondrogenic Medium (prepared as described above for the
pellet micromasses). For example 6 ml Complete Chondrogenic medium/10 cm dish is added. Cells are
maintainied in a humidified incubator at 37° C. with 5% O₂, 10% CO₂ and chondrogenic medium replaced
with freshly prepared medium every 2-3 days.
5. After varying periods of time in chondrogenic medium RNA is extracted using Qiagen Rneasy
kits (Qiagen Cat. No. 74104) as described in the Qiagen Handbook. RNA yield is maximized by using
Qiagen's QiaShredder (Cat. # 79654 to homogenize samples following lysis of micromasses with RLT
buffer, (which is provided with the Rneasy mini kits) prior to RNA extraction.
An alternative to Lonza Chondrogenic medium is CellGro (Cat. No. 15-013-CV) from Media
Tech. To each 500 ml, the following supplements are added: 5.0 ml Pen/Strep (Gibco Cat. No. 15140),
5.0 ml Glutamax (Gibco Cat. No. 35050), Dexamethasone (Sigma, St. Louis, MO, Cat. No. D1756-100)-
500 ul of 0.1 mM for a final concentration of 0.1 uM; L-Proline (Sigma Cat. No. D49752)-500 ul 0.35M
for a final concentration of 0.35 mM; Ascorbic Acid-2-phosphate (Sigma, Cat. No. 49792, Fluka)-500 ul
0.17M for a final concentration 0.17 mM; ITS Premix (BD, Franklin Lakes, NJ, sterile Cat. No. 47743-
628)-500 ul of 1000x concentrate for a final concentration of 6.25 ug/ml insulin, 6.25 ug/ml transferrin,
6.25 ng/ml selenious acid, serum albumin 1.25 mg/ml, 5.35 ug/ml linoleic acid.
Following addition of constituents above the media is filtered through a 500 ml Corning 0.2
micron filter unit.
As an alternative to Lonza TGF ®3 descibed above we use TGF ®3 (R&D Systems, Minneapolis
MN, Cat. No. 243-B3-010). It is prepared, aliquoted and stored and used similarly to that purchased from
Lonza.
Differentiation in gels containing crosslinked hyaluronic acid and gelatin
The cell lines of the present invention may also be differentiated within hydrogels, including crosslinked
gels containing hyaluronic acid and gelatin with or without added factors listed in Table III. Cells are
trypsinized and suspended at 1-30 × 10e6 cells/ml HyStem-CSS (Glycosan Hydrogel Kit GS319)
according to manufacturer's directions.
1. Preparation of HyStem-CSS:
HyStem (thiol-modified hyaluranan) is dissolved in 1 ml degassed deionized water (taking about 20
minutes). Gelin-S (thiol modified gelatin) is dissolved in 1 ml degassed deionized water and PEGSSDA
(disulfide-containing PEG diacrylate) is dissolved in 0.5 ml degassed deionized water (designated herein
as “PEGSSDA solution”). Then HyStem (1 ml) is mixed with Gelin-S (1 ml) without creating air bubbles,
immediately before use (designated herein as “HyStem:Gelin-S mix”).
2. Retinoic acid and EGF-Containing HyStem-CSS:
In the case of differentiation in HyStem hydrogel containing RA and EGF, 17 million cells are pelleted
and resuspended in 1.4 ml Hystem:Gelin-S mix. Then 0.35 ml of PEGSSDA solution is added, pipetted up
and down, without creating air bubbles, and 100 ul aliquots are quickly placed onto multiple 24 well
inserts (Corning Cat #3413). After gelation, in 20 minutes, encapsulated cells are fed 2 ml growth media
with trans-RA (1 uM) (Sigma, Cat # 2625) or 2 ml growth media with EGF 100 ng/ml (R&D systems Cat#
236-EG). Cells are fed three times weekly. After 28 days, cells are lysed and RNA harvested using
RNeasy micro kits (Qiagen Cat # 74004) for qPCR or microarray analysis as described herein.
3. Differentiation in Hydrogels Containing Crosslinked Hyaluronic Acid and Gelatin to Induce
Chondrogenesis:
Cells are suspended at a density of 20 × 10e6 cells/ml in 1.4 ml Hystem:Gelin-S mix. Then 0.35 ml of
PEGSSDA solution is added, pipetted up and down, without creating air bubbles, and 100 ul aliquots are
quickly placed onto multiple 24 well inserts (Corning Cat #3413). After gelation, in 20 minutes,
encapsulated cells are fed 2 ml Complete Chondrogenic Medium which consists of Lonza Incomplete
Medium plus TGF ®3 (Lonza, PT-4124). Incomplete Chondrogenic Medium consisting of hMSC
Chondro BulletKit (PT-3925) to which is added supplements (Lonza, Basel, Switzerland, Poietics Single-
Quots, Cat. # PT-4121). Supplements added to prepare Incomplete Chondrogenic Medium are:
Dexamethasone (PT-4130G), Ascorbate (PT-4131G), ITS + supplements (4113G), Pyruvate (4114G),
Proline (4115G), Gentamicin (4505G), Glutamine (PT-4140G). Sterile lyophilized TGF ®3 is
reconstituted with the addition of sterile 4 mM HCl containing 1 mg/ml BSA to a concentration of 20 ug/ml
and is stored after aliquoting at −80° C. Complete Chondrogenic medium is prepared just before use by the
addition of 1 ul of TGF ®3 for each 2 ml of Incomplete Chondrogenic medium (final TGF ®3
concentration is 10 ng/ml). Cells are refed three times a week and cultured for a total of 14 days. Cells are
then lysed and RNA harvested using RNeasy micro kits (Qiagen Cat # 74004).
Differentiation of confluent cultures in the presence of EGF
Cell of the present invention are grown to confluence in a 10 cm cell culture dish which may take 0.5-2
weeks depending upon the initial seeding density and the rate of growth of the cell line. Cells are fed
growth media plus 100 ng/ml EGF when they reach confluence and are fed three times a week. After 28
days, cells are lysed and RNA prepared using RNeasy mini kits (Qiagen Cat #71404).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1-8. (canceled)

9. An in vitro endothelial progenitor cell line that expresses the markers PECAM1, vWF, and CDH5 (VE-cadherin).

10. The in vitro endothelial progenitor cell line of claim 1, wherein the endothelial progenitor cell line expresses ITLN1.

11. The in vitro endothelial progenitor cell line of claim 1, wherein the endothelial progenitor cell line expresses ITLN2.

12. The in vitro endothelial progenitor cell line of claim 1, wherein the endothelial progenitor cell line expresses POSTN.

13. The in vitro endothelial progenitor cell line of claim 1, wherein the endothelial progenitor cell line expresses MAMDC2.

14. The in vitro endothelial progenitor cell line of claim 1, wherein the endothelial progenitor cell line expresses APLNR.

15. The in vitro endothelial progenitor cell line of claim 1, wherein the endothelial progenitor cell line expresses APLN.

16. The in vitro endothelial progenitor cell line of claim 1, wherein the endothelial progenitor cell line expresses NPTX2.

17. The in vitro endothelial progenitor cell line of claim 1, wherein the endothelial progenitor cell line is clonal.

18. The in vitro endothelial progenitor cell line of claim 1, wherein the endothelial progenitor cell line is the in vitro progeny of a hES cell.

19. A method of differentiating a hES cell into an endothelial cell comprising:
a) contacting the hES cell with activin and wnt;
b) contacting the cells of a) with activin without wnt;
c) contacting the cells of b) with FGF and BMP;
d) contacting the cells of c) with a TGFβ inhibitor.

19. The method of claim 18 further comprising cloning the endothelial progenitor cell line.

20. The method of claim 19 further comprising expanding the cloned endothelial progenitor cell line.

21. The method of claim 19, wherein the TGFβ inhibitor is SB431542.
